(19) United States
(12) Patent Application Publication
(10) Pub. No.: US 2026/0285980 A1
(43) Pub. Date: Sep. 24, 2026

(54) METHODS FOR TREATING CANCER USING ACTIVATABLE ANTI-CTLA4 ANTIBODY IN COMBINATION WITH PEMBROLIZUMAB

(71) Applicant: ADAGENE PTE. LTD., Singapore (SG)

(72) Inventors: Peter Peizhi LUO, Morgan Hill, CA (US); JiPing ZHA, Suzhou, Jiangsu (CN); Songmao ZHENG, West Harrison, IN (US); Guizhong LIU, Fort Lee, NJ (US); Xiaohong SHE, Suzhou, Jiangsu (CN)

(73) Assignee: ADAGENE PTE. LTD., Singapore (SG)

(21) Appl. No.: 19/474,629

(22) PCT Filed: Apr. 12, 2024
(86) PCT No.: PCT/US2024/024457

§ 371 Date: Oct. 10, 2025

Related U.S. Application Data
(60) Provisional application No. 63/495,968, filed on Apr. 13, 2023.
(60) Provisional application No. 63/584,327, filed on Sep. 21, 2023.
(60) Provisional application No. 63/594,734, filed on Oct. 31, 2023.

(51) Int. Cl. C07K 16/28; A61P 35/00

(52) U.S. Cl. CPC C07K 16/2818; A61P 35/00; C07K 2317/24

(57) ABSTRACT

The present application provides compositions and methods for treating cancers, including advanced-stage metastatic cancer, using an activatable anti-CTLA4 antibody in combination with pembrolizumab.

### CROSS REFERENCE TO RELATED APPLICATIONS

This application claims the priority to U.S. Provisional Application No. 63/495,968 filed Apr. 13, 2023, U.S. Provisional Application No. 63/584,327 filed Sep. 21, 2023, and U.S. Provisional Application No. 63/594,734 filed Oct. 31, 2023, the contents of each of which are incorporated herein by reference in their entireties.

### SUBMISSION OF SEQUENCE LISTING ON ASCII TEXT FILE

The contents of the electronic sequence listing (695402002740SEQLIST.xml; Size: 93,905 bytes; and Date of Creation: Apr. 3, 2024) is herein incorporated by reference in its entirety.

### FIELD OF THE INVENTION

The present application is in the field of cancer therapeutics, and relates to compositions and methods for treating cancers using an antibody that binds to human CTLA4 in combination with the anti-PD-1 antibody pembrolizumab.

### BACKGROUND

CTLA4 is a member of the immunoglobulin (Ig) superfamily of proteins that acts to downregulate T-cell activation and maintain immunogenic homeostasis. It has been shown that in vivo antibody-mediated blockade of CTLA4 enhanced anti-cancer immune responses in a syngeneic murine prostate cancer model (Kwon et al. (1997) Proc Natl Acad Sci USA, 94(15):8099-103). In addition, blockade of CTLA4 function was shown to enhance anti-tumor T cell responses at various stages of tumor growth in tumor-bearing mice (Yang et al. (1997) Cancer Res 57(18):4036-41; Hurwitz et al. (1998) Proc Natl Acad Sci USA 95 (17):10067-7). However, the development of antibody-based therapeutics suitable for human use remains difficult, as translation from pre-clinical animal models to human safety is often poor. Accordingly, a need exists for anti-CTLA4 antibodies that are cross-reactive among different species, such as humans and experimental animals (e.g., mouse, monkey, rat, etc.), to concurrently enable animal model studies and provide suitable human therapeutic candidates. In addition, a need exists for the development of safer anti-CTLA4 antibodies that are only active in certain contexts, such as in the protease-rich tumor microenvironment.

PD-1 is recognized as an important molecule in immune regulation and the maintenance of peripheral tolerance. PD-1 is moderately expressed on naive T, B and NKT cells and up-regulated by T/B cell receptor signaling on lymphocytes, monocytes and myeloid cells (Sharpe, Arlene H et al., The function of programmed cell death 1 and its ligands in regulating autoimmunity and infection. Nature Immunology (2007); 8:239-245).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong, Haidong et al., Tumor-associated B7-H1 promotes T-cell apoptosis: a potential mechanism of immune evasion. Nat Med. 2002 August; 8(8):793-800; Yang, Wanhua et al., PD-1 interaction contributes to the functional suppression of T-cell responses to human uveal melanoma cells in vitro. Invest Ophthalmol Vis Sci. 2008 June; 49(6 (2008): 49: 2518-2525; Ghebeh, Hazem et al., The B7-H1 (PD-L1) T lymphocyte-inhibitory molecule is expressed in breast cancer patients with infiltrating ductal carcinoma: correlation with important high-risk prognostic factors. Neoplasia (2006) 8:190-198; Hamanishi, Junzo et al., Programmed cell death 1 ligand 1 and tumor-infiltrating CD8+ T lymphocytes are prognostic factors of human ovarian cancer. Proc. Natl. Acad. Sci. USA (2007): 104: 3360-3365; Thompson, R Houston, and Eugene D Kwon, Significance of B7-H1 overexpression in kidney cancer. Clinical genitourin Cancer (2006): 5: 206-211; Nomi, Takeo et al., Clinical significance and therapeutic potential of the programmed death-1 ligand/programmed death-1 pathway in human pancreatic cancer. Clinical Cancer Research (2007); 13:2151-2157; Ohigashi, Yuichiro et al., Clinical significance of programmed death-1 ligand-1 and programmed death-1 ligand 2 expression in human esophageal cancer. Clin. Cancer Research (2005): 11: 2947-2953; Inman, Brant A et al., PD-L1 (B7-H1) expression by urothelial carcinoma of the bladder and BCG-induced granulomata: associations with localized stage progression. Cancer (2007): 109: 1499-1505; Shimauchi, Takatoshi et al., Augmented expression of programmed death-1 in both neoplasmatic and nonneoplastic CD4+ T-cells in adult T-cell Leukemia/Lymphoma. Int. J. Cancer (2007): 121:2585-2590; Gao, Qiang et al., Overexpression of PD-L1 significantly associates with tumor aggressiveness and postoperative recurrence in human hepatocellular carcinoma. Clinical Cancer Research (2009) 15:971-979; Nakanishi, Juro et al., Overexpression of B7-H1 (PD-L1) significantly associates with tumor grade and postoperative prognosis in human urothelial cancers. Cancer Immunol Immunother. (2007) 56:1173-1182; Hino et al., Tumor cell expression of programmed cell death-1 is a prognostic factor for malignant melanoma. Cancer (2010): 00: 1-9). Similarly, PD-1 expression on tumor infiltrating lymphocytes was found to mark dysfunctional T cells in breast cancer and melanoma (Ghebeh, Hazem et al., Foxp3+tregs and B7-H1+/PD-1+ T lymphocytes co-infiltrate the tumor tissues of high-risk breast cancer patients: implication for immunotherapy. BMC Cancer. 2008 Feb. 23; 8:57; Ahmadzadeh, Mojgan et al., Tumor antigen-specific CD8 T cells infiltrating the tumor express high levels of PD-1 and are functionally impaired. Blood (2009) 114:1537-1544) and to correlate with poor prognosis in renal cancer (Thompson, R Houston et al., PD-1 is expressed by tumor infiltrating cells and is associated with poor outcome for patients with renal carcinoma. Clinical Cancer Research (2007) 15:1757-1761). Thus, it has been proposed that PD-L1-expressing tumor cells interact with PD-1-expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 have been approved for treating cancer. Pembrolizumab (KEYTRUDA®, Merck Sharp & Dohme LLC, Rahway, NJ, USA) is a potent humanized immunoglobulin G4 (IgG4) mAb with high specificity of binding to the programmed cell death 1 (PD-1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. Keytruda® (pembrolizumab) is indicated for the treatment of patients across a number of indications and is indicated for the first-line treatment of patients with unresectable or metastatic CRC that is microsatellite instability-high or mismatch repair deficient (MSI-H/dMMR). Pembrolizumab is the current standard of care for first line MSI-H/dMMR mCRC.

### BRIEF SUMMARY

The present application provides methods of treating cancer with an activatable anti-CTLA4 antibody of the disclosure in combination with the anti-PD-1 antibody pembrolizumab. The present application further provides methods for treating cancer with an anti-CTLA4 antibody, pembrolizumab and at least one additional therapeutic agent.

In one aspect, provided herein is a method of treating a cancer in a subject (e.g., a human patient), comprising administering to the subject (e.g., a human patient): (a) an effective amount of an activatable antibody, wherein the activatable antibody comprises: a polypeptide comprising, from N-terminus to C-terminus, a masking moiety (MM), a cleavable moiety (CM), and an anti-CTLA4 antibody as described herein, wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221); and (b) an effective amount of pembrolizumab. The MM and CM, from N-terminus to C-terminus comprises an amino acid sequence EVGSYPNPSSDCVPYYYACAY SGRSAGGGGTPLGLAGSGGS (SEQ ID NO: 200). In particular embodiments, the MM and the CM are covalently attached to the N-terminus of the light chain of the anti-CTLA4 antibody. In some embodiments, the MM and CM, from N-terminus to C-terminus comprises an amino acid sequence that has at least 90% or at least 95% sequence identity to SEQ ID NO:200.

In some embodiments, the activatable anti-CTLA4 antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75).

In some such embodiments, the activatable anti-CTLA4 antibody, upon cleavage of the CM, comprises: a) a heavy chain variable region comprising the amino acid sequence of SEQ ID NO: 87, and a light chain variable region comprising the amino acid sequence of SEQ ID NO: 100. In some embodiments, the activatable anti-CTLA4 antibody comprises a heavy chain variable region comprising an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 87, and/or a light chain variable region comprising the amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 100.

In some embodiments, the activatable anti-CTLA4 antibody comprises a heavy chain comprising the amino acid sequence of SEQ ID NO:320 and a light chain comprising the amino acid sequence of SEQ ID NO:322. The activatable antibody having heavy chain SEQ ID NO: 320 and light chain SEQ ID NO: 322 is referred to as TY22404. In some embodiments, the activatable antibody comprises a heavy chain comprising the amino acid sequence of SEQ ID NO: 320 and a light chain comprising the amino acid sequence of SEQ ID NO:322. In some embodiments, the activatable anti-CTLA4 antibody comprises a heavy chain having at least 90% (e.g., at least 92%, at least 93%, at least 94%, at least 95%, at least 96%, at least 97%, at least 98% or at least 99%) sequence identity to the amino acid sequence of SEQ ID NO:320. In some embodiments, the activatable anti-CTLA4 antibody comprises a light chain having at least 90% (e.g., at least 92%, at least 93%, at least 94%, at least 95%, at least 96%, at least 97%, at least 98% or at least 99%) sequence identity to the amino acid sequence of SEQ ID NO:321. In some embodiments, the activatable anti-CTLA4 antibody comprises a heavy chain having at least 90% (e.g., at least 92%, at least 93%, at least 94%, at least 95%, at least 96%, at least 97%, at least 98% or at least 99%) sequence identity to the amino acid sequence of SEQ ID NO:320. In some embodiments, the activatable anti-CTLA4 antibody comprises a light chain having at least 90% (e.g., at least 92%, at least 93%, at least 94%, at least 95%, at least 96%, at least 97%, at least 98% or at least 99%) sequence identity to the amino acid sequence of SEQ ID NO:322. In some embodiments, the activatable antibody is TY22404.

In any of the foregoing embodiments, the activatable anti-CTLA4 antibody (e.g., TY22404), when administered in combination with pembrolizumab, can be administered at a dose that provides a steady-state plasma concentration of the cleaved antibody (i.e., active antibody following cleavage of the masking moiety (MM) and cleavable moiety (CM)) of from about 50 nM to about 100 nM. In some embodiments, the activatable anti-CTLA4 antibody is administered at a dose that provides a steady-state plasma concentration of the cleaved antibody of from about 50 nM to about 100 nM. In some embodiments, the activatable anti-CTLA4 antibody is administered at a dose that provides a steady-state plasma concentration of the cleaved antibody of from about 50 nM to about 75 nM. In some embodiments, the activatable anti-CTLA4 antibody is administered at a dose that provides a steady-state plasma concentration of the cleaved antibody of from about 70 nM to about 80 nM. In some embodiments, the activatable anti-CTLA4 antibody is administered at a dose that provides a steady-state plasma concentration of the cleaved antibody of from about 100 nM to about 175 nM. In some embodiments, the activatable anti-CTLA4 antibody is administered at a dose that provides a steady-state plasma concentration of the cleaved antibody of from about 100 nM to about 200 nM. In some embodiments, the activatable anti-CTLA4 antibody is administered at a dose that provides a steady-state plasma concentration of the cleaved antibody of from about 125 nM to about 200 nM. In some embodiments, the activatable anti-CTLA4 antibody is administered at a dose that provides a steady-state plasma concentration of the cleaved antibody of from about 100 nM to about 150 nM. In some embodiments, the activatable anti-CTLA4 antibody is administered at a dose that provides a steady-state plasma concentration of the cleaved antibody of from about 125 nM to about 175 nM. In some embodiments, the activatable anti-CTLA4 antibody is administered at a dose that provides a steady-state plasma concentration of the cleaved antibody of from about 125 nM to about 150 nM. In some embodiments, the activatable anti-CTLA4 antibody is administered at a dose that provides a steady-state plasma concentration of the cleaved antibody of from about 150 nM to about 200 nM. In some embodiments, the activatable anti-CTLA4 antibody is administered at a dose that provides a steady-state plasma concentration of the cleaved antibody of from about 200 nM to about 600 nM. In some embodiments, the activatable anti-CTLA4 antibody is administered at a dose that provides a steady-state plasma concentration of the cleaved antibody of from about 200 nM to about 400 nM. In some embodiments, the activatable anti-CTLA4 antibody is administered at a dose that provides a steady-state plasma concentration of the cleaved antibody of from about 300 nM to about 500 nM. In some embodiments, the activatable anti-CTLA4 antibody is administered at a dose that provides a steady-state plasma concentration of the cleaved antibody of from about 400 nM to about 600 nM. In some of the foregoing embodiments, the plasma concentration can be measured at the trough level of the activatable anti-CTLA4 antibody (i.e., minimal concentration of each dosing cycle). For instance, the plasma concentration of a particular cycle can be measured immediately prior to administering a dose at the next cycle.

In some embodiments, the activatable anti-CTLA4 antibody and pembrolizumab is administered in combination with one or more additional therapeutic agents. In some such embodiments, the activatable anti-CTLA4 antibody is administered at a dose that provides a steady-state plasma concentration of the cleaved antibody of from about 50 nM to about 150 nM. In other such embodiments, the activatable anti-CTLA4 antibody is administered at a dose that provides a steady-state plasma concentration of the cleaved antibody of from about 50 nM to about 100 nM. In other such embodiments, the activatable anti-CTLA4 antibody is administered at a dose that provides a steady-state plasma concentration of the cleaved antibody of from about 50 nM to about 75 nM. In other such embodiments, the activatable anti-CTLA4 antibody is administered at a dose that provides a steady-state plasma concentration of the cleaved antibody of from about 75 nM to about 100 nM.

In any of the foregoing embodiments, the activatable anti-CTLA4 antibody (e.g., TY22404), when administered in combination with pembrolizumab, can be administered at a dose that provides a plasma concentration ratio of cleaved antibody to uncleaved antibody at steady-state of from about 0.3 to about 1.0 at the trough level of a particular dosing cycle. In some embodiments, the activatable anti-CTLA4 antibody can be administered at a dose that provides a plasma concentration ratio of cleaved antibody to uncleaved antibody at steady-state of from about 0.3 to about 0.8 at the trough level of a particular dosing cycle. In some embodiments, the activatable anti-CTLA4 antibody can be administered at a dose that provides a plasma concentration ratio of cleaved antibody to uncleaved antibody at steady-state of from about 0.5 to about 0.8 at the trough level of a particular dosing cycle. In some embodiments, the activatable anti-CTLA4 antibody can be administered at a dose that provides a plasma concentration ratio of cleaved antibody to uncleaved antibody at steady-state of from about 0.7 to about 1.0 at the trough level of a particular dosing cycle.

In one aspect, the disclosure provides a method of treating a cancer in a subject (e.g., a human patient), comprising administering to the subject (e.g., a human patient) an effective amount of an activatable anti-CTLA4 antibody described above (e.g., TY22404) in combination with pembrolizumab, wherein the activatable anti-CTLA4 antibody is administered at a dose of from about 3 mg/kg to about 30 mg/kg. In some embodiments, the activatable anti-CTLA4 antibody (e.g., TY22404) is administered at a dose of from about 10 mg/kg to about 20 mg/kg. In some embodiments, the activatable anti-CTLA4 antibody (e.g., TY22404) is administered at a dose of from about 20 mg/kg to about 30 mg/kg. In some embodiments, the activatable anti-CTLA4 antibody (e.g., TY22404) is administered at a dose of about 3 mg/kg. In some embodiments, the activatable anti-CTLA4 antibody (e.g., TY22404) is administered at a dose of about 5 mg/kg. In some embodiments, the activatable anti-CTLA4 antibody (e.g., TY22404) is administered at a dose of about 6 mg/kg. In some embodiments, the activatable anti-CTLA4 antibody (e.g., TY22404) is administered at a dose of about 8 mg/kg. In some embodiments, the activatable anti-CTLA4 antibody (e.g., TY22404) is administered at a dose of about 10 mg/kg. In some embodiments, the activatable anti-CTLA4 antibody (e.g., TY22404) is administered at a dose of about 8 mg/kg. In some embodiments, the activatable anti-CTLA4 antibody (e.g., TY22404) is administered at a dose of about 20 mg/kg. In some embodiments, the activatable anti-CTLA4 antibody (e.g., TY22404) is administered at a dose of about 25 mg/kg. In some embodiments, the activatable anti-CTLA4 antibody (e.g., TY22404) is administered at a dose of about 30 mg/kg. In any of the foregoing embodiments, the activatable anti-CTLA4 antibody can be administered once every three weeks or once every six weeks. For instance, in some embodiments, the activatable anti-CTLA4 antibody can be administered at a dose of 10 mg/kg once every three weeks or once every six weeks. In other embodiments, the activatable anti-CTLA4 antibody can be administered at a dose of 20 mg/kg once every three weeks or once every six weeks. In other embodiments, the activatable anti-CTLA4 antibody can be administered at a dose of 30 mg/kg once every three weeks or once every six weeks.

In some embodiments, the activatable anti-CTLA4 antibody is administered at a first higher dose (e.g., between about 10 mg/kg and about 100 mg/kg) for at least one treatment cycle (as defined herein) followed by a lower dose (e.g., between about 3 mg/kg to about 20 mg/kg) in subsequent cycles. The higher initial doses will also be referred to herein as loading doses while the remining doses will also be referred to herein as maintenance doses. As set forth in the examples, administration of at least one loading dose results in establishing steady-state plasma concentrations of the activatable anti-CTLA4 antibody more rapidly. In some embodiments, the loading doses of the activatable anti-CTLA4 antibody are administered in combination with pembrolizumab. In other embodiments, the loading doses of the activatable anti-CTLA4 antibody are not administered in combination with pembrolizumab. In such embodiments, the pembrolizumab is administered in combination with the maintenance doses of the activatable anti-CTLA4 antibody, as set forth herein.

In one embodiment, the activatable anti-CTLA4 antibody is administered at a dose of from about 20 mg/kg to a dose of about 50 mg/kg for at least one treatment cycle (e.g., one to three treatment cycles) and at a dose of from about 6 mg/kg to 20 mg/kg in subsequent treatment cycles (e.g., once every three weeks or once every six weeks). In one embodiment, the activatable anti-CTLA4 antibody is administered at a dose of from about 20 mg/kg to a dose of about 50 mg/kg for at least one treatment cycle (e.g., one to three treatment cycles) and at a dose of from about 10 mg/kg to 20 mg/kg in subsequent treatment cycles (e.g., once every three weeks or once every six weeks). In one embodiment, the activatable anti-CTLA4 antibody is administered at a dose of from about 20 mg/kg to a dose of about 50 mg/kg for at least one treatment cycle (e.g., one to three treatment cycles) and at a dose of from about 6 mg/kg to about 10 mg/kg in subsequent treatment cycles (e.g., once every three weeks or once every six weeks). In one embodiment, the activatable anti-CTLA4 antibody is administered at a dose of from about 20 mg/kg to a dose of about 50 mg/kg for at least one treatment cycle (e.g., one to three treatment cycles) and at a dose of about 6 mg/kg in subsequent treatment cycles In one embodiment, the activatable anti-CTLA4 antibody is administered at a dose of about 20 mg/kg to a dose of about 50 mg/kg for at least one treatment cycle (e.g., one to three treatment cycles) and at a dose of about 10 mg/kg in subsequent treatment cycles (e.g., once every three weeks or once every six weeks).

In one embodiment, the activatable anti-CTLA4 antibody is administered at a dose of from about 20 mg/kg to a dose of about 40 mg/kg for at least one treatment cycle (e.g., one to three treatment cycles) and at a dose of from about 6 mg/kg to 20 mg/kg in subsequent treatment cycles (e.g., once every three weeks or once every six weeks). In one embodiment, the activatable anti-CTLA4 antibody is administered at a dose of from about 20 mg/kg to a dose of about 40 mg/kg for at least one treatment cycle (e.g., one to three treatment cycles) and at a dose of from about 10 mg/kg to 20 mg/kg in subsequent treatment cycles (e.g., once every three weeks or once every six weeks). In one embodiment, the activatable anti-CTLA4 antibody is administered at a dose of from about 20 mg/kg to a dose of about 40 mg/kg for at least one treatment cycle (e.g., one to three treatment cycles) and at a dose of from about 6 mg/kg to about 10 mg/kg in subsequent treatment cycles (e.g., once every three weeks or once every six weeks). In one embodiment, the activatable anti-CTLA4 antibody is administered at a dose of from about 20 mg/kg to a dose of about 40 mg/kg for at least one treatment cycle (e.g., one to three treatment cycles) and at a dose of about 6 mg/kg in subsequent treatment cycles In one embodiment, the activatable anti-CTLA4 antibody is administered at a dose of about 20 mg/kg to a dose of about 40 mg/kg for at least one treatment cycle (e.g., one to three treatment cycles) and at a dose of about 10 mg/kg in subsequent treatment cycles (e.g., once every three weeks or once every six weeks).

In one embodiment, the activatable anti-CTLA4 antibody is administered at a dose of about 20 mg/kg for at least one treatment cycle (e.g., one to three treatment cycles) and at a dose of about 6 mg/kg in subsequent treatment cycles (e.g., once every three weeks). In one embodiment, the activatable anti-CTLA4 antibody is administered at a dose of about 20 mg/kg for one treatment cycle and at a dose of about 6 mg/kg in subsequent treatment cycles (e.g., once every three weeks). In another embodiment, the activatable anti-CTLA4 antibody is administered at a dose of about 20 mg/kg for two treatment cycles and at a dose of about 6 mg/kg in subsequent treatment cycles (e.g., once every three weeks). In one embodiment, the activatable anti-CTLA4 antibody is administered at a single dose of 20 mg/kg (loading dose) followed by a dose of 6 mg/kg (maintenance dose) three weeks later. The maintenance dose (6 mg/kg) is then administered once every three weeks in subsequent cycles. In another embodiment, the activatable anti-CTLA4 antibody is administered twice at a dose of 20 mg/kg (loading dose) once every three weeks followed by a dose of 6 mg/kg (maintenance dose) three weeks later. The maintenance dose (6 mg/kg) is then administered once every three weeks in subsequent cycles.

In one embodiment, the activatable anti-CTLA4 antibody is administered at a dose of about 20 mg/kg for at least one treatment cycle (e.g., one to three treatment cycles) and at a dose of about 10 mg/kg in subsequent treatment cycles (e.g., once every three weeks). In one embodiment, the activatable anti-CTLA4 antibody is administered at a dose of about 20 mg/kg for one treatment cycle and at a dose of about 10 mg/kg in subsequent treatment cycles (e.g., once every three weeks). In another embodiment, the activatable anti-CTLA4 antibody is administered at a dose of about 20 mg/kg for two treatment cycles and at a dose of about 10 mg/kg in subsequent treatment cycles (e.g., once every three weeks). In one embodiment, the activatable anti-CTLA4 antibody is administered at a single dose of 20 mg/kg (loading dose) followed by a dose of 10 mg/kg (maintenance dose) three weeks later. The maintenance dose (10 mg/kg) is then administered once every three weeks in subsequent cycles. In another embodiment, the activatable anti-CTLA4 antibody is administered twice at a dose of 20 mg/kg (loading dose) once every three weeks followed by a dose of 10 mg/kg (maintenance dose) three weeks later. The maintenance dose (10 mg/kg) is then administered once every three weeks in subsequent cycles.

In one embodiment, the activatable anti-CTLA4 antibody is administered at a dose of from about 30 mg/kg to about 50 mg/kg (e.g., 30 mg/kg, 35 mg/kg, 40 mg/kg, 45 mg/kg or 50 mg/kg) for at least one treatment cycle (e.g., one to three treatment cycles) and at a dose of about 10 mg/kg in subsequent treatment cycles (e.g., once every three weeks). In one embodiment, the activatable anti-CTLA4 antibody is administered at a dose of from about 30 mg/kg to about 50 mg/kg (e.g., 30 mg/kg, 35 mg/kg, 40 mg/kg, 45 mg/kg or 50 mg/kg) for one treatment cycle and at a dose of about 10 mg/kg in subsequent treatment cycles (e.g., once every three weeks). In another embodiment, the activatable anti-CTLA4 antibody is administered at a dose of from about 30 mg/kg to about 50 mg/kg (e.g., 30 mg/kg, 35 mg/kg, 40 mg/kg, 45 mg/kg or 50 mg/kg) for two treatment cycles and at a dose of about 10 mg/kg in subsequent treatment cycles (e.g., once every three weeks). In one embodiment, the activatable anti-CTLA4 antibody is administered at a single dose of from about 30 mg/kg to about 50 mg/kg (e.g., 30 mg/kg, 35 mg/kg, 40 mg/kg, 45 mg/kg or 50 mg/kg) (loading dose) followed by a dose of 10 mg/kg (maintenance dose) three weeks later. The maintenance dose (10 mg/kg) is then administered once every three weeks in subsequent cycles. In another embodiment, the activatable anti-CTLA4 antibody is administered twice at a dose of from about 30 mg/kg to about 50 mg/kg (e.g., 30 mg/kg, 35 mg/kg, 40 mg/kg, 45 mg/kg or 50 mg/kg) (loading dose) once every three weeks followed by a dose of 10 mg/kg (maintenance dose) three weeks later. The maintenance dose (10 mg/kg) is then administered once every three weeks in subsequent cycles.

In some embodiments, pembrolizumab is administered at a dose of from about 100 mg to about 300 mg once every three weeks. In some such embodiments, the activatable anti-CTLA4 antibody (e.g., TY22404) and pembrolizumab are both administered once every three weeks.

In some embodiments, the activatable anti-CTLA4 antibody (e.g., TY22404) is administered at a dose of from about 6 mg/kg to about 10 mg/kg once every three to six weeks and pembrolizumab is administered at a dose of from about 200 mg to about 600 mg once every six weeks. In some such embodiments, the activatable anti-CTLA4 antibody (e.g., TY22404) and pembrolizumab are both administered once every six weeks.

In some embodiments, the activatable anti-CTLA4 antibody (e.g., TY22404) is administered at a dose of 6 mg/kg once every three to six weeks and pembrolizumab is administered at a dose of from about 200 mg to about 600 mg once every six weeks. In some such embodiments, the activatable anti-CTLA4 antibody (e.g., TY22404) and pembrolizumab are both administered once every six weeks. In some such embodiments, the aforementioned dosing regimen is a maintenance dose and may be preceded by administration of one or more loading doses of the activatable anti-CTLA4 antibody, as set forth herein.

In some embodiments, the activatable anti-CTLA4 antibody (e.g., TY22404) is administered at a dose of 10 mg/kg once every three to six weeks and pembrolizumab is administered at a dose of from about 200 mg to about 600 mg once every six weeks. In some such embodiments, the activatable anti-CTLA4 antibody (e.g., TY22404) and pembrolizumab are both administered once every six weeks. In some such embodiments, the aforementioned dosing regimen is a maintenance dose and may be preceded by administration of one or more loading doses of the activatable anti-CTLA4 antibody, as set forth herein.

In some embodiments, the activatable anti-CTLA4 antibody (e.g., TY22404) is administered at a dose of 20 mg/kg once every three to six weeks and pembrolizumab is administered at a dose of from about 200 mg to about 600 mg once every six weeks. In some such embodiments, the activatable anti-CTLA4 antibody (e.g., TY22404) and pembrolizumab are both administered once every six weeks. In some such embodiments, the aforementioned dosing regimen is a maintenance dose and may be preceded by administration of one or more loading doses of the activatable anti-CTLA4 antibody, as set forth herein.

In some embodiments, the activatable anti-CTLA4 antibody (e.g., TY22404) is administered at a dose of 30 mg/kg once every three to six weeks and pembrolizumab is administered at a dose of from about 200 mg to about 600 mg once every six weeks. In some such embodiments, the activatable anti-CTLA4 antibody (e.g., TY22404) and pembrolizumab are both administered once every six weeks. In some such embodiments, the aforementioned dosing regimen is a maintenance dose and may be preceded by administration of one or more loading doses of the activatable anti-CTLA4 antibody, as set forth herein.

In some embodiments, the activatable anti-CTLA4 antibody (e.g., TY22404) is administered at a dose of from about 6 mg/kg to about 10 mg/kg once every three to six weeks and pembrolizumab is administered at a dose of from about 100 mg to about 300 mg once every three weeks.

In one embodiment, the activatable anti-CTLA4 antibody (e.g., TY22404) is administered to the subject (e.g., a human patient) at dose of from about 1 mg/kg to about 30 mg/kg, from about 6 mg/kg to about 10 mg/kg from about 10 mg/kg to about 20 mg/kg, or from about 10 mg/kg to about 20 mg/kg. In some such embodiments, the activatable anti-CTLA4 antibody (e.g., TY22404) is administered to the subject (e.g., a human patient) at a dose of about 6 mg/kg. In some embodiments, the activatable anti-CTLA4 antibody (e.g., TY22404) is administered to the subject (e.g., a human patient) at a dose of about 10 mg/kg (e.g., 10 mg/kg). In some such embodiments, the activatable anti-CTLA4 antibody (e.g., TY22404) is administered to the subject (e.g., a human patient) once every three weeks. In other such embodiments, the activatable anti-CTLA4 antibody (e.g., TY22404) is administered to the subject (e.g., a human patient) once every six weeks. In some embodiments, the activatable anti-CTLA4 antibody (e.g., TY22404) is administered to the subject (e.g., a human patient) at a dose of about 20 mg/kg (e.g., 20 mg/kg). In some such embodiments, the activatable anti-CTLA4 antibody (e.g., TY22404) is administered to the subject (e.g., a human patient) once every three weeks. In other such embodiments, the activatable anti-CTLA4 antibody (e.g., TY22404) is administered to the subject (e.g., a human patient) once every six weeks. In some embodiments, the activatable anti-CTLA4 antibody (e.g., TY22404) is administered to the subject (e.g., a human patient) at a dose of about 25 mg/kg (e.g., 25 mg/kg). In some such embodiments, the activatable anti-CTLA4 antibody (e.g., TY22404) is administered to the subject (e.g., a human patient) once every three weeks. In other such embodiments, the activatable anti-CTLA4 antibody (e.g., TY22404) is administered to the subject (e.g., a human patient) once every six weeks. In some embodiments, the activatable anti-CTLA4 antibody (e.g., TY22404) is administered to the subject (e.g., a human patient) at a dose of about 30 mg/kg (e.g., 30 mg/kg). In some such embodiments, the activatable anti-CTLA4 antibody (e.g., TY22404) is administered to the subject (e.g., a human patient) once every three weeks. In other such embodiments, the activatable anti-CTLA4 antibody (e.g., TY22404) is administered to the subject (e.g., a human patient) once every six weeks. In any of the foregoing embodiments, pembrolizumab can be administered in combination with the activatable anti-CTLA4 antibody on the same day of a particular dosing regimen or on different days of a particular regimen. In some embodiments, both the activatable anti-CTLA4 antibody and pembrolizumab are administered on the first day of a three week or six week dosing regimen. In some such embodiments, the aforementioned dosing regimen is a maintenance dose and may be preceded by administration of one or more loading doses of the activatable anti-CTLA4 antibody, as set forth herein.

In some embodiments, the pembrolizumab is administered at a dose of from about 100 mg to about 300 mg once every three weeks. In some embodiments, the pembrolizumab is administered at a dose of about 200 mg once every three weeks. In some such embodiments, the activatable anti-CTLA4 antibody is administered concurrently with the pembrolizumab. For instance, the activatable anti-CTLA4 antibody and the pembrolizumab can each be administered to a subject (e.g., a human patient) in need thereof on day 1 of a three week or a six week dosing schedule.

In some embodiments, the activatable anti-CTLA4 antibody (e.g., TY22404) and pembrolizumab are administered to a subject (e.g., a human patient) in need thereof, wherein the activatable anti-CTLA4 antibody (e.g., TY22404) is administered at a dose of 6 mg/kg once every three weeks and the pembrolizumab is administered at a dose of 200 mg once every three weeks. In some embodiments, the activatable anti-CTLA4 antibody and pembrolizumab are administered concurrently. In some such embodiments, the aforementioned dosing regimen is a maintenance dose and may be preceded by administration of one or more loading doses of the activatable anti-CTLA4 antibody, as set forth herein.

In some embodiments, the activatable anti-CTLA4 antibody (e.g., TY22404) and pembrolizumab are administered to a subject (e.g., a human patient) in need thereof, wherein the activatable anti-CTLA4 antibody (e.g., TY22404) is administered at a dose of 10 mg/kg once every three weeks and the pembrolizumab is administered at a dose of 200 mg once every three weeks. In some embodiments, the activatable anti-CTLA4 antibody and pembrolizumab are administered concurrently. In some such embodiments, the aforementioned dosing regimen is a maintenance dose and may be preceded by administration of one or more loading doses of the activatable anti-CTLA4 antibody, as set forth herein.

In some embodiments, the activatable anti-CTLA4 antibody (e.g., TY22404) and pembrolizumab are administered to a subject (e.g., a human patient) in need thereof, wherein the activatable anti-CTLA4 antibody (e.g., TY22404) is administered at a dose of 20 mg/kg once every three weeks and the pembrolizumab is administered at a dose of 200 mg once every three weeks. In some embodiments, the activatable anti-CTLA4 antibody and pembrolizumab are administered concurrently. In some such embodiments, the aforementioned dosing regimen is a maintenance dose and may be preceded by administration of one or more loading doses of the activatable anti-CTLA4 antibody, as set forth herein.

In some embodiments, the activatable anti-CTLA4 antibody (e.g., TY22404) and pembrolizumab are administered to a subject (e.g., a human patient) in need thereof once every six weeks. In some such embodiments, the activatable anti-CTLA4 antibody is administered at a dose of from about 3 mg/kg to about 20 mg/kg (e.g., 3 mg/kg, 6 mg/kg, 10 mg/kg, 15 mg/kg, 20 mg/kg, 25 mg/kg or 30 mg/kg) and the pembrolizumab is administered at a dose of from about 200 mg to about 400 mg (e.g., about 400 mg). In particular embodiments, the activatable anti-CTLA4 antibody and pembrolizumab are administered concurrently. In some such embodiments, the aforementioned dosing regimen is a maintenance dose and may be preceded by administration of one or more loading doses of the activatable anti-CTLA4 antibody, as set forth herein.

In some embodiments, the activatable anti-CTLA4 antibody (e.g., TY22404) and pembrolizumab are administered to a subject (e.g., a human patient) in need thereof, wherein the activatable anti-CTLA4 antibody (e.g., TY22404) is administered at a dose of 6 mg/kg once every six weeks and the pembrolizumab is administered at a dose of 200 mg once every six weeks. In some embodiments, the activatable anti-CTLA4 antibody and pembrolizumab are administered concurrently. In some such embodiments, the aforementioned dosing regimen is a maintenance dose and may be preceded by administration of one or more loading doses of the activatable anti-CTLA4 antibody, as set forth herein.

In some embodiments, the activatable anti-CTLA4 antibody (e.g., TY22404) and pembrolizumab are administered to a subject (e.g., a human patient) in need thereof, wherein the activatable anti-CTLA4 antibody (e.g., TY22404) is administered at a dose of 10 mg/kg once every six weeks and the pembrolizumab is administered at a dose of 200 mg once every six weeks. In some embodiments, the activatable anti-CTLA4 antibody and pembrolizumab are administered concurrently. In some such embodiments, the aforementioned dosing regimen is a maintenance dose and may be preceded by administration of one or more loading doses of the activatable anti-CTLA4 antibody, as set forth herein.

In some embodiments, the activatable anti-CTLA4 antibody (e.g., TY22404) and pembrolizumab are administered to a subject (e.g., a human patient) in need thereof, wherein the activatable anti-CTLA4 antibody (e.g., TY22404) is administered at a dose of 20 mg/kg once every six weeks and the pembrolizumab is administered at a dose of 200 mg once every six weeks. In some embodiments, the activatable anti-CTLA4 antibody and pembrolizumab are administered concurrently. In some such embodiments, the aforementioned dosing regimen is a maintenance dose and may be preceded by administration of one or more loading doses of the activatable anti-CTLA4 antibody, as set forth herein.

In some embodiments, the activatable anti-CTLA4 antibody (e.g., TY22404) and pembrolizumab are administered to a subject (e.g., a human patient) in need thereof, wherein the activatable anti-CTLA4 antibody (e.g., TY22404) is administered at a dose of 30 mg/kg once every six weeks and the pembrolizumab is administered at a dose of 200 mg once every six weeks. In some embodiments, the activatable anti-CTLA4 antibody and pembrolizumab are administered concurrently. In some such embodiments, the aforementioned dosing regimen is a maintenance dose and may be preceded by administration of one or more loading doses of the activatable anti-CTLA4 antibody, as set forth herein.

In some embodiments according to any one of the methods described above, the cancer is resistant or refractory to a prior therapy, wherein the prior therapy is an inhibitor of CTLA4, PD-1, or a PD-1 ligand. In some embodiments, the subject (e.g., a human patient) is resistant to or has relapsed from a prior therapy, wherein the prior therapy is an inhibitor of CTLA4, PD-1, or a PD-1 ligand. In some embodiments, the prior therapy is an inhibitor of CTLA4, such as ipilimumab. In some embodiments, the prior therapy is an inhibitor of PD-1, such as an anti-PD-1 antibody. In some embodiments, the prior therapy is an inhibitor of a PD-1 ligand (e.g., PD-L1), for example an anti-PD-L1 antibody.

In some embodiments according to any one of the methods described above, the cancer is liver cancer, a cancer of the digestive system (e.g., colon cancer, colorectal cancer), lung cancer, bone cancer, heart cancer, brain cancer, kidney cancer, bladder cancer, a hematological cancer (e.g., leukemia), skin cancer, breast cancer, thyroid cancer, pancreatic cancer, a head and/or neck cancer, an eye-related cancer, a male reproductive system cancer (e.g., prostate cancer, testicular cancer), or a female reproductive system cancer (e.g., uterine cancer, cervical cancer). In some embodiments, the cancer is a solid cancer. In some embodiments, the cancer is pancreatic cancer. In some embodiments, the cancer is advanced-stage cancer. In some embodiments, the cancer is colorectal cancer. In some embodiments, the cancer is cervical cancer. In some embodiments, the cancer is neuroendocrine cancer. In some embodiments, the cancer is endometrium cancer. In some embodiments, the cancer is cecum cancer. In some embodiments, the cancer is ovarian cancer. In one embodiment, cancer includes but is not limited to colorectal cancer, gastric cancer, gastroesophageal junction cancer, esophageal cancer, endometrial cancer, or head and neck cancer. In another embodiment, cancer includes but is not limited to melanoma, non-small cell lung cancer (NSCLC), small cell lung cancer (SCLC), head and neck squamous cell cancer (HNSCC), classical Hodgkin lymphoma (cHL), primary mediastinal large B-cell lymphoma (PMBCL), urothelial carcinoma, microsatellite instability-high or mismatch repair deficient cancer, microsatellite instability-high or mismatch repair deficient colorectal cancer, gastric cancer, esophageal cancer, cervical cancer, hepatocellular carcinoma (HCC), Merkel cell carcinoma (MCC), renal cell carcinoma (RCC), endometrial carcinoma, tumor mutational burden-high (TMB-H) cancer, cutaneous squamous cell carcinoma (cSCC), or triple negative breast cancer (TNBC). The present disclosure is related to a method of treating cancer in a subject (e.g., a human patient) in need thereof, comprising administering to the subject (e.g., a human patient) a combination therapy comprising at least two pharmaceutical compositions.

In some embodiments, the activatable anti-CTLA4 antibody and pembrolizumab are both administered intravenously. In some embodiments, the activatable anti-CTLA4 antibody is administered subcutaneously. In some embodiments, the activatable anti-CTLA4 antibody and pembrolizumab are administered intravenously or subcutaneously once every three weeks. In some embodiments, the activatable anti-CTLA4 antibody and pembrolizumab are administered intravenously or subcutaneously once every six weeks. In some embodiments, the subject (e.g., a human patient) receives at least 4 cycles of treatment with the activatable anti-CTLA4 antibody and pembrolizumab. In some embodiments, the subject (e.g., a human patient) further receives a further maintenance treatment (e.g., after four or more cycles) comprising administering to the subject (e.g., a human patient) an effective amount of the activatable anti-CTLA4 antibody about once every four weeks to about once every twelve weeks (e.g., once every 4, 6, 8, 10, or 12 weeks). In some embodiments, the doses of the activatable anti-CTLA4 antibody and pembrolizumab may be administered at the same time. In other embodiments, the doses of the activatable anti-CTLA4 antibody and pembrolizumab may be administered at different times. For instance, pembrolizumab may be administered from about 0.5 hours to about 5 hours prior to or following administration of the activatable anti-CTLA4 antibody on day 1 of the dosing schedule (e.g., three week dosing schedule).

In some embodiments according to any one of the methods described above, the subject is human.

It is to be understood that one, some, or all of the properties of the various embodiments described above and herein may be combined to form other embodiments of the present application. These and other aspects of the present application will become apparent to one of skill in the art. These and other embodiments of the present application are further described by the detailed description that follows.

### BRIEF DESCRIPTION OF FIGURES

FIGS. 1A-1B show response to activatable CTLA4-antibody TY22404 in combination with pembrolizumab. FIG. 1A shows swimmer plot for patients treated with TY22404+pembrolizumab. As of this data-cut, 4 patients were still receiving treatment. All patients had at least 2 cycles of treatments. The longest treatment period is more than 8 cycles.er plot. FIG. 1B shows Waterfall plot for patients with target lesion measurements available for both baseline and post-baseline (n=10) who were treated with TY22404+pembrolizumab during dose escalation. One patient (10 mg/kg, Q3W; PD due to new lesion) did not have complete post-treatment target lesion measurement and is not.

FIG. 2 shows serum IFN-γ levels upon treatment by TY22404 monotherapy or in combination with pembrolizumab. Serum IFN-γ were quantified using Mesoscale Discovery (MSD) Technologies' V-Plex Proinflammatory Panel 1.

FIGS. 3A and 3B show representative minimal physiologically-based pharmacokinetic (mPBPK) model fitting to observed TY22404 pharmacokinetic (PK) data (e.g., plasma intact and cleaved) at a dose of 10 mg/kg once three weeks. FIG. 3A shows concentration of intact (uncleaved) antibody over three dosing schedules. FIG. 3B shows concentration of cleaved antibody over three dosing schedules.

FIG. 4 shows circulation of TY22404 following administration to various species.

FIG. 5 shows representative PK simulation (mean with 95% CI) for TY22404 using mPBPK modeling at 10 mg/kg Q3W.

FIG. 6 shows that at steady-state (SS), maximum cleaved TY22404 tumor interstitial fluid (ISF) concentration at 10 mg/kg Q3W dosing is predicted on average to be significantly higher than ipilimumab for 3 mg/kg Q3W*4 doses or 1 mg/kg Q6W, respectively in the tumor microenvironment (TME).

FIG. 7 shows that at steady-state (SS), cleaved TY22404 plasma or serum concentration at 10 mg/kg Q3W dosing is predicted to have reduced active drug exposures in normal tissue compared with ipilimumab at 3 mg/kg Q3W*4 or 1 mg/kg Q6W.

FIG. 8 shows predicted drug concentrations of cleaved TY22404 at various dosing cycles.

FIG. 9 shows PK modeling to predict the effect of a single loading dose followed by maintenance doses of TY22404. The model predicts that steady-state plasma concentrations can be achieved after one dosing cycle.

FIG. 10 shows mechanism-based safety modeling for TY22404 and ipilimumab in combination with anti-PD1 antibodies. The vertical lines represent model-estimated PD marker levels from TY22404 at the specified dosing regimen on day 42 after dosing. TY22404 shows far less treatment related adverse effects relative to ipilimumab when the antibodies are administered in combination with various anti-PD1 antibodies.

FIG. 11 shows the ability of TY22404 to overcome Pembrolizumab resistance in a 3L cervical patient.

FIG. 12 shows greater fluctuations in mPBPK model-predicted tumor cleaved PK, as well as reduced cleaved AUC/Cmax compared with 10 mg/kg Q3W dosing of TY22404.

FIG. 13 shows mPBPK model predicted tumor cleaved PK at steady-state is above the upper bound in vitro EC₉₀ (approximately 90 nM) for cleaved TY22404 (human T cell binding).

FIG. 14 shows that mPBPK model can characterize plasma and tumor PK in tumor-bearing mice well after a 10 mg/kg single dose, allowing the estimation of tumor cleavage parameter of TY22404.

FIG. 15 shows mPBKB model-predicted mean plasma cleaved TY22404 concentration (nM) over time for different dosing regimens with alternative loading doses as compared to the target concentration for clinical effect based on population exposure-response (E-R) analysis.

FIGS. 16A and 16B show mPBPK model-predicted mean cleaved TY22404 concentration over time for different dosing regimens with alternative loading doses in tumor interstitial fluid (ISF; FIG. 16A) or leaky normal tissue (FIG. 16B).

### DETAILED DESCRIPTION

### I. Definitions

Unless otherwise defined herein, scientific, and technical terms used in connection with the present application shall have the meanings that are commonly understood by those of ordinary skill in the art. Further, unless otherwise required by context, singular terms shall include pluralities and plural terms shall include the singular. Generally, nomenclatures used in connection with, and techniques of, antibody engineering, immunotherapy, cell and tissue culture, molecular biology, immunology, microbiology, genetics and protein and nucleic acid chemistry described herein are those well-known and commonly used in the art.

The term “antibody” is used herein in the broadest sense and specifically covers monoclonal antibodies (including full length monoclonal antibodies), polyclonal antibodies, multispecific antibodies (e.g., bispecific antibodies, trispecific antibodies), and antibody fragments (e.g., Fab, Fab′, Fab′-SH, F(ab′)₂, Fv and/or a single-chain variable fragment or scFv) so long as they exhibit the desired biological activity.

An “antibody fragment” or “antigen-binding fragment” refers to a molecule other than an intact antibody that comprises a portion of an intact antibody that binds the antigen to which the intact antibody binds. The antibody fragment retains the ability to bind specifically to the antigen bound by the full-length antibody, e.g. fragments that retain one or more CDR regions, e.g. all six CDRs. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments.

In some embodiments, the term “antibody” refers to an antigen-binding protein (i.e., immunoglobulin) having a basic four-polypeptide chain structure consisting of two identical heavy (H) chains and two identical light (L) chains. Each L chain is linked to an H chain by one covalent disulfide bond, while the two H chains are linked to each other by one or more disulfide bonds depending on the H chain isotype. Each heavy chain has, at the N-terminus, a variable region (abbreviated herein as V_{H}) followed by a constant region. The heavy chain constant region is comprised of three domains, C_{H1}, C_{H2} and C_{H3}. Each light chain has, at the N-terminus, a variable region (abbreviated herein as V_{I}) followed by a constant region at its other end. The light chain constant region is comprised of one domain, C_{L}. The V_{L} is aligned with the V_{H} and the C_{L} is aligned with the first constant domain of the heavy chain (CH1). The pairing of a V_{H} and V_{L} together forms a single antigen-binding site. An IgM antibody consists of 5 of the basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain.

The V_{H} and V_{L} regions can be further subdivided into regions of hypervariability, termed hyper-variable regions (HVR) based on structural and sequence analysis. HVRs are interspersed with regions that are more conserved, termed framework regions (FW) (see e.g., Chen et al. (1999) J. Mol. Biol. (1999) 293, 865-881). Each V_{H} and V_{L} is composed of three HVRs and four FWs, arranged from amino-terminus to carboxy-terminus in the following order: FW-1_HVR-1_FW-2_HVR-2_FW-3_HVR-3_FW4. Throughout the present application, the three HVRs of the heavy chain are referred to as HVR-H1, HVR-H2, and HVR-H3. Similarly, the three HVRs of the light chain are referred to as HVR-L1, HVR-L2, and HVR-L3.

As used herein, the term “CDR” or “CDRs” means complementarity determining region(s) in an immunoglobulin variable region or the non-contiguous antigen combining sites found within the variable region of both heavy and light chain polypeptides. As used herein, CDRs are defined using the Kabat numbering system, unless otherwise indicated. See e.g., Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273:927-948 (1997); MacCallum et al., J. Mol. Biol. 262:732-745 (1996); Abhinandan and Martin, Mol. Immunol., 45:3832-3839 (2008); Lefranc M. P. et al., Dev. Comp. Immunol., 27:55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. CDR prediction algorithms and interfaces are known in the art, including, for example, Abhinandan and Martin, Mol. Immunol., 45:3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein.

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. Within light and heavy chains, the variable and constant regions are joined by a “J” region of about 12 or more amino acids, with the heavy chain also including a “D” region of about 10 or more amino acids (see e.g., Fundamental Immunology Ch. 7 (Paul, W., ed., 2^{nd} ed. Raven Press, N.Y). (1989)).

The L chain from any vertebrate species can be assigned to one of two clearly distinct types, called kappa and lambda, based on the amino acid sequences of their constant domains. Depending on the amino acid sequence of the constant domain of their heavy chains (CH), antibodies can be assigned to different classes or isotypes. There are five classes of antibodies: IgA, IgD, IgE, IgG, and IgM, having heavy chains designated α (alpha), δ (delta), ε (epsilon), γ (gamma), and μ (mu), respectively. The IgG class of antibody can be further classified into four subclasses IgG1, IgG2, IgG3, and IgG4 by the gamma heavy chains, Y1-Y4, respectively.

The term “CTLA4” is used in the present application, and includes the human CTLA4 (e.g., UniProt accession number P16410), as well as variants, isoforms, and species homologs thereof (e.g., mouse CTLA4 (UniProt accession number P09793), rat CTLA4 (UniProt accession number Q9Z1A7), dog CTLA4 (UniProt accession number Q9XSI1), cynomolgus monkey CTLA4 (UniProt accession number G7PL88), etc.). Accordingly, an anti-CTLA4 antibody, as defined and disclosed herein, may also bind CTLA4 from species other than human. In other cases, an anti-CTLA4 antibody may be completely specific for the human CTLA4 and may not exhibit species or other types of cross-reactivity.

The term “CTLA4 antibody” refers to an antibody, as defined herein, capable of binding to human CTLA4.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

“PD-1 antagonist” means any chemical compound or biological molecule that blocks binding of PD-L1 expressed on a cancer cell to PD-1 expressed on an immune cell (T cell, B cell or Natural Killer T cell) and in specific embodiments also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCDIL1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and in specific embodiments blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

“Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table B. Pembrolizumab has been approved by the U.S. FDA as described in the Prescribing Information for KEYTRUDA® (Merck Sharp & Dohme LLC, Rahway, NJ, USA, initial U.S. approval 2014, updated March 2021).

As used herein, a “pembrolizumab variant” or “a variant thereof” pertaining to a pembrolizumab sequence means a monoclonal antibody that comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

The term “epitope” refers to a part of an antigen to which an antibody (or antigen-binding fragment thereof) binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope can include various numbers of amino acids in a unique spatial conformation. Methods of determining spatial conformation of epitopes include, for example, x-ray crystallography, 2-dimensional nuclear magnetic resonance, deuterium and hydrogen exchange in combination with mass spectrometry, or site-directed mutagenesis, or all methods used in combination with computational modeling of antigen and its complex structure with its binding antibody and its variants (see e.g., Epitope Mapping Protocols in Methods in Molecular Biology, Vol. 66, G. E. Morris, Ed. (1996)). Once a desired epitope of an antigen is determined, antibodies to that epitope can be generated, e.g., using the techniques described herein. The generation and characterization of antibodies may also elucidate information about desirable epitopes. From this information, it is then possible to competitively screen antibodies for binding to the same epitope. An approach to achieve this is to conduct cross-competition studies to find antibodies that competitively bind with one another, i.e., the antibodies compete for binding to the antigen. A high throughput process for “binning” antibodies based upon their cross-competition is described in PCT Publication No. WO 03/48731.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see e.g., Flatman et al., J. Chromatogr. B 848:79-87 (2007).

As used herein, “sequence identity” between two polypeptide sequences indicates the percentage of amino acids that are identical between the sequences. The amino acid sequence identity of polypeptides can be determined conventionally using known computer programs such as Bestfit, FASTA, or BLAST (see e.g., Pearson, Methods Enzymol. 183:63-98 (1990); Pearson, Methods Mol. Biol. 132:185-219 (2000); Altschul et al., J. Mol. Biol. 215:403-410 (1990); Altschul et al., Nucelic Acids Res. 25:3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

As used herein, the term “binds”, “binds to”, “specifically binds” “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence

The term “treat”, “treating”, or “treatment”, with reference to a certain disease condition in a mammal, refers causing a desirable or beneficial effect in the mammal having the disease condition. The desirable or beneficial effect may include reduced frequency or severity of one or more symptoms of the disease (i.e., tumor growth and/or metastasis, or other effect mediated by the numbers and/or activity of immune cells, and the like), or arrest or inhibition of further development of the disease, condition, or disorder. In the context of treating cancer in a mammal, the desirable or beneficial effect may include inhibition of further growth or spread of cancer cells, death of cancer cells, inhibition of reoccurrence of cancer, reduction of pain associated with the cancer, or improved survival of the mammal. The effect can be either subjective or objective. For example, if the mammal is human, the human may note improved vigor or vitality or decreased pain as subjective symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement, such as sonograms, nuclear magnetic resonance testing and positron emissions testing.

The term “prevent” or “preventing,” with reference to a certain disease condition in a mammal, refers to preventing or delaying the onset of the disease, or preventing the manifestation of clinical or subclinical symptoms thereof.

As used herein, a “subject”, “patient”, or “individual” may refer to a human or a non-human animal. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

An “effective amount” refers to at least an amount effective, at dosages and for periods of time necessary, to achieve one or more desired or indicated effects, including a therapeutic or prophylactic result. An effective amount can be provided in one or more administrations. For purposes of the present application, an effective amount of antibody, drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition (e.g., an effective amount as administered as a monotherapy or combination therapy). Thus, an “effective amount” may be considered in the context of administering one or more therapeutic agents, and a single agent may be considered to be given in an effective amount if, in conjunction with one or more other agents, a desirable result may be or is achieved.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

As used herein, a “baseline level” or “baseline value” refers to a level or a value of a subject before the subject (e.g., a human patient) begins a treatment, such as an anti-CTLA4 antibody treatment.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A patient who “does not have an effective response” to treatment refers to a patient who does not have any one of extending survival (including overall survival and progression free survival); resulting in an objective response (including a complete response or a partial response); or improving signs or symptoms of cancer.

The methods and techniques of the present application are generally performed according to methods well known in the art and as described in various general and more specific references that are cited and discussed throughout the present specification unless otherwise indicated. Such references include, e.g., Sambrook and Russell, Molecular Cloning, A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990). Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)).

As used herein, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present application described herein include “comprising,” “consisting,” and “consisting essentially of” aspects and embodiments.

The term “about X-Y” used herein has the same meaning as “about X to about Y.”

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

### II. Methods of Treatment

The present application provides methods for treating cancers in a subject (e.g., a human patient) using an activatable anti-CTLA4 antibody of the disclosure. Any one of the anti-CTLA4 antibodies (including full-length antibodies and antigen-binding fragments thereof) in Section III “Anti-CTLA4 Antibodies” may be used in the methods described herein.

In some embodiments, there is provided a method of treating a cancer in a subject (e.g., a human patient), wherein the cancer is resistant or refractory to an inhibitor of CTLA-4, PD-1 or a PD-1 ligand (e.g., PD-L1 or PD-L2), comprising administering to the subject (e.g., a human patient) an effective amount of an activatable anti-CTLA4 antibody in combination with pembrolizumab, wherein the antibody comprises: (a) a heavy chain variable region comprising an HVR-H1 comprising the amino acid sequence of SEQ ID NO: 23, an HVR-H2 comprising the amino acid sequence of SEQ ID NO: 35, and an HVR-H3 comprising the amino acid sequence of SEQ ID NO: 45, and/or a light chain variable region comprising an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some embodiments, the cancer is resistant or refractory to an anti-PD-1 antibody. In some embodiments, the cancer is resistant or refractory to a different anti-CTLA4 antibody, such as ipilimumab. In some embodiments, the cancer is resistant or refractory to an anti-PD-L1 antibody. In some embodiments, the cancer is a solid cancer, such as advanced-stage and/or metastatic cancer. In some embodiments, the antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100. In some embodiments, the antibody comprises a human IgG1 Fc region, such as a wildtype IgG1 Fc region or a variant that has enhanced ADCC activity. I

In some embodiments, there is provided a method of treating a cancer in a subject (e.g., a human patient), comprising administering to the subject (e.g., a human patient) an effective amount of an anti-CTLA4 antibody as disclosed herein in combination with pembrolizumab, wherein the activatable anti-CTLA4 antibody is administered at a dose of from about 3 mg/kg to about 20 mg/kg. In some embodiments, the activatable anti-CTLA4 is administered at a dose of about 3 mg/kg. In some embodiments, the activatable anti-CTLA4 antibody is administered at a dose of about 5 mg/kg. In some embodiments, the activatable anti-CTLA4 antibody is administered at a dose of about 6 mg/kg. In some embodiments, the activatable anti-CTLA4 antibody is administered at a dose of about 8 mg/kg. In some embodiments, the activatable anti-CTLA4 antibody is administered at a dose of about 10 mg/kg. In some embodiments, the activatable anti-CTLA4 antibody is administered at a dose of about 20 mg/kg. In some embodiments, the activatable anti-CTLA4 antibody is administered at a dose of about 25 mg/kg. In some embodiments, the activatable anti-CTLA4 antibody is administered at a dose of about 30 mg/kg. In some of the foregoing embodiments, the activatable anti-CTLA4 antibody and the pembrolizumab are administered once every three weeks. In some of the foregoing embodiments, the activatable anti-CTLA4 antibody and the pembrolizumab are administered once every six weeks. In some of the foregoing embodiments, the activatable anti-CTLA4 antibody and the pembrolizumab are administered for at least 5 (e.g., 6, 7, 8, 9, 10, 11, 12, 13, 14, 15, 16, 17, 18, 19 or 20) treatment cycles.

In one aspect, the disclosure provides a method of treating a cancer in a subject (e.g., a human patient), comprising administering to the subject (e.g., a human patient) an effective amount of an activatable anti-CTLA4 antibody described above (e.g., TY22404) in combination with pembrolizumab, wherein the activatable anti-CTLA4 antibody is administered at a dose of from about 3 mg/kg to about 30 mg/kg. In some embodiments, the activatable anti-CTLA4 antibody (e.g., TY22404) is administered at a dose of from about 5 mg/kg to about 10 mg/kg. In some embodiments, the activatable anti-CTLA4 antibody (e.g., TY22404) is administered at a dose of from about 10 mg/kg to about 20 mg/kg. In some embodiments, the activatable anti-CTLA4 antibody (e.g., TY22404) is administered at a dose of from about 20 mg/kg to about 30 mg/kg. In some embodiments, the activatable anti-CTLA4 antibody (e.g., TY22404) is administered at a dose of about 3 mg/kg. In some embodiments, the activatable anti-CTLA4 antibody (e.g., TY22404) is administered at a dose of about 5 mg/kg. In some embodiments, the activatable anti-CTLA4 antibody (e.g., TY22404) is administered at a dose of about 6 mg/kg. In some embodiments, the activatable anti-CTLA4 antibody (e.g., TY22404) is administered at a dose of about 8 mg/kg. In some embodiments, the activatable anti-CTLA4 antibody (e.g., TY22404) is administered at a dose of about 10 mg/kg. In some embodiments, the activatable anti-CTLA4 antibody (e.g., TY22404) is administered at a dose of about 8 mg/kg. In some embodiments, the activatable anti-CTLA4 antibody (e.g., TY22404) is administered at a dose of about 20 mg/kg. In some embodiments, the activatable anti-CTLA4 antibody (e.g., TY22404) is administered at a dose of about 25 mg/kg. In some embodiments, the activatable anti-CTLA4 antibody (e.g., TY22404) is administered at a dose of about 30 mg/kg. In any of the foregoing embodiments, the activatable anti-CTLA4 antibody can be administered once every three weeks or once every six weeks. For instance, in some embodiments, the activatable anti-CTLA4 antibody can be administered at a dose of 10 mg/kg once every three weeks or once every six weeks. In other embodiments, the activatable anti-CTLA4 antibody can be administered at a dose of 20 mg/kg once every three weeks or once every six weeks. In other embodiments, the activatable anti-CTLA4 antibody can be administered at a dose of 30 mg/kg once every three weeks or once every six weeks.

In some embodiments, the activatable anti-CTLA4 antibody is administered at a first higher dose (e.g., between about 10 mg/kg and about 100 mg/kg) for at least one treatment cycle (as defined herein) followed by a lower dose (e.g., between about 3 mg/kg to about 20 mg/kg) in subsequent cycles. The higher initial doses will also be referred to herein as loading doses while the remining doses will also be referred to herein as maintenance doses. In some embodiments, the loading doses of the activatable anti-CTLA4 antibody are administered in combination with pembrolizumab. In other embodiments, the loading doses of the activatable anti-CTLA4 antibody are not administered in combination with pembrolizumab. In such embodiments, the pembrolizumab is administered in combination with the maintenance doses of the activatable anti-CTLA4 antibody, as set forth herein.

In one embodiment, the activatable anti-CTLA4 antibody is administered at a dose of about 20 mg/kg for at least one treatment cycle (e.g., one to three treatment cycles) and at a dose of about 5-10 mg/kg or 6-8 mg/kg in subsequent treatment cycles (e.g., once every three weeks). In one embodiment, the activatable anti-CTLA4 antibody is administered at a dose of about 20 mg/kg for at least one treatment cycle (e.g., one to three treatment cycles) and at a dose of about 6 mg/kg in subsequent treatment cycles (e.g., once every three weeks). In one embodiment, the activatable anti-CTLA4 antibody is administered at a dose of about 20 mg/kg for at least one treatment cycle (e.g., one to three treatment cycles) and at a dose of about 10 mg/kg in subsequent treatment cycles (e.g., once every three weeks).

In one embodiment, the activatable anti-CTLA4 antibody is administered at a dose of about 10 mg/kg for one treatment cycle and at a dose of about 6 mg/kg in subsequent treatment cycles (e.g., once every three weeks or once every six weeks). In one embodiment, the activatable anti-CTLA4 antibody is administered at a dose of about 10 mg/kg for two treatment cycle and at a dose of about 6 mg/kg in subsequent treatment cycles (e.g., once every three weeks or once every six weeks).

In one embodiment, the activatable anti-CTLA4 antibody is administered at a dose of about 20 mg/kg for one treatment cycle and at a dose of about 6 mg/kg in subsequent treatment cycles (e.g., once every three weeks or once every six weeks). In one embodiment, the activatable anti-CTLA4 antibody is administered at a dose of about 20 mg/kg for one treatment cycle and at a dose of about 10 mg/kg in subsequent treatment cycles (e.g., once every three weeks or once every six weeks).

In another embodiment, the activatable anti-CTLA4 antibody is administered at a dose of about 20 mg/kg for two treatment cycles and at a dose of about 6 mg/kg in subsequent treatment cycles (e.g., once every three weeks or once every six weeks). In another embodiment, the activatable anti-CTLA4 antibody is administered at a dose of about 20 mg/kg for two treatment cycles and at a dose of about 10 mg/kg in subsequent treatment cycles (e.g., once every three weeks or once every six weeks).

In one embodiment, the activatable anti-CTLA4 antibody is administered at a single dose of 20 mg/kg (loading dose) followed by a dose of 6 mg/kg (maintenance dose) three weeks later. The maintenance dose (e.g., 6 mg/kg) is then administered once every three weeks in subsequent cycles. In another embodiment, the activatable anti-CTLA4 antibody is administered twice at a dose of 20 mg/kg (loading dose) once every three weeks followed by a dose of 6 mg/kg (maintenance dose) three weeks later. The maintenance dose (6 mg/kg) is then administered once every three weeks in subsequent cycles.

In one embodiment, the activatable anti-CTLA4 antibody is administered at a single dose of 20 mg/kg (loading dose) followed by a dose of 10 mg/kg (maintenance dose) three weeks later. The maintenance dose (e.g., 10 mg/kg) is then administered once every three weeks in subsequent cycles. In another embodiment, the activatable anti-CTLA4 antibody is administered twice at a dose of 20 mg/kg (loading dose) once every three weeks followed by a dose of 10 mg/kg (maintenance dose) three weeks later. The maintenance dose (10 mg/kg) is then administered once every three weeks in subsequent cycles.

In some embodiments, the activatable anti-CTLA4 antibody is administered at a dose of about 20 mg/kg for one treatment cycle and at a dose of from about 5 mg/kg to about 10 mg/kg in subsequent treatment cycles (e.g., once every three weeks or once every six weeks). For instance, in one embodiment, the activatable anti-CTLA4 antibody is administered at an initial (loading) dose of 20 mg/kg and then is administered additional doses (maintenance doses) of from about 5 mg/kg to about 10 mg/kg every three weeks. In some embodiments, the activatable anti-CTLA4 antibody is administered at a dose of about 20 mg/kg for two treatment cycles and at a dose of from about 5 mg/kg to about 10 mg/kg in subsequence treatment cycles (e.g., once every three weeks). In one embodiment, the activatable anti-CTLA4 antibody is administered at an initial (loading) dose of 20 mg/kg, administered another loading dose of 20 mg/kg three weeks later, and then is administered additional doses (maintenance doses) of from about 5 mg/kg to about 10 mg/kg every three weeks.

In some embodiments, the activatable anti-CTLA4 antibody is administered at a dose of about 20 mg/kg for one treatment cycle and at a dose of about 6 mg/kg in subsequent treatment cycles (e.g., once every three weeks or once every six weeks). For instance, in one embodiment, the activatable anti-CTLA4 antibody is administered at an initial (loading) dose of 6 mg/kg and then is administered additional doses (maintenance doses) of 6 mg/kg every three weeks. In some embodiments, the activatable anti-CTLA4 antibody is administered at a dose of about 20 mg/kg for two treatment cycles and at a dose of about 6 mg/kg in subsequence treatment cycles (e.g., once every three weeks). In one embodiment, the activatable anti-CTLA4 antibody is administered at an initial (loading) dose of 20 mg/kg, administered another loading dose of 20 mg/kg three weeks later, and then is administered additional doses (maintenance doses) of 6 mg/kg every three weeks.

In some embodiments, the activatable anti-CTLA4 antibody is administered at a dose of about 20 mg/kg for one treatment cycle and at a dose of about 10 mg/kg in subsequent treatment cycles (e.g., once every three weeks or once every six weeks). For instance, in one embodiment, the activatable anti-CTLA4 antibody is administered at an initial (loading) dose of 20 mg/kg and then is administered additional doses (maintenance doses) of 10 mg/kg every three weeks. In some embodiments, the activatable anti-CTLA4 antibody is administered at a dose of about 20 mg/kg for two treatment cycles and at a dose of about 10 mg/kg in subsequence treatment cycles (e.g., once every three weeks). In one embodiment, the activatable anti-CTLA4 antibody is administered at an initial (loading) dose of 20 mg/kg, administered another loading dose of 20 mg/kg three weeks later, and then is administered additional doses (maintenance doses) of 10 mg/kg every three weeks.

In one embodiment, the activatable anti-CTLA4 antibody is administered at a dose of from about 30 mg/kg to about 50 mg/kg (e.g., 30 mg/kg, 35 mg/kg, 40 mg/kg, 45 mg/kg or 50 mg/kg) for at least one treatment cycle (e.g., one to three treatment cycles) and at a dose of about 5 mg/kg in subsequent treatment cycles (e.g., once every three weeks or once every six weeks). In one embodiment, the activatable anti-CTLA4 antibody is administered at a dose of from about 30 mg/kg to about 50 mg/kg (e.g., 30 mg/kg, 35 mg/kg, 40 mg/kg, 45 mg/kg or 50 mg/kg) for at least one treatment cycle (e.g., one to three treatment cycles) and at a dose of about 6 mg/kg in subsequent treatment cycles (e.g., once every three weeks or once every six weeks). In one embodiment, the activatable anti-CTLA4 antibody is administered at a dose of from about 30 mg/kg to about 50 mg/kg (e.g., 30 mg/kg, 35 mg/kg, 40 mg/kg, 45 mg/kg or 50 mg/kg) for at least one treatment cycle (e.g., one to three treatment cycles) and at a dose of about 10 mg/kg in subsequent treatment cycles (e.g., once every three weeks or once every six weeks). In another embodiment, the activatable anti-CTLA4 antibody is administered at a dose of from about 30 mg/kg to about 50 mg/kg (e.g., 30 mg/kg, 35 mg/kg, 40 mg/kg, 45 mg/kg or 50 mg/kg) for at least one treatment cycle (e.g., one to three treatment cycles) and at a dose of about 15 mg/kg in subsequent treatment cycles (e.g., once every three weeks or once every six weeks). In another embodiment, the activatable anti-CTLA4 antibody is administered at a dose of from about 30 mg/kg to about 50 mg/kg (e.g., 30 mg/kg, 35 mg/kg, 40 mg/kg, 45 mg/kg or 50 mg/kg) for at least one treatment cycle (e.g., one to three treatment cycles) and at a dose of about 20 mg/kg in subsequent treatment cycles (e.g., once every three weeks or once every six weeks).

In some embodiments, the activatable anti-CTLA4 antibody is administered at a dose ranging from about 30 to about 50 mg/kg for one treatment cycle and at a dose of about 6 mg/kg in subsequent treatment cycles (e.g., once every three weeks). For instance, in one embodiment, the activatable anti-CTLA4 antibody is administered at an initial (loading) dose of 30 mg/kg and then is administered additional doses (maintenance doses) of 6 mg/kg every three weeks. In one embodiment, the activatable anti-CTLA4 antibody is administered at an initial (loading) dose of 40 mg/kg and then is administered additional doses (maintenance doses) of 6 mg/kg every three weeks. In one embodiment, the activatable anti-CTLA4 antibody is administered at an initial (loading) dose of 50 mg/kg and then is administered additional doses (maintenance doses) of 6 mg/kg every three weeks. The start of the maintenance doses can be at a predetermined time following administration of the loading dose. For instance, in some embodiments, the first maintenance dose can be administered three weeks after administration of the loading dose.

In some embodiments, the activatable anti-CTLA4 antibody is administered at a dose ranging from about 30 to about 50 mg/kg for one treatment cycle and at a dose of about 10 mg/kg in subsequent treatment cycles (e.g., once every three weeks). For instance, in one embodiment, the activatable anti-CTLA4 antibody is administered at an initial (loading) dose of 30 mg/kg and then is administered additional doses (maintenance doses) of 10 mg/kg every three weeks. In one embodiment, the activatable anti-CTLA4 antibody is administered at an initial (loading) dose of 40 mg/kg and then is administered additional doses (maintenance doses) of 10 mg/kg every three weeks. In one embodiment, the activatable anti-CTLA4 antibody is administered at an initial (loading) dose of 50 mg/kg and then is administered additional doses (maintenance doses) of 10 mg/kg every three weeks. The start of the maintenance doses can be at a predetermined time following administration of the loading dose. For instance, in some embodiments, the first maintenance dose can be administered three weeks after administration of the loading dose.

In some embodiments, the activatable anti-CTLA4 antibody is administered at a dose ranging from about 40 to about 50 mg/kg for one treatment cycle and at a dose of about 30 mg/kg in subsequent treatment cycles (e.g., once every three weeks). For instance, in one embodiment, the activatable anti-CTLA4 antibody is administered at an initial (loading) dose of 40 mg/kg and then is administered additional doses (maintenance doses) of 30 mg/kg every three weeks. In one embodiment, the activatable anti-CTLA4 antibody is administered at an initial (loading) dose of 40 mg/kg and then is administered additional doses (maintenance doses) of 30 mg/kg every three weeks. In one embodiment, the activatable anti-CTLA4 antibody is administered at an initial (loading) dose of 50 mg/kg and then is administered additional doses (maintenance doses) of 30 mg/kg every three weeks. The start of the maintenance doses can be at a predetermined time following administration of the loading dose. For instance, in some embodiments, the first maintenance dose can be administered three weeks after administration of the loading dose.

It has been found that administration of a single loading dose followed by subsequent administration of maintenance doses, as set forth above, results in establishing a steady-state plasma concentration of the cleaved antibody more rapidly than when the activatable antibody is administered without a loading dose. It has been found that administration of two loading doses followed by subsequent administration of maintenance doses, as set forth above, results in establishing a steady-state plasma concentration of the cleaved antibody more rapidly than when the activatable antibody is administered without a loading dose. For instance, in some embodiments, a steady-state plasma concentration of the cleaved antibody can be established within 1 week, within 2 weeks, within 3 week, within 4 weeks, within 5 week, within 6 weeks or within 7 weeks following the initial administration of the loading dose or loading doses.

In any of the foregoing embodiments, the activatable anti-CTLA4 antibody (e.g., TY22404), when administered in combination with pembrolizumab, can be administered at a dose that provides a steady-state plasma concentration of the cleaved antibody (i.e., active antibody following cleavage of the masking moiety (MM) and cleavable moiety (CM)) of from about 50 nM to about 100 nM. In some embodiments, the activatable anti-CTLA4 antibody is administered at a dose that provides a steady-state plasma concentration of the cleaved antibody of from about 50 nM to about 100 nM. In some embodiments, the activatable anti-CTLA4 antibody is administered at a dose that provides a steady—state plasma concentration of the cleaved antibody of from about 50 nM to about 75 nM. In some embodiments, the activatable anti-CTLA4 antibody is administered at a dose that provides a steady—state plasma concentration of the cleaved antibody of from about 70 nM to about 80 nM. In some embodiments, the activatable anti-CTLA4 antibody is administered at a dose that provides a steady-state plasma concentration of the cleaved antibody of from about 100 nM to about 175 nM. In some embodiments, the activatable anti-CTLA4 antibody is administered at a dose that provides a steady-state plasma concentration of the cleaved antibody of from about 100 nM to about 200 nM. In some embodiments, the activatable anti-CTLA4 antibody is administered at a dose that provides a steady-state plasma concentration of the cleaved antibody of from about 125 nM to about 200 nM. In some embodiments, the activatable anti-CTLA4 antibody is administered at a dose that provides a steady-state plasma concentration of the cleaved antibody of from about 100 nM to about 150 nM. In some embodiments, the activatable anti-CTLA4 antibody is administered at a dose that provides a steady-state plasma concentration of the cleaved antibody of from about 125 nM to about 175 nM. In some embodiments, the activatable anti-CTLA4 antibody is administered at a dose that provides a steady-state plasma concentration of the cleaved antibody of from about 125 nM to about 150 nM. In some embodiments, the activatable anti-CTLA4 antibody is administered at a dose that provides a steady-state plasma concentration of the cleaved antibody of from about 150 nM to about 200 nM. In some embodiments, the activatable anti-CTLA4 antibody is administered at a dose that provides a steady-state plasma concentration of the cleaved antibody of from about 200 nM to about 600 nM. In some embodiments, the activatable anti-CTLA4 antibody is administered at a dose that provides a steady-state plasma of the cleaved antibody of from about 200 nM to about 400 nM. In some embodiments, the activatable anti-CTLA4 antibody is administered at a dose that provides a steady-state plasma concentration of the cleaved antibody of from about 300 nM to about 500 nM. In some embodiments, the activatable anti-CTLA4 antibody is administered at a dose that provides a steady-state plasma concentration of the cleaved antibody of from about 400 nM to about 600 nM. In some of the foregoing embodiments, the plasma concentration can be measured at the trough level of the activatable anti-CTLA4 antibody (i.e., minimal concentration of each dosing cycle). For instance, the plasma concentration of a particular cycle can be measured immediately prior to administering a dose at the next cycle.

In some embodiments, the activatable anti-CTLA4 antibody and pembrolizumab is administered in combination with two or more therapeutic agents. In some such embodiments, the activatable anti-CTLA4 antibody is administered at a dose that provides a steady-state plasma concentration of the cleaved antibody of from about 50 nM to about 150 nM. In other such embodiments, the activatable anti-CTLA4 antibody is administered at a dose that provides a steady-state plasma concentration of the cleaved antibody of from about 50 nM to about 100 nM. In other such embodiments, the activatable anti-CTLA4 antibody is administered at a dose that provides a steady-state plasma concentration of the cleaved antibody of from about 50 nM to about 75 nM. In other such embodiments, the activatable anti-CTLA4 antibody is administered at a dose that provides a steady-state plasma concentration of the cleaved antibody of from about 75 nM to about 100 nM.

In some embodiments, the cancer is resistant or refractory to an inhibitor of CTLA-4, PD-1 or a PD-1 ligand (e.g., PD-L1 or PD-L2). In some embodiments, the cancer is a solid cancer, such as advanced-stage and/or metastatic Cancer treatments can be evaluated by, e.g., tumor regression, tumor weight or size shrinkage, time to progression, duration of survival, progression free survival, overall response rate, duration of response, quality of life, protein expression and/or activity. Approaches to determining efficacy of therapy can be employed, including for example, measurement of response through radiological imaging.

The activatable anti-CTLA4 antibodies and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The antibodies and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art. In some embodiments, the activatable anti-CTLA4 antibody is administered intravenously.

The effective amount of the activatable anti-CTLA4 antibody may be administered in a single dose or in multiple doses. For methods that comprise administration of the activatable anti-CTLA4 antibody in multiple doses, exemplary dosing frequencies include, but are not limited to weekly, weekly without break, weekly for two out of three weeks, weekly for three out of four weeks, once every three weeks, once every two weeks, monthly, every six months, yearly, etc. In some embodiments, the activatable anti-CTLA4 antibody is administered about weekly, once every 2 weeks, once every 3 weeks, once every 6 weeks, or once every 12 weeks. In some embodiments, the intervals between each administration are less than about any of 3 years, 2 years, 12 months, 11 months, 10 months, 9 months, 8 months, 7 months, 6 months, 5 months, 4 months, 3 months, 2 months, 1 month, 4 weeks, 3 weeks, 2 weeks, or 1 week. In some embodiments, the intervals between each administration are more than about any of 1 week, 2 weeks, 3 weeks, 4 weeks, 1 month, 2 months, 3 months, 4 months, 5 months, 6 months, 7 months, 8 months, 9 months, 10 months, 11 months, 12 months, 2 years, or 3 years. In some embodiments, there is no break in the dosing schedule.

In some embodiments, the activatable anti-CTLA4 antibody is administered at a low frequency, for example, any one of no more frequent than once per week, once every other week, once per three weeks, once per month, once per 2 months, once per 3 months, once per 4 months, once per 5 months, once per 6 months, once per 7 months, once per 8 months, once per 9 months, once per 10 months, once per 11 months, once per year, or less. In some embodiments, the activatable anti-CTLA4 antibody is administered in a single dose. In some embodiments, the activatable anti-CTLA4 antibody is administered about once every three weeks. In some embodiments, the activatable anti-CTLA4 antibody is administered about once every six weeks.

In some embodiments, the activatable anti-CTLA4 antibody is administered for 2 or more cycles, such as about any one of 2, 3, 4, 5, 6, 7, 8, 9, 10, 11, 12, or more cycles. In some embodiments, the activatable anti-CTLA4 antibody is administered for at least 4 cycles.

The activatable anti-CTLA4 antibodies can be administered to patients in combination with pembrolizumab at doses that achieve high levels of receptor (CTLA-4) occupancy, and hence are efficacious while at the same time having minimal side effects. Hence, the activatable anti-CTLA4 antibodies of the disclosure show improved therapeutic indexes relative to anti-CTLA4 antibodies such as Ipilimumab. For instance, in one embodiment, the activatable antibody (e.g., TY22404) can be administered as a single dose (in combination with pembrolizumab) that achieves greater than 50% receptor occupancy three weeks or even six weeks following administration. In some such embodiments, the activatable anti-CTLA4 antibody (e.g., TY22404) can be administered as a single dose (in combination with pembrolizumab) that achieves greater than 60% receptor occupancy three weeks following administration. In other such embodiments, the activatable anti-CTLA4 antibody (e.g., TY22404) can be administered as a single dose (in combination with pembrolizumab) that achieves greater than 70% receptor occupancy three weeks following administration. In other such embodiments, the activatable anti-CTLA4 antibody (e.g., TY22404) can be administered as a single dose (in combination with pembrolizumab) that achieves greater than 80% receptor occupancy three weeks following administration. In other such embodiments, the activatable anti-CTLA4 antibody (e.g., TY22404) can be administered as a single dose (in combination with pembrolizumab) that achieves from about 50% to about 80% receptor occupancy three weeks following administration. In other such embodiments, the activatable anti-CTLA4 (e.g., TY22404) antibody can be administered as a single dose (in combination with pembrolizumab) that achieves from about 60% to about 75% receptor occupancy three weeks following administration. In other such embodiments, the activatable anti-CTLA4 antibody (e.g., TY22404) can be administered as a single dose (in combination with pembrolizumab) that achieves greater than 60% receptor occupancy six weeks following administration. In other such embodiments, the activatable anti-CTLA4 antibody (e.g., TY22404) can be administered as a single dose (in combination with pembrolizumab) that achieves greater than 70% receptor occupancy six weeks following administration. In other such embodiments, the activatable anti-CTLA4 antibody (e.g., TY22404) can be administered as a single dose (in combination with pembrolizumab) that achieves from about 50% to about 70% receptor occupancy six weeks following administration.

In some embodiments, the treatment comprises an initial phase and a subsequent maintenance phase. In some embodiments, the activatable anti-CTLA4 antibody (e.g., TY22404) is administered less frequently in the maintenance phase than in the initial phase. In some embodiments, the activatable anti-CTLA4 antibody is administered at the same frequently in the maintenance phase as in the initial phase. In some embodiments, the treatment comprises an initial phase wherein the activatable anti-CTLA4 antibody is administered about once every three weeks for at least 4 cycles, and a maintenance phase wherein the activatable anti-CTLA4 antibody is administered about once every 4 weeks to once every 12 weeks, such as once every 4 weeks, once every 6 weeks, once every 8 weeks, once every 10 weeks, or once every 12 weeks. In some embodiments, the dosing frequency in the maintenance phase is adjusted depending on one or more biomarkers, such as T_{reg} cells, CD8+ Tₑₘ cells, CD4+ Tₑₘ cells, a ratio of CD8+ Tₑₘ cells to T_{reg} cells, a ratio of CD4+ Tₑₘ cells to T_{reg} cells, and/or NK cells. For example, if the subject (e.g., a human patient) shows an increase in the ratio of CD8+ Tₑₘ cells to T_{reg} cells after receiving the anti-CTLA4 antibody, the subject (e.g., a human patient) may be further administered an activatable anti-CTLA4 antibody at about every 4 weeks.

The administration of the activatable anti-CTLA4 antibody in combination with pembrolizumab can be extended over an extended period of time, such as from about a week to about a month, from about a month to about a year, from about a year to about several years. In some embodiments, the activatable anti-CTLA4 antibody is administered over a period of at least any of about 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more.

The methods described herein are useful for treating a variety of cancers. In some embodiments, the cancer is a solid cancer. In some embodiments, the cancer is a liquid cancer. A variety of cancers where CTLA4 is implicated, whether malignant or benign and whether primary or secondary, may be treated or prevented with a method provided by the disclosure. Exemplary cancers include, but are not limited to, liver cancer, a cancer of the digestive system (e.g., colon cancer, colorectal cancer), lung cancer, bone cancer, heart cancer, brain cancer, kidney cancer, bladder cancer, a hematological cancer (e.g., leukemia), skin cancer, breast cancer, thyroid cancer, pancreatic cancer, a head and/or neck cancer, an eye-related cancer, a male reproductive system cancer (e.g., prostate cancer, testicular cancer), or a female reproductive system cancer (e.g., uterine cancer, cervical cancer). In some embodiments, the cancer is kidney cancer, such as renal cell carcinoma, or urothelial carcinoma. In some embodiments, the cancer is a cold tumor. In some embodiments, the cancer is resistant or refractory to one or more prior therapies, such as immunotherapies, including immune checkpoint inhibitor(s). In some embodiments, the cancer is a tumor that T cells cannot penetrate because the tumor has not been recognized by the immune system, or provoked an immune response.

In some embodiments, the activatable anti-CTLA4 antibodies of the disclosure (in combination with pembrolizumab) can be used to treat colorectal cancer (CRC). In some embodiments, the colorectal cancer has not metastasized to other organs, such as the lung or the liver. In some embodiments, the colorectal cancer has metastasized to other organs, such as the lung or the liver. In some embodiments, the colorectal cancer patient had previously been treated with other chemotherapeutic reagents. Such chemotherapeutic reagents include, but are not limited to, FOLFOX, FOLFIRI/Avastin, Erbitux, Lonsurf, IO-202, APN401, or IPH5201.

In some embodiments, the activatable anti-CTLA4 antibodies of the disclosure (in combination with pembrolizumab) can be used to treat colorectal cancer (CRC), wherein the CRC is microsatellite stable (MSS)-colorectal cancer (MSS CRC). In some embodiments, the MSS CRC has metastasized to other organs, such as the lung or the liver. In some embodiments, the MSS CRC has not metastasized to other organs, such as the lung or the liver. In some embodiments, the MSS CRC has not metastasized to the peritoneum. In some embodiments, the MSS CRC has not metastasized to either the liver or to the peritoneum. In some embodiments, the MSS CRC patient had previously been treated with other chemotherapeutic reagents. Such chemotherapeutic reagents include, but are not limited to, FOLFOX, FOLFIRI/Avastin, Erbitux, Lonsurf, IO-202, APN401, or IPH5201.

In some embodiments, the activatable anti-CTLA4 antibodies of the disclosure can be used to treat Kaposi's sarcoma.

In some embodiments, the activatable anti-CTLA4 antibodies of the disclosure (in combination with pembrolizumab) can be used to treat head and neck squamous cell carcinoma (HNSCC).

In some embodiments, the anti-CTLA4 antibodies of the disclosure (in combination with pembrolizumab) can be used to treat pancreatic cancer.

In some embodiments, the anti-CTLA4 antibodies of the disclosure (in combination with pembrolizumab) can be used to treat ovarian cancer.

In some embodiments, the subject (e.g., a human patient) has been previously treated with a prior therapy. In some embodiments, the subject (e.g., a human patient) has previously received any one of 1, 2, 3, 4, or more prior therapies. In some embodiments, the subject (e.g., a human patient) has exhausted all other available therapies. In some embodiments, the subject (e.g., a human patient) is unresponsive or resistant to a prior therapy. In some embodiments, the subject (e.g., a human patient) has disease reoccurrence subsequent to a prior therapy. In some embodiments, the subject (e.g., a human patient) is refractory to a prior therapy. In some embodiments, the subject (e.g., a human patient) has failed a prior therapy within about 1 year, 6 months, 3 months or less. In some embodiments, the subject (e.g., a human patient) has not previously received a prior therapy.

In some embodiments, the subject (e.g., a human patient) has been previously treated with a standard therapy for the cancer. In some embodiments, the subject (e.g., a human patient) is unresponsive or resistant to a standard therapy. In some embodiments, the subject (e.g., a human patient) has disease reoccurrence subsequent to a standard therapy. In some embodiments, the subject (e.g., a human patient) is refractory to a standard therapy. In some embodiments, the subject (e.g., a human patient) has failed a standard therapy within about 1 year, 6 months, 3 months or less. In some embodiments, the subject (e.g., a human patient) has not previously received a standard therapy. In some embodiments, the subject (e.g., a human patient) has refused or is ineligible for a standard therapy.

In some embodiments, the prior therapy (e.g., standard therapy) is selected from the group consisting of viral gene therapy, immunotherapy, targeted therapy, radiation therapy, and chemotherapy. In some embodiments, the prior therapy is an immune checkpoint inhibitor. In some embodiments, the prior therapy is an inhibitor of CTLA4, PD-1, or a PD-1 ligand (e.g., PD-L1 or PD-L2). In some embodiments, the prior therapy is an inhibitor of CTLA4, such as an anti-CTLA4 antibody that is different from the anti-CTLA4 antibodies described herein. In some embodiments, the prior therapy is ipilimumab.

In some embodiments, the prior therapy is an inhibitor of PD-1 or a PD-1 ligand, including a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the prior therapy is an inhibitor of PD-1 that is a molecule that inhibits the binding of PD-1 to its ligand binding partners. In some embodiments, the inhibitor of a PD-1 ligand is an inhibitor of PD-L1 and/or PD-L2. In some embodiments, the inhibitor of PD-L1 is a molecule that inhibits the binding of PDL1 to its binding partners. In some embodiments, a PD-L2 binding partner is PD-1 and/or B7-1. In some embodiments, the inhibitor of a PD-1 ligand is a molecule that inhibits the binding of PD-L2 to its binding partners. In some embodiments, a PD-L2 binding partner is PD-1. The inhibitor may be an antibody, an antigen binding fragment thereof, an immunoadhesin, a fusion protein, or oligopeptide.

In some embodiments, the prior therapy is an anti-PD-1 antibody selected from pembrolizumab, 2E5 (Cstone Pharmaceuticals), tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), PF-06801591, cemiplimab (REGN-2810, REGEN2810), dostarlimab (TSR-042, ANB011), pidilizumab (CT-011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, CCX-4503, biosimilars thereof, or derivatives thereof. In some embodiments, the anti-PD-1 antibody is selected from the group consisting of nivolumab, and CT-011. In some embodiments, the inhibitor of PD-1 is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the inhibitor of PD-1 is AMP-224. In some embodiments, the anti-PD-1 antibody is nivolumab (CAS Registry Number: 946414-94-4). Nivolumab, also known as MDX-1106-04, MDX-1106, ONO-4538, BMS-936558, and OPDIVO®, is an anti-PD-1 antibody described in WO2006/121168. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Prior therapies (e.g., standard therapies) also encompass surgery to remove a tumor and radiation therapy. Exemplary radiation therapies include, but are not limited to, ionizing (electromagnetic) radiotherapy (e.g., X-rays or gamma rays) and particle beam radiation therapy (e.g., high linear energy radiation). The source of radiation can be external or internal to the subject (e.g., a human patient).

The methods described herein are useful for various aspects of cancer treatment. In some embodiments, there is provided a method of inhibiting cell proliferation (such as tumor growth) in an individual, comprising administering to the individual an effective amount of an activatable anti-CTLA4 antibody of the disclosure in combination with pembrolizumab. In some embodiments, at least about 10% (including for example at least about any of 20%, 30%, 40%, 60%, 70%, 80%, 90%, 95% or more) cell proliferation is inhibited.

In some embodiments, administration of pembrolizumab in combination with an activatable antibody of the disclosure (e.g., TY22404) significantly increases levels of IFN-γ relative to monotherapy with an activatable antibody of the disclosure (e.g., TY22404). For instance, in some embodiments, administration of pembrolizumab in combination with an activatable antibody of the disclosure (e.g., TY22404) at a particular dose (e.g., 6 mg/kg or 10 mg/kg) results in a 2-fold to 10-fold increase of IFN-γ relative to administration of the activatable anti-CTLA4 monotherapy administered at the same dose.

In some embodiments, there is provided a method of inhibiting tumor metastasis in an individual, comprising administering to the individual an effective amount of any one of the activatable anti-CTLA4 antibodies described herein in combination with pembrolizumab. In some embodiments, at least about 10% (including for example at least about any of 20%, 30%, 40%, 60%, 70%, 80%, 90%, 95% or more) metastasis is inhibited.

In some embodiments, there is provided a method of reducing (such as eradicating) pre-existing tumor metastasis (such as metastasis to the lymph node) in an individual, comprising administering to the individual an effective amount of any one of the activatable anti-CTLA4 antibodies described herein in combination with pembrolizumab. In some embodiments, at least about 10% (including for example at least about any of 20%, 30%, 40%, 60%, 70%, 80%, 90%, 95% or more) metastasis is reduced.

In some embodiments, there is provided a method of reducing incidence or burden of preexisting tumor metastasis (such as metastasis to the lymph node) in an individual, comprising administering to the individual an effective amount of any one of the activatable anti-CTLA4 antibodies described herein in combination with pembrolizumab.

In some embodiments, there is provided a method of reducing tumor size in an individual, comprising administering to the individual an effective amount of any one of the activatable anti-CTLA4 antibodies described herein in combination with pembrolizumab. In some embodiments, the method reduces tumor size by at least about 10% (including for example at least about any of 20%, 30%, 40%, 60%, 70%, 80%, 90%, 95% or more).

In some embodiments, there is provided a method of prolonging time to disease progression of cancer in an individual, comprising administering to the individual an effective amount of any one of the activatable anti-CTLA4 antibodies described herein in combination with pembrolizumab. In some embodiments, the method prolongs the time to disease progression by at least any of 1, 2, 3, 4, 5, 6, 7, 8, 9, 10, 11, 12, 16, 20, 24, 28, 32, 36, or more weeks.

In some embodiments, there is provided a method of prolonging survival (e.g., overall survival or progression-free survival) of an individual having cancer, comprising administering to the individual an effective amount of any one of the activatable anti-CTLA4 antibodies described herein in combination with pembrolizumab. In some embodiments, the method prolongs the survival of the individual by at least any of 1, 2, 3, 4, 5, 6, 7, 8, 9, 10, 11, 12, 18, or 24 months.

In some embodiments, there is provided a method of alleviating one or more symptoms in an individual having cancer, comprising administering to the individual an effective amount of any one of the activatable anti-CTLA4 antibodies described herein in combination with pembrolizumab.

In some embodiments, there is provided a method of improving the quality of life in an individual having cancer, comprising administering to the individual an effective amount of any one of the activatable anti-CTLA4 antibodies described herein in combination with pembrolizumab.

The activatable anti-CTLA4 antibody and pembrolizumab may be in combination with one or more additional therapeutic agents or therapies. In some embodiment, the activatable anti-CTLA4 antibody and pembrolizumab is administered in combination with one or more additional therapeutic agents for separate, sequential or simultaneous administration. The term “additional therapeutic agent” refers to any therapeutic agent other than an activatable anti-CTLA4 antibody provided by the disclosure. In some embodiments, there is provided a combination therapy for treating cancer in a subject (e.g., a human patient), which comprises administering to the subject (e.g., a human patient) a therapeutically effective amount of an activatable anti-CTLA4 antibody described herein in combination with one or more additional therapeutic agents. In some embodiments, activatable anti-CTLA4 antibody is administered in combination with one or more additional therapeutic agents comprising chemotherapeutic agents, immunotherapeutic agents, and/or hormone therapeutic agents. In some embodiments, the one or more additional therapeutic agents are selected from the group consisting of selected from the group consisting of viral gene therapy, immune checkpoint inhibitors, targeted therapies, radiation therapies, and chemotherapies.

### III. Activatable Anti-CTLA4 Antibodies

The present disclosure also relates, in part, to precision/context-dependent activatable binding polypeptides (i.e., activatable antibodies) that bind to human CTLA4 to be administered in combination with pembrolizumab. The activatable antibodies comprise any of the anti-CTLA4 antibodies described herein (e.g., anti-CTLA4 antibodies, anti-CTLA4 antibody binding fragments, and/or anti-CTLA4 antibody derivatives), antigen binding fragments of the activatable anti-CTLA4 antibodies, and/or derivatives of the activatable anti-CTLA4 antibodies. In some embodiments, the activatable anti-CTLA4 antibodies described herein may have improved safety profiles. For example, the activatable anti-CTLA4 antibodies described herein may have better safety margin as assessed by spleen weight change. The change in spleen size with the increase in drug dose administered is used as a benchmark to assess the safety margin of the drug candidate used. The activatable anti-CTLA4 antibodies described herein have a better safety margin relative to the parental antibody (the antibody without the masking moiety). In some embodiments, the activatable antibody is TY22404.

In some embodiments, an activatable antibody of the present disclosure comprises: (a) a masking moiety (MM); (b) a cleavable moiety (CM); and (c) a target binding moiety (e.g., anti-CTLA4 antibody). In some embodiments, the MM is any of the masking moieties described herein. In some embodiments, the CM is any of the cleavable moieties described herein. In some embodiments, the TBM is any of the target binding moieties described herein (e.g., a target binding moiety (TBM) comprising an anti-CTLA4 antibody light chain variable region and/or an antibody heavy chain variable region, such as a VH and/or VL of any of the anti-CTLA4 antibodies described herein).

In some embodiments, the activatable antibody comprises: (a) a polypeptide comprising, from N-terminus to C-terminus, a masking moiety (MM), a cleavable moiety (CM), and a target binding moiety (TBM), where the MM is any of the masking moieties described herein, the CM is any of the cleavable moieties described herein, and where the TBM comprises an anti-CTLA4 antibody light chain variable region (VL); and (b) an anti-CTLA4 antibody heavy chain variable region (VH).

In some embodiments, the activatable antibody comprises: (a) a polypeptide comprising, from N-terminus to C-terminus, a masking moiety (MM), a cleavable moiety (CM), and a target binding moiety (TBM), where the MM is any of the masking moieties described herein, the CM is any of the cleavable moieties described herein, and where the TBM comprises an anti-CTLA4 antibody heavy chain variable region (VH); and (b) an anti-CTLA4 antibody light chain variable region (VL).

In some embodiments, the activatable antibody comprises: a polypeptide comprising, from N-terminus to C-terminus, a masking moiety (MM), a cleavable moiety (CM), and a target binding moiety (TBM), where the MM is any of the masking moieties described herein, the CM is any of the cleavable moieties described herein, and where the TBM comprises an antibody heavy chain variable region (VH) and an antibody light chain variable region (VL).

The term “activatable binding polypeptide”, “ABP”, or “activatable antibody” includes a polypeptide that comprises a target binding moiety (TBM), a cleavable moiety (CM), and a masking moiety (MM). In some embodiments, the TBM comprises an amino acid sequence that binds to a target. In some embodiments, the TBM (anti-CTLA4 antibody) comprises an antigen binding domain (ABD) of an antibody or antibody fragment thereof (e.g., any of the antibodies or antigen binding fragments described herein).

### Anti-CTLA4 Antibodies

The method described herein comprise administration of an activatable anti-CTLA4 antibody that specifically binds to human CTLA4, including CTLA4 antibodies, antigen-binding fragments of the CTLA4 antibodies, and derivatives of the CTLA4 antibodies. Exemplary anti-CTLA4 antibodies have been described, for example, in International Publication No. WO2019149281A1, which is incorporated herein by reference in its entirety.

In some embodiments, the activatable anti-CTLA4 antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), and an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75).

In some such embodiments, the activatable anti-CTLA4 antibody, upon cleavage of the CM, comprises: a) a heavy chain variable region comprising the amino acid sequence of SEQ ID NO: 87, and a light chain variable region comprising the amino acid sequence of SEQ ID NO: 100. In some embodiments, the activatable anti-CTLA4 antibody comprises a heavy chain variable region comprising an amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 87, and/or a light chain variable region comprising the amino acid sequence having at least 90% (e.g., at least 92%, at least 93%, at least 94%, at least 95%, at least 96%, at least 97%, at least 98% or at least 99%) sequence identity to the amino acid sequence of SEQ ID NO: 100.

In some embodiments, the activatable anti-CTLA4 antibody comprises a heavy chain comprising the amino acid sequence of SEQ ID NO:320 and a light chain comprising the amino acid sequence of SEQ ID NO:322. The activatable antibody having heavy chain SEQ ID No: 320 and light chain SEQ ID No. 322 is referred to as TY22404. In some embodiments, the activatable antibody comprises a heavy chain comprising the amino acid sequence of SEQ ID NO: 320 and a light chain comprising the amino acid sequence of SEQ ID NO:322. In some embodiments, the activatable anti-CTLA4 antibody comprises a heavy chain having at least 90% (e.g., at least 92%, at least 93%, at least 94%, at least 95%, at least 96%, at least 97%, at least 98% or at least 99%) sequence identity to the amino acid sequence of SEQ ID NO:320. In some embodiments, the activatable anti-CTLA4 antibody comprises a light chain having at least 90% (e.g., at least 92%, at least 93%, at least 94%, at least 95%, at least 96%, at least 97%, at least 98% or at least 99%) sequence identity to the amino acid sequence of SEQ ID NO:321. In some embodiments, the activatable anti-CTLA4 antibody comprises a heavy chain having at least 90% (e.g., at least 92%, at least 93%, at least 94%, at least 95%, at least 96%, at least 97%, at least 98% or at least 99%) sequence identity to the amino acid sequence of SEQ ID NO:320. In some embodiments, the activatable anti-CTLA4 antibody comprises a light chain having at least 90% (e.g., at least 92%, at least 93%, at least 94%, at least 95%, at least 96%, at least 97%, at least 98% or at least 99%) sequence identity to the amino acid sequence of SEQ ID NO:322. In some embodiments, the activatable antibody is TY22404.

In some embodiments, the activatable antibody comprises a polypeptide comprising the structure, from N-terminus to C-terminus, of: masking moiety (MM)-cleavable moiety (CM)-VL, and the activatable antibody further comprises a second polypeptide comprising a VH (e.g., a Fab fragment). In some embodiments, the activatable antibody comprises a polypeptide comprising the structure, from N-terminus to C-terminus, of: masking moiety (MM)-cleavable moiety (CM)-VL-VH (e.g., an scFv). In some embodiments, the activatable antibody comprises a polypeptide comprising the structure, from N-terminus to C-terminus, of: masking moiety (MM)-cleavable moiety (CM)-VH, and the activatable antibody further comprises a second polypeptide comprising a VL (e.g., a Fab fragment). In some embodiments, the activatable antibody comprises a polypeptide comprising the structure, from N-terminus to C-terminus, of: masking moiety (MM)-cleavable moiety (CM)-VH-VL (e.g., an scFv).

The CM generally includes an amino acid sequence that is cleavable, for example, serves as the substrate for an enzyme and/or a cysteine-cysteine pair capable of forming a reducible disulfide bond. As such, when the terms “cleavage,” “cleavable,” “cleaved” and the like are used in connection with a CM, the terms encompass enzymatic cleavage, e.g., by a protease, as well as disruption of a disulfide bond between a cysteine-cysteine pair via reduction of the disulfide bond that can result from exposure to a reducing agent.

The MM refers to an amino acid sequence that, when the CM of the activatable antibody is intact (e.g., uncleaved by a corresponding enzyme, and/or containing an unreduced cysteine-cysteine disulfide bond), the MM interferes with or inhibits binding of the TBM to its target. In some embodiments, the MM interferes with or inhibits binding of the TBM to its target so efficiently that binding of the TBM to its target is extremely low and/or below the limit of detection (e.g., binding cannot be detected in an ELISA or flow cytometry assay). The amino acid sequence of the CM may overlap with or be included within the MM. It should be noted that for sake of convenience “ABP” or “activatable antibody” are used herein to refer to an ABP or activatable antibody in both their uncleaved (or “native”) state, as well as in their cleaved state. It will be apparent to the ordinarily skilled artisan that in some embodiments a cleaved ABP may lack an MM due to cleavage of the CM, e.g., by a protease, resulting in release of at least the MM (e.g., where the MM is not joined to the ABP by a covalent bond (e.g., a disulfide bond between cysteine residues)). Exemplary ABPs are described in more detail below.

In some embodiments, the masking moiety (MM) interferes with, obstructs, reduces the ability of, prevents, inhibits, or competes with the target binding moiety for binding to its target (e.g., an “inactive activatable antibody). In some embodiments, the masking moiety (MM) interferes with, obstructs, reduces, prevents, inhibits, or competes with the target binding moiety for binding to its target only when the polypeptide has not been activated (e.g., activated by a change in pH (increased or decreased), activated by a temperature shift (increased or decreased), activated after being contacted with a second molecule (such as a small molecule or a protein ligand), etc.). In some embodiments, activation induces cleavage of the polypeptide within the cleavage moiety. In some embodiments, activation induces conformation changes in the polypeptide (e.g., displacement of the masking moiety (MM)), leading to the masking moiety no longer preventing the activatable antibody from binding to its target. In some embodiments, the masking moiety (MM) interferes with, obstructs, reduces the ability of, prevents, inhibits, or competes with the target binding moiety for binding to its target only when the cleavable moiety (CM) has not been cleaved by one or more proteases that cleave within the cleavable moiety (CM). In some embodiments, the masking moiety (MM) has a masking efficiency of at least about 2.0 (e.g., at least about 2.0, at least about 3.0, at least about 4.0, at least about 5.0, at least about 6.0, at least about 7.0, at least about 8.0, at least about 9.0, at least about 10, at least about 25, at least about 50, at least about 75, at least about 100, at least about 150, at least about 200, at least about 300, at least about 400, at least about 500, etc.) prior to activation. In some embodiments, masking efficiency is measured as the difference in affinity of an activatable antibody comprising the masking moiety (MM) for binding its target (before activation) relative to the affinity of a polypeptide lacking the masking moiety for binding its target (e.g., the difference in affinity for a target antigen (such as CTLA4) of an activatable antibody comprising a masking moiety (MM) (before activation) relative to a parental antibody lacking the masking moiety (MM), or the difference in affinity for a target antigen (such as CTLA4) of an activatable antibody comprising a masking moiety (MM) (before activation) relative to the affinity for the target antigen of the activatable antibody after activation). In some embodiments, the masking efficiency is measured by dividing the EC₅₀ for binding of an activatable antibody comprising a masking moiety (MM) (before activation) by the EC₅₀ of the parental antibody (e.g., by measuring EC₅₀ by ELISA; see e.g., the methods of Example 8). In some embodiments, masking efficiency is measured as the difference in affinity of an activatable antibody comprising the masking moiety (MM) for binding its target before activation relative to the affinity of the activatable antibody comprising the masking moiety (MM) for binding its target after activation (e.g., the difference in affinity for a target antigen (such as CTLA4) of an activatable antibody before activation relative to the activatable antibody after activation). In some embodiments, the masking moiety (MM) binds to the target binding moiety (TBM), and prevents the activatable antibody from binding to its target (e.g., an “inactive” activatable antibody). In some embodiments, the masking moiety (MM) has a dissociation constant for binding to the target binding moiety (TBM) that is greater than the dissociation constant of the target binding moiety (TBM) for its target.

In some embodiments, the masking moiety (MM) does not interfere with, obstruct, reduce the ability of, prevent, inhibit, or compete with the target binding moiety (TBM) for binding to its target after the activatable antibody has been activated (e.g., activated by treatment with one or more proteases that cleave within the cleavable moiety (CM), activated by a change in pH (increased or decreased), activated by a temperature shift (increased or decreased), activated after being contacted with a second molecule (such as an enzyme or a protein ligand), etc.). In some embodiments, the masking moiety (MM) does not interfere with, obstruct, reduce the ability of, prevent, inhibit, or compete with the target binding moiety (TBM) for binding its target after the cleavable moiety (CM) has been cleaved by one or more proteases that cleave within the cleavable moiety (CM). In some embodiments, the masking moiety (MM) has a masking efficiency of at most about 1.75 (e.g., at most about 1.75, at most about 1.5, at most about 1.4, at most about 1.3, at most about 1.2, at most about 1.1, at most about 1.0, at most about 0.9, at most about 0.8, at most about 0.7, at most about 0.6, or at most about 0.5, etc.) after activation (e.g., the relative affinity of the activatable antibody after activation as compared to the affinity of a parental antibody).

In some embodiments, an activatable antibody of the present disclosure: contains a masking moiety (MM) comprising a pair of cysteine residues at fixed positions to ensure that the activatable antibodies have constrained conformations, and/or harbor few or no chemically labile residues (such as methionine or tryptophan). Advantageously, the inclusion of a pair of cysteine residues at fixed positions ensured that the activatable antibodies had constrained conformations, tending to exhibit increased binding affinity and/or specificity. Furthermore, activatable antibodies of the present disclosure included masking moieties with few to no unfavorable residues for manufacturing processes, such as methionine or tryptophan.

### Mashing Moieties and Cleavable Linkers

In particular embodiments, the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221). In such embodiments, the MM and CM, from N-terminus to C-terminus comprises an amino acid sequence EVGSYPNPSSDCVPYYYACAY SGRSAGGGGTPLGLAGSGGS (SEQ ID NO:200). In some embodiments, the MM and the CM are covalently attached to the N-terminus of the light chain of the anti-CTLA4 antibody. In some embodiments, the MM and CM, from N-terminus to C-terminus comprises an amino acid sequence that has at least 90% or at least 95% sequence identity to SEQ ID NO:200.

In some embodiments, any of the masking moieties (MMs) described herein may further comprise one or more additional amino acid sequences (e.g., one or more polypeptide tags). Examples of suitable additional amino acid sequence may include, without limitation, purification tags (such as his-tags, flag-tags, maltose binding protein and glutathione-S-transferase tags), detection tags (such as tags that may be detected photometrically (e.g., red or green fluorescent protein, etc.)), tags that have a detectable enzymatic activity (e.g., alkaline phosphatase, etc.), tags containing secretory sequences, leader sequences, and/or stabilizing sequences, protease cleavage sites (e.g., furin cleavage sites, TEV cleavage sites, Thrombin cleavage sites), and the like. In some embodiments, the one or more additional amino acid sequences are at the N-terminus of the masking moiety (MM). In some embodiments, the additional amino acid sequence comprises or consists of the sequence EVGSY (SEQ ID NO: 148).

In some embodiments, the masking moiety binds to the target binding moiety (TBM) and inhibits the activatable antibody from binding to its target before activation (e.g., before treatment with one or more proteases that cleave within the cleavable moiety (CM), before undergoing a (local) change in pH (increased or decreased), before a temperature shift (increased or decreased), before being contacted with a second molecule (such as a small molecule or a protein ligand), etc.), but does not bind to the TBM and/or inhibit the activatable antibody from binding to its target after activation (e.g., after treatment with one or more proteases that cleave within the cleavable moiety (CM), after undergoing a (local) change in pH (increased or decreased), after a temperature shift (increased or decreased), after being contacted with a second molecule (such as a small molecule or a protein ligand), etc.). In some embodiments, the masking moiety (MM) inhibits binding of an activatable antibody to its target when the CM is not cleaved, but does not inhibit binding of the activatable antibody to its target when the CM is cleaved. In some embodiments, the masking moiety (MM) has a dissociation constant for binding to the TBM that is greater (e.g., at least about 1.5-fold greater, at least about 2-fold greater, at least about 2.5-fold greater, at least about 3-fold greater, at least about 3.5-fold greater, at least about 4-fold greater, at least about 4.5-fold greater, at least about 5-fold greater, at least about 10-fold greater, at least about 100-fold greater, at least about 500-fold greater, etc.) than the dissociation constant of the activatable antibody for its target (when in active form).

### Activatable Anti-CTLA4 Antibodies

In some embodiments, the activatable anti-CTLA4 antibody comprises a heavy chain comprising the amino acid sequence of SEQ ID NO:320 and a light chain comprising the amino acid sequence of SEQ ID NO:322. In some embodiments, the activatable anti-CTLA4 antibody comprises a heavy chain comprising the amino acid sequence of SEQ ID NO:321 and a light chain comprising the amino acid sequence of SEQ ID NO:322. The activatable antibody having heavy chain SEQ ID No: 320 and light chain SEQ ID No. 322 is referred to as TY22404. In some embodiments, the activatable antibody comprises a heavy chain comprising the amino acid sequence of SEQ ID NO:320 and a light chain comprising the amino acid sequence of SEQ ID NO: 322. In some embodiments, the activatable anti-CTLA4 antibody comprises a heavy chain having at least 90% (e.g., at least 92%, at least 93%, at least 94%, at least 95%, at least 96%, at least 97%, at least 98% or at least 99%) sequence identity to the amino acid sequence of SEQ ID NO: 320. In some embodiments, the activatable anti-CTLA4 antibody comprises a light chain having at least 90% (e.g., at least 92%, at least 93%, at least 94%, at least 95%, at least 96%, at least 97%, at least 98% or at least 99%) sequence identity to the amino acid sequence of SEQ ID NO: 321. In some embodiments, the activatable anti-CTLA4 antibody comprises a heavy chain having at least 90% (e.g., at least 92%, at least 93%, at least 94%, at least 95%, at least 96%, at least 97%, at least 98% or at least 99%) sequence identity to the amino acid sequence of SEQ ID NO: 320. In some embodiments, the activatable anti-CTLA4 antibody comprises a light chain having at least 90% (e.g., at least 92%, at least 93%, at least 94%, at least 95%, at least 96%, at least 97%, at least 98% or at least 99%) sequence identity to the amino acid sequence of SEQ ID NO: 322. In some embodiments, the activatable antibody is TY22404.

The activatable antibodies described herein may be further modified. In some embodiments, the activatable antibodies are linked to an additional molecular entity. Examples of additional molecular entities include pharmaceutical agents, peptides or proteins, detection agent or labels, and antibodies.

In some embodiments, an activatable antibody of the present disclosure is linked to a pharmaceutical agent. Examples of pharmaceutical agents include cytotoxic agents or other cancer therapeutic agents, and radioactive isotopes. Specific examples of cytotoxic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Examples of radioactive isotopes that can be conjugated to antibodies for use diagnostically or therapeutically include, but are not limited to, iodine¹³¹, indium¹¹¹, yttrium⁹⁰ and lutetium¹⁷⁷. Methods for linking a polypeptide to a pharmaceutical agent are known in the art, such as using various linker technologies. Examples of linker types include hydrazones, thioethers, esters, disulfides and peptide-containing linkers. For further discussion of linkers and methods for linking therapeutic agents to antibodies see e.g., Saito et al., Adv. Drug Deliv. Rev. 55:199-215 (2003); Trail, et al., Cancer Immunol. Immunother. 52:328-337 (2003); Payne, Cancer Cell 3:207-212 (2003); Allen, Nat. Rev. Cancer 2:750-763 (2002); Pastan and Kreitman, Curr. Opin. Investig. Drugs 3:1089-1091 (2002); Senter and Springer (2001) Adv. Drug Deliv. Rev. 53:247-264.

### V. PD-1 Antagonist

In one embodiment, the PD-1 antagonist useful in the treatment, medicaments and uses of the present invention include a monoclonal antibody (mAb), or antigen binding fragment thereof, that specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1, and useful in the treatment method, medicaments and uses of the present invention, are described in U.S. Pat. Nos. 7,488,802, 7,521,051, 8,008,449, 8,354,509, and 8,168,757, and International application publn. nos. WO2004/004771, WO2004/072286, WO2004/056875, US2011/0271358, and WO 2008/156712. Specific anti-human PD-1 mAbs useful as the PD-1 antagonist in the treatment method, medicaments and uses of the present invention include: pembrolizumab (also known as MK-3475), a humanized IgG4 mAb with the structure described in WHO Drug Information, Vol. 27, No. 2, pages 161-162 (2013) and that comprises the heavy and light chain amino acid sequences shown in Table B; nivolumab (BMS-936558), a human IgG4 mAb with the structure described in WHO Drug Information, Vol. 27, No. 1, pages 68-69 (2013); the humanized antibodies h409A11, h409A16 and h409A17, which are described in WO2008/156712, and AMP-514, which is being developed by MedImmune; cemiplimab; camrelizumab; sintilimab; tislelizumab; and toripalimab. Additional anti-PD-1 antibodies contemplated for use herein include MEDI0680 (U.S. Pat. No. 8,609,089), BGB-A317 (U.S. Patent publ. no. 2015/0079109), INCSHR1210 (SHR-1210) (PCT International application publ. no. WO2015/085847), REGN-2810 (PCT International application publ. no. WO2015/112800), PDR001 (PCT International application publ. no. WO2015/112900), TSR-042 (ANB011) (PCT International application publ. no. WO2014/179664) and STI-1110 (PCT International application publ. no. WO2014/194302).

Examples of mAbs that bind to human PD-L1, and useful in the treatment method, medicaments and uses of the present invention, are described in U.S. Pat. No. 8,383,796. Specific anti-human PD-L1 mAbs useful as the PD-1 antagonist in the treatment method, medicaments and uses of the present invention include BMS-936559, MEDI4736, and MSB0010718C.

In some embodiments, the PD-1 antagonist is pembrolizumab ((KEYTRUDA®, Merck Sharp & Dohme LLC, Rahway, NJ, USA), nivolumab (OPDIVO™, Bristol-Myers Squibb Company, Princeton, NJ, USA), atezolizumab (TECENTRIQ™, Genentech, San Francisco, CA, USA), durvalumab (IMFINZI™, AstraZeneca Pharmaceuticals LP, Wilmington, DE), cemiplimab (LIBTAYO™, Regeneron Pharmaceuticals, Tarrytown, NY, USA) avelumab (BAVENCIO™, Merck KGAA, Darmstadt, Germany) or dostarlimab (JEMPERLI™, GlaxoSmithKline LLC, Philadelphia, PA). In other embodiments, the PD-1 antagonist is pidilizumab (U.S. Pat. No. 7,332,582), AMP-514 (MedImmune LLC, Gaithersburg, MD, USA), PDR001 (U.S. Pat. No. 9,683,048), BGB-A317 (U.S. Pat. No. 8,735,553), or MGA012 (MacroGenics, Rockville, MD).

In one embodiment, the PD-1 antagonist useful in the methods of the invention is an anti-PD-1 antibody that blocks the binding of PD-1 to PD-L1 and PD-L2. In some embodiments of the treatment methods, medicaments and uses of the present invention, the PD-1 antagonist is a monoclonal antibody, or antigen binding fragment thereof, that comprises: (a) a light chain variable region comprising light chain CDR1, CDR2 and CDR3 of SEQ ID NOs: 10, 11 and 12, respectively and (b) a heavy chain variable region comprising heavy chain CDR1, CDR2 and CDR3 of SEQ ID NOs: 15, 16 and 17, respectively.

In other embodiments of the treatment methods, medicaments and uses of the present invention, the PD-1 antagonist is a monoclonal antibody, or antigen binding fragment thereof, that specifically binds to human PD-1 and comprises (a) a heavy chain variable region comprising SEQ ID NO:18 or a variant thereof, and (b) a light chain variable region comprising SEQ ID NO:13 or a variant thereof. A variant of a heavy chain variable region sequence is identical to the reference sequence except having up to six conservative amino acid substitutions in the framework region (i.e., outside of the CDRs). A variant of a light chain variable region sequence is identical to the reference sequence except having up to three conservative amino acid substitutions in the framework region (i.e., outside of the CDRs).

In another embodiment of the treatment methods, medicaments and uses of the present invention, the PD-1 antagonist is a monoclonal antibody that specifically binds to human PD-1 and comprises (a) a heavy chain comprising SEQ ID NO: 19 and (b) a light chain comprising SEQ ID NO:14. In one embodiment, the PD-1 antagonist is an anti-PD-1 antibody that comprises two heavy chains and two light chains, and wherein the heavy and light chains comprise the amino acid sequences in SEQ ID NO:19 and SEQ ID NO:14, respectively.

In all of the above treatment methods, medicaments and uses, the PD-1 antagonist inhibits the binding of PD-L1 to PD-1, and in specific embodiments also inhibits the binding of PD-L2 to PD-1. In some embodiments of the above treatment methods, medicaments and uses, the PD-1 antagonist is a monoclonal antibody, or an antigen binding fragment thereof, that specifically binds to PD-1 or to PD-L1 and blocks the binding of PD-L1 to PD-1.

Table B below provides a list of the amino acid sequences of exemplary anti-PD-1 mAbs for use in the treatment method, medicaments and uses of the present invention.

TABLE B
Exemplary PD-1 Antibody Sequences

| | | |
|---|---|---|
| Antibody | | SEQ |
| Feature | Amino Acid Sequence | ID NO. |

Pembrolizumab Light Chain

| | | |
|---|---|---|
| CDR1 | RASKGVSTSGYSYLH | 10 |
| CDR2 | LASYLES | 11 |
| CDR3 | QHSRDLPLT | 12 |
| Variable | EIVLTQSPATLSLSPGERATLSCRASKGVSTSGYSYLHWY | 13 |
| Region | QQKPGQAPRLLIYLASYLESGVPARFSGSGSGTDFTLTIS | |
| | SLEPEDFAVYYCQHSRDLPLTFGGGTKVEIK | |
| Light Chain | EIVLTQSPATLSLSPGERATLSCRASKGVSTSGYSYLHWYQ | 14 |
| | QKPGQAPRLLIYLASYLESGVPARFSGSGSGTDFTLTISSL | |
| | EPEDFAVYYCQHSRDLPLTFGGGTKVEIKRTVAAPSVFIFP | |
| | PSDEQLKSGTASVVCLLNNFYPREAKVQWKVDNALQSGN | |
| | SQESVTEQDSKDSTYSLSSTLTLSKADYEKHKVYACEVTH | |
| | QGLSSPVTKSFNRGEC | |

Pembrolizumab Heavy Chain

| | | |
|---|---|---|
| CDR1 | NYYMY | 15 |
| CDR2 | GINPSNGGTNFNEKFKN | 16 |
| CDR3 | RDYRFDMGFDY | 17 |
| Variable | QVQLVQSGVEVKKPGASVKVSCKASGYTFTNYYMYWVR | 18 |
| Region | QAPGQGLEWMGGINPSNGGTNFNEKFKNRVTLTTDSSTT | |
| | TAYMELKSLQFDDTAVYYCARRDYRFDMGFDYWGQGTT | |
| | VTVSS | |
| Heavy | QVQLVQSGVEVKKPGASVKVSCKASGYTFTNYYMYWVR | 19 |
| Chain | QAPGQGLEWMGGINPSNGGTNFNEKFKNRVTLTTDSSTT | |
| | TAYMELKSLQFDDTAVYYCARRDYRFDMGFDYWGQGTTV | |
| | TVSSASTKGPSVFPLAPCSRSTSESTAALGCLVKDYFPEP | |
| | VTVSWNSGALTSGVHTFPAVLQSSGLYSLSSVVTVPSSSL | |
| | GTKTYTCNVDHKPSNTKVDKRVESKYGPPCPPCPAPEFLG | |
| | GPSVFLFPPKPKDTLMISRTPEVTCVVVDVSQEDPEVQFN | |
| | WYVDGVEVHNAKTKPREEQFNSTYRVVSVLTVLHQDWLN | |
| | GKEYKCKVSNKGLPSSIEKTISKAKGQPREPQVYTLPPSQ | |
| | EEMTKNQVSLTCLVKGFYPSDIAVEWESNGQPENNYKTT | |
| | PPVLDSDGSFFLYSRLTVDKSRWQEGNVFSCSVMHEALH | |
| | NHYTQKSLSLSLGK | |

TABLE C

| Additional PD-1 Antibodies and Antigen | |
|---|---|
| Binding Fragments Useful in the | |
| Formulations, Methods and Uses of the Invention. | |
| A. Antibodies and antigen binding fragments | |
| comprising light and heavy chain | |
| CDRs of hPD-1.08A in WO2008/156712 | |
| CDRL1 | SEQ ID NO:20 |
| CDRL2 | SEQ ID NO:21 |
| CDRL3 | SEQ ID NO:22 |
| CDRH1 | SEQ ID NO:23 |
| CDRH2 | SEQ ID NO:24 |
| CDRH3 | SEQ ID NO:25 |
| C. Antibodies and antigen binding fragments | |
| comprising the mature h109A heavy | |
| chain variable region and one of the mature K09A | |
| light chain variable regions in | |
| WO 2008/156712 | |
| Heavy chain VR | SEQ ID NO:26 |
| Light chain VR | SEQ ID NO:27 8 |
| | or SEQ ID NO:2 |
| | or SEQ ID NO:29 |
| D. Antibodies and antigen binding | |
| fragments comprising the mature 409 heavy | |
| chain and one of the mature K09A | |
| light chains in WO 2008/156712 | |
| Heavy chain | SEQ ID NO:30 |
| Light chain | SEQ ID NO:31 |
| | or SEQ ID NO:32 |
| | or SEQ ID NO:33 |

In one embodiment, the anti-PD-1 antibody or antigen-binding fragment thereof comprises a heavy chain constant region, e.g. a human constant region, such as g1, g2, g3, or g4 human heavy chain constant region or a variant thereof. In another embodiment, the anti-PD-1 antibody or antigen-binding fragment thereof comprises a light chain constant region, e.g. a human light chain constant region, such as lambda or kappa human light chain region or a variant thereof. By way of example, and not limitation, the human heavy chain constant region can be g4 and the human light chain constant region can be kappa. In an alternative embodiment, the Fc region of the antibody is g4 with a Ser228Pro mutation (Schuurman, J et.al., Mol. Immunol. 38:1-8, 2001). In some embodiments, different constant domains may be appended to humanized VL and VH regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized. Although human IgG1 antibodies provide for long half-life and for effector functions, such as complement activation and antibody-dependent cellular cytotoxicity, such activities may not be desirable for all uses of the antibody. In such instances a human IgG4 constant domain, for example, may be used. The present invention includes the use of anti-PD-1 antibodies or antigen-binding fragments thereof which comprise an IgG4 constant domain. In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, in order to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Imunol. 30:105. In other instances, a modified IgG1 constant domain which has been modified to increase half-life or reduce effector function can be used.

In another embodiment, the PD-1 antagonist is an antibody or antigen binding protein that has a variable light domain and/or a variable heavy domain with at least 95%, 90%, 85%, 80%, 75% or 50% sequence identity to one of the variable light domains or variable heavy domains described above, and exhibits specific binding to PD-1. In another embodiment of the methods of treatment of the invention, the PD-1 antagonist is an antibody or antigen binding protein comprising variable light and variable heavy domains having up to 1, 2, 3, 4, or 5 or more amino acid substitutions, and exhibits specific binding to PD-1.

In some embodiments, pembrolizumab is administered at a dose of about 400 mg every 6 weeks.

In some embodiment, pembrolizumab is administered at a dose of about 2 mg/kg. In some embodiment, pembrolizumab is administered at a dose of about 2 mg/kg every three weeks. In particular embodiments, the patient is a pediatric patient.

In some embodiment, pembrolizumab is administered as a 30 minute (−5 minutes/+10 minutes) intravenous infusion. In one embodiment, the selected dose of pembrolizumab is administered by IV infusion over a time period of between 25 and 40 minutes, or about 30 minutes.

In one aspect, pembrolizumab in included in a pharmaceutical composition with a pharmaceutically acceptable carrier or diluent and may include additional pharmaceutically acceptable excipients.

### VI. Pharmaceutical Compositions, Kits, and Articles of Manufacture

The activatable anti-CTLA4 antibodies described herein and the pembrolizumab can be administered in a pharmaceutical compositions comprising a pharmaceutically acceptable carrier. The activatable anti-CTLA4 antibody and the pembrolizumab can be administered in separate pharmaceutical compositions or in a single pharmaceutical composition. The compositions can be prepared by conventional methods known in the art.

The term “pharmaceutically acceptable carrier” refers to any inactive substance that is suitable for use in a formulation for the delivery of an active agent (e.g., the activatable anti-CTLA4 antibody or pembrolizumab). A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride. The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering an activatable anti-CTLA4 antibody is a sterile liquid, such as a solution, suspension, or dispersion, for injection or infusion. Sterile solutions can be prepared by incorporating the antibody in the required amount in an appropriate carrier, followed by sterilization microfiltration. Generally, dispersions are prepared by incorporating the antibody into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

In some embodiments, there is provided an article of manufacture comprising materials useful for the treatment of a cancer. The article of manufacture can comprise a container and a label or package insert on or associated with the container. Suitable containers include, for example, bottles, vials, syringes, etc. The containers may be formed from a variety of materials such as glass or plastic. Generally, the container holds a composition, which is effective for treating a cancer, described herein, and may have a sterile access port (for example, the container may be an intravenous solution bag or a vial having a stopper pierceable by a hypodermic injection needle). Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a cancer. The label or package insert may further comprise instructions for administering the composition to a patient.

Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a cancer described herein, optionally in combination with the articles of manufacture. Kits of the present application include one or more containers comprising any one of the compositions described herein (or unit dosage form and/or article of manufacture). In some embodiments, the kit further comprises other agents (e.g., one or more additional therapeutic agents) and/or instructions for use in accordance with any of the methods described herein. The kit may further comprise a description of selection of individuals suitable for treatment. Instructions supplied in the kits of the present application are typically written instructions on a label or package insert (e.g., a paper sheet included in the kit), but machine-readable instructions (e.g., instructions carried on a magnetic or optical storage disk) are also acceptable.

For example, in some embodiments, there is provided a kit comprising a pharmaceutical composition comprising any one of the activatable anti-CTLA4 antibodies described herein and a pharmaceutically acceptable carrier; pembrolizumab and a pharmaceutically acceptable carrier; and instructions for administering the pharmaceutical composition to a subject (e.g., a human patient) having a cancer. In some embodiments, the kit further comprises a pharmaceutical composition comprising an additional therapeutic agent, such as a chemotherapeutic agent. In some embodiments, the kit comprises one or more assays or reagents thereof for determining a level of one or more biomarkers described herein (e.g., CD8+ T cells, CD4+ T cells, CD8+ Tₑₘ cells, CD4+ Tₑₘ cells, T_{reg} cells, a ratio of CD8+ Tₑₘ cells to T_{reg} cells, a ratio of CD4+ Tₑₘ cells to T_{reg} cells, NK cells, B cells).

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The containers may be unit doses, bulk packages (e.g., multi-dose packages) or sub-unit doses. Kits may also include multiple unit doses of the pharmaceutical compositions and instructions for use and packaged in quantities sufficient for storage and use in pharmacies, for example, hospital pharmacies and compounding pharmacies.

### EXAMPLES

The invention can be further understood by reference to the following examples, which are provided by way of illustration and are not meant to be limiting.

### Example 1. a Phase 1b/2, Open-Label, Dose Escalation and Expansion Study of TY22404 in Combination with Pembrolizumab (Anti PD-1 Antibody) in Patients with Advanced/Metastatic Solid Tumors

In a Phase 1b/2 study, TY22404 monotherapy demonstrated an unprecedented safety profile (No G3 or higher TRAEs) up to 20 mg/kg Q3W with repeat dosing and clinical activity in heavily pre-treated patients. The following example describes the preliminary results from dose escalation of TY22404 in combination with pembrolizumab in patients of advanced/metastatic solid tumors including the cervical, colorectal, endometrial, neuroendocrine, ovarian and pancreatic carcinomas (NCT05405595). TY22404 wasadministered IV over a period of 60-90 minutes. Pembrolizumab (KEYTRUDA®, Merck Sharp & Dohme LLC, Rahway, NJ, USA) is administered IV over a period of 30 minutes. For the TY22404-pembrolizumab combination regimen, the starting dose of TY22404 was administered 30-60 minutes after the end of pembrolizumab infusion.

Objectives. Primary objectives of the study are to assess the safety and tolerability of TY22404 at escalating dose levels, in combination with pembrolizumab in adults with advanced/metastatic solid tumors at escalating dose levels; and to determine the maximum tolerated dose (MTD) and recommended Phase 2 dose (RP2D) for TY22404 in combination with pembrolizumab; and to assess the preliminary antitumor activity of TY22404-pembrolizumab combination regimen in dose expansion. The secondary objectives of the study are to assess the pharmacokinetic (PK) profile of TY22404 and pembrolizumab; to assess dose proportionality of key PK parameters (area under the time concentration curve [AUC], maximum concentration [Cmax] to assess the immunogenicity of TY22404 and pembrolizumab; to characterize the relationship between immunogenicity (anti-drug antibody [ADA] positivity) and PK, safety, and efficacy parameters; to evaluate the preliminary antitumor activity of TY22404-pembrolizumab combination regimens in dose expansion. To assess the safety and tolerability of TY22404 in combination with pembrolizumab in adults with advanced/metastatic solid tumors; to assess the PK profile of TY22404 and pembrolizumab; to assess the immunogenicity of TY22404 and pembrolizumab in dose expansion. And the exploratory objectives to assess pharmacodynamic and predictive biomarkers, including but not limited to serum proteins such as cytokines, etc., peripheral immune cell subset profiling, tumor infiltrated lymphocytes, and pharmacogenomics markers, and cleavage of TY22404 in post-treatment tumor tissues (if available).

Study Design. This is a Phase 1b/2, open label, multicenter, dose-escalation and dose expansion study to evaluate the safety, tolerability, PK, and preliminary efficacy of an TY22404-pembrolizumab combination regimen in patients with advanced/metastatic solid tumors. After a screening period of up to 28 days, qualified patients were enrolled to receive their assigned dose regimen of TY22404-pembrolizumab combination regimen. TY22404 and pembrolizumab was dosed Q3W/Q6W or Q3W, respectively, until progressive disease (PD), intolerable toxicities, withdrawal of consent, or up to 35 cycles (Q3W). An IV infusion of TY22404 over 60-90 minutes was administered 30-60 minutes after the administration of pembrolizumab.

A treatment cycle consists of 21 days. Patients treated with TY22404 and pembrolizumab on Day 1 of each treatment cycle while on study until documented confirmed progressive disease (PD) according to RECIST v1.1 and/or iRECIST, development of significant toxicity, withdrawal of consent, or other discontinuation/withdrawal reason, or up to 35 cycles, whichever occurs first. An intravenous (IV) infusion of TY22404 over 60-90 minutes was administered 30-60 minutes after the end of the pembrolizumab infusion. Pembrolizumab was administered following its approved Prescription Information.

Patients who discontinue treatment due to intolerable AEs related to TY22404-pembrolizumab combination regimen was followed until the AEs have returned to Grade 0 or 1, or become stable, or until the patient receives new non-protocol treatment. During the study, patients were evaluated for safety and toxicity, PK, immunogenicity, objective response, DCR, DOR, PFS, OS, and biomarkers.

### Dose Escalation Phase

A modified Toxicity Probability Interval (mTPI) design with a target DLT rate of approximately 20% and the equivalence interval (EI) of [0.15, 0.23], in which any dose is considered as a potential candidate for the true MTD, was applied for dose escalation and confirmation to determine RP2D for TY22404 in combination with pembrolizumab. Dose levels are shown in Table 1. For TY22404 combination treatment with pembrolizumab, both drugs were dosed Q3W and/or Q6W for TY22404 with pembrolizumab remaining constant at 200 mg Q3W for up to 35 cycles for both dose levels (DL1 and 2) of TY22404.

TABLE 1
TY22404-Pembrolizumab Dose Levels

| | Dose Level | TY22404 | Pembrolizumab |
|---|---|---|---|
| Design | (DL) | (mg/kg, Q3W and/or Q6W) | (mg, Q3W) |
| mTPI design | DL1 | ≤6 | 200 |
| | DL2 | ≤10 | 200 |

DL = dose level; mTPI = modified Toxicity Probability Interval; Q3W = every 3 weeks; Q6W = every 6 weeks.

The dose escalation started at 6 mg/kg Q3W following the mTPI design (DL1). Should this be tolerated, dosing proceeded to 10 mg/kg Q3W. If 6 mg/kg Q3W was not tolerated based on early or late toxicities per SRC review, then dosing proceeded at 6 mg/kg Q6W. If 6 mg/kg Q6W was tolerated, based on SRC review of early and late toxicities, then dosing proceeded at 10 mg/kg Q6W. Similarly, if 6 mg/kg Q3W was tolerated, but 10 mg/kg Q3W was not tolerated, based on SRC review of early and late toxicities, then dosing proceeded at 10 mg/kg Q6W.

DLTs were assessed using the National Cancer Institute Common Terminology Criteria for Adverse Events (NCI CTCAE) v5.0. The safety and tolerability of each TY22404 dose level was assessed by SRC after all patients enrolled in the dose level have been followed for at least 21 days after the first dose of TY22404-pembrolizumab combination regimen (DLT observation period). The dose and dosing frequency of pembrolizumab will not be changed.

The active dose (or range) was defined based on safety, PK/pharmacodynamic data and its modeling to predict anti-tumor response, and early signs of efficacy during dose escalation, and was further evaluated in dose expansion. After completing the dose expansion, RP2D, which include the MTD or MAD, dose levels below the MTD or MAD, as well as intermediate doses between the prespecified dose levels, were determined based on an overall assessment of all safety data, all available PK and pharmacodynamic data, and documented objective response observations during both dose escalation and dose expansion. The RP2D is a confirmed optimal pharmacologically active dose. In addition, RP2D may include loading dose(s) followed by maintenance dose at reduced dose levels and/or reduced dosing frequency (or frequencies), pending emerging data from combination DL1 and DL2.

### Dose Expansion

After the active dose (or range) of TY22404-pembrolizumab combination regimen are determined from dose escalation, up to 60 patients with up to 20 each of microsatellite stable colorectal cancer (MSS CRC) patients, head and neck squamous cell carcinoma (HNSCC) patients who have not received a prior immunotherapy and HNSCC patients who have received a prior anti-programmed death protein 1 (anti-PD-1) therapy was treated at the active dose (or range of doses) to further evaluate the anti-tumor activities of the combination regimen. For each of the 3 tumor types, the first 10 patients were treated with the one dose/schedule (such as 6 mg/kg TY22404 Q3W or Q6W) and the next 10 patients were treated with the 2nd dose/schedule, which could include a higher continuous repeat dose (such as 10 mg/kg TY22404 Q3W or Q6W) or a higher loading dose (such as 10 mg/kg TY22404) followed by a lower maintenance dose Q3W or Q6W. The higher dose was identified primarily based on the PK and safety data from the continued combination dose escalation phase. For each indication, Simon two-stage design as outlined in Table 2 with patients from 2 different dose/scheduling combined was used and the final decision was made by the SRC based on the totality of the safety and efficacy information. Note: if the combination dose escalation phase has not identified an active dose or active doses with a tolerable Q3W interval, then all 20 patients per tumor type were treated at Q6W.

TABLE 2
Simon Two-Stage Design for Dose Expansion

| | ORR | | Stage 1 | | Overall Study | | | |
|---|---|---|---|---|---|---|---|---|
| | p₀ | p₁ | Sample | ≥# of | Sample | ≥# of | Type 1 | |
| Indication | (H₀: p ≤ p₀) | (H₀: p ≤ p₁) | Size | Responders | Size | Responders | Error | Power |
| HNSCC I/O | 20% | 35% | 12 | 2 | 20 | 6 | 0.194 | 75.1% |
| naïve | | | | | | | | |
| HNSCC I/O | 3% | 15% | 9 | 1 | 20 | 2 | 0.088 | 70.7% |
| experienced | | | | | | | | |
| MSS CRC | 10% | 35% | 9 | 2 | 20 | 4 | 0.091 | 86.3% |

HNSCC = head and neck squamous cell carcinoma; IO = immuno-oncology; MSS CRC = microsatellite stable colorectal cancer.
Stage 1 and the overall study are considered successful if at least the corresponding number of responders are observed, respectively.

The treatment proceeded beyond initial radiographic PD by RECIST v1.1 (i.e., unconfirmed radiographic progression) if the Investigator deems that it is in the patient's best interest, the patient provides a written consent or verbal consent to continue receiving trial treatment, and if the following criteria are met: Absence of clinical symptoms or signs indicating clinically significant disease progression; No decline in performance status; Absence of rapid disease progression or threat to vital organs or critical anatomical sites (e.g., central nervous system [CNS] metastasis, respiratory failure due to tumor compression, spinal cord compression) requiring urgent alternative medical intervention; No significant unacceptable or irreversible toxicities related to the trial treatment; No other treatment discontinuation criteria are met.

PD is confirmed as per iRECIST; a repeat scan was required, preferably in 4 weeks and no later than 8 weeks. Once PD is confirmed as per iRECIST, ongoing treatment was not allowed. Otherwise, the study treatments may continue for a total duration of up to 35 cycles, or until PD, unacceptable toxicity, or withdrawal of consent, whichever occurs first. Patients who discontinue treatment due to intolerable AEs related to TY22404-pembrolizumab combination regimen was followed until the AEs have resolved to Grade 0 or 1, or become stable, or until the patient receives new non protocol treatment. Inclusion Criteria

Patients who met all of the following inclusion criteria to be eligible for participation in this study:
1. ≥18 years of age at the time of informed consent.
2. Eastern Cooperative Oncology Group (ECOG) performance status 0 or 1 with no deterioration over the previous 2 weeks.
3. For Dose Escalation Phase Only: Patients with advanced or metastatic solid tumors, histologically or pathologically confirmed, who have progressed after all standard therapies, or for whom no further standard therapy exists.
4. Dose Expansion Phase Only: Patients must have one of the following tumor types including the corresponding criteria.

### MSS CRC

Advanced CRC not amenable to curative surgery, with MSS status per local or central laboratory assessment
Has received at least 2 and no more than 3 prior systemic treatments regimens
Free of liver metastasis
No prior immunotherapy
   HNSCC Who have Previously been Treated with an Immunotherapy Agent
Advanced HNSCC not amenable to curative surgery or radiation
No prior treatment with anti-cytotoxic T-lymphocyte antigen (anti-CTLA4) therapy
Had 1 or 2 prior treatment regimens, one of which must have included a PD-1 therapy
For the PD-1 included regimen, relapse or secondary resistance, but not primary resistance, must have been demonstrated:
   Anti-PD-1/L1 treatment secondary resistance is defined by meeting all of the following criteria:
a. Received at least 2 doses of an approved anti-PD-1 monoclonal antibody (mAb)
b. No documented PD within 3 months from the start of prior anti-PD-1 treatment. HNSCC patients who have PD within the first 3 cycles of a PD1/L1 therapy (regardless of whether the progression is confirmed later) are considered to have primary resistance and are not eligible for this protocol.
c. Demonstrated PD after PD-1 as defined by RECIST v1.1. The initial evidence of PD is to be confirmed by a second assessment no less than 4 weeks from the date of the first documented PD according to iRECIST, in the absence of rapid clinical progression. This determination is made by the Investigator. Once PD is confirmed, the initial date of PD documentation was considered the date of PD.
Patients having been treated with anti-cytotoxic T-lymphocyte antigen (anti-CTLA4) therapy are not eligible.
   HNSCC Who have not Previously been Treated with an Immunotherapy Agent
Advanced HNSCC not amenable to curative surgery or radiation
Must not have received a prior immunotherapy agent, and only 1 prior line of systemic chemotherapy is allowed.
The tumor PD-L1 combined positive score (CPS) based on fresh or archival tumor must be ≥1 using PD-L1 IHC 22C3 testing.
5. Patients should have at least 1 measurable lesion at baseline according to the definition of RECIST v1.1. Lesions situated in a previously irradiated area are considered measurable if progression has been demonstrated in such lesions.
6. Adequate hematologic function, defined by the following:
a. Absolute neutrophil count (ANC) ≥1.5×109/L, without the use of granulocyte colony stimulating factor (G-CSF) such as filgrastim within 2 weeks prior to study treatment.
b. Platelet count ≥75×109/L without transfusion within 2 weeks (≤14 days) prior to study treatment.
c. Hemoglobin ≥9 g/dL without transfusion or erythropoietin within 2 weeks (≤14 days) prior to study treatment.
7. Aspartate aminotransferase (AST) and alanine aminotransferase (ALT)≤2.5×upper limit of normal (ULN), and total bilirubin≤1.5×ULN. Exception: Patients who have serum bilirubin increases due to documented underlying Gilbert's Syndrome or familial benign unconjugated hyperbilirubinemia may be enrolled.
8. Adequate renal function defined by a creatinine clearance ≥45 mL/min (by Cockcroft-Gault formula).
9. Coagulation tests, defined by the following:
a. Activated partial thromboplastin time (aPTT)≤1.5×ULN.
b. International normalized ratio (INR)≤1.5×ULN. Exception: INR≤3×ULN is acceptable for patients on warfarin anticoagulation.
10. Washout period from previous antitumor therapies:
a. Small molecule inhibitors/chemotherapeutic drugs: at least 2 weeks or 5 half-lives, whichever is longer (6 weeks for nitrosoureas or mitomycin) before the first dose of study drug.
b. Large molecules including mAbs, bispecific antibodies, antibody-drug conjugates, fusion proteins: at least 4 weeks before the first dose of study drug
c. Autologous stem cell transplant (ASCT) or chimeric antigen receptor T cell (CAR-T), chimeric antigen receptor natural killer (CAR-NK) cell therapy: at least 3 months before the first dose of study drug
d. Radiotherapy for bone metastases or other nontarget lesions: at least 2 weeks before the first dose of study drug. Participants must have recovered from all radiation-related toxicities, not require corticosteroids, and not have had radiation pneumonitis.
   The following are the exceptions:
a. Hormonal therapy with gonadotropin-releasing hormone agonists or antagonists for prostate cancer
b. Hormone replacement therapy or oral contraceptives
c. Current or prior administration of denosumab (Xgeva), IV bisphosphonate, or oral bisphosphonate for preventing bone metastases-related complications.
11. Previous AEs have been improved to baseline or Grade≤1 NCI CTCAE v5.0 (except for patients with alopecia). Participants with Grade≤2 neuropathy may be eligible. Participants with endocrine-related AEs Grade≤2 requiring treatment or hormone replacement may be eligible.

Safety Evaluations. Safety assessments were carried out during specified periodic PEs, vital signs, ECOG performance status, laboratory variables (e.g., liver tests/monitoring, hematology, coagulation test, serum chemistry, urine test, and pregnancy test), ECG, and AEs. AEs are graded according to the NCI CTCAE v5.0.

Efficacy Evaluations. Tumor assessments for response/progression was performed at baseline, and every 6 weeks (±1 week) for the first 4 cycles. If treatment continues beyond 4 cycles, then assessments were carried out every 9 weeks (±1 week) for the remaining treatment duration thereafter, until PD or death, treatment/study discontinuation due to treatment toxicity, loss to follow-up, withdrawal of consent, start of new cancer treatment, or study completion/closure, whichever occursedfirst. Response and progression was evaluated in this study using the international criteria proposed by the revised RECIST v1.1 guideline(s), and/or iRECIST.

Pharmacokinetic and Immunogenicity Evaluations. Blood samples were collected from all patients determine the serum concentration of TY22404 and pembrolizumab. PK parameters for TY22404 was monitored more intensively during the first treatment cycle. Reduced PK sampling will be conducted for pembrolizumab. Non-compartmental analysis will be conducted using Phoenix WinNonlin Version 8.3 or above versions. PK parameters include, but were not limited to AUC0-21d, AUClast, AUCinf, Cmax, Tmax, t1/2, MRT, CL, Vss will be reported. Dose proportionality will also be assessed for AUC and Cmax. Blood samples for ADAs against TY22404 will be collected at predose of cycles 1-4, and every 4 cycles thereafter if treatment will continue beyond 4 cycles. Reduced ADA sampling will be conducted for pembrolizumab. Additionally, ADA samples were collected at the end of treatment (EOT) and at 30 days when feasible after the last dose.

Pharmacodynamics Evaluations. Pharmacodynamic biomarkers for TY22404 was listed and summarized by protocol specified time point and treatment and included but are not limited to serum proteins such as cytokines, etc., peripheral immune cell subset profiling, tumor infiltrated lymphocytes and pharmacogenomics markers in tumor tissues (if available).

Tumor Evaluations. Tumor PD-L1 IHC 22C3 testing for CPS assessment was performed for all the patients in the expansion cohort. For the HNSCC patients who have not previously been treated with an immunotherapy agent and only such patients with CPS ≥1 were enrolled into the dose expansion phase study. Tumor resection/biopsy formalin-fixed paraffin-embedded (FFPE) samples (block or 10 unstained FFPE slides) within 2 years from C1D1 are required for the combination dose expansion cohorts. If no archived tumor sample available, tumor biopsy was collected at screening. The index/target lesions or radiated lesions were not be used for the biopsy. For CRC patients who did not have existing MSI status report, tumor samples (archived or fresh) was collected at screening for MSI testing either locally or in a central laboratory, and only MSS CRC patients were enrolled into the expansion cohort. Patients with biopsy accessible tumors may also undergo optional pre- and post-treatment tumor biopsies at baseline and during the 3rd week of Cycle 2 and/or at the EOT, respectively. Cleavage of TY22404 in the post-treatment fresh biopsy (if available) was be explored in additional to other relevant biomarkers. Patients were given a separate, specific written consent to provide baseline, on-treatment, and/or EOT biopsies.

### Interim Results

11 patients (Pts) had been treated with TY22404 (6 mg/kg Q3W and 10 mg/kg Q3W or Q6W)+Pembrolizumab (200 mg Q3W) in dose escalation. Patients were generally heavily pre-treated (Table 1). Tumor types consist of the ovarian, colorectal, pancreatic and endometrial cancer, etc., and most of them (82%) are generally considered as “cold” tumors.

TABLE 3
Baseline Characteristics of Patients

| Characteristics | N = 11 |
|---|---|
| Age (years), median (range) | 61 (26-75) |
| Female, n (%) | 9 (82%) |

Race, n (%)

| | |
|---|---|
| Caucasian, n (%) | 7 (64%) |
| Black or African American, n (%) | 1 (9%) |
| Other | 3 (27%) |

ECOG, n (%)

| | |
|---|---|
| 0 | 6 (55%) |
| 1 | 5 (45%) |

Prior lines of therapy before enrollment, n (%)

| | |
|---|---|
| ≥3 | 9 (82%) |
| Prior immunotherapy, n (%) | 2 (18%) |

### Clinical Safety Assessments

As shown in Table 4 and Table 5, no dose-limiting toxicities was observed at the TY22404 (6 mg/kg Q3W and 10 mg/kg Q3W or Q6W)+pembrolizumab (200 mg Q3W) in dose escalation. The most frequent TRAEs were fatigue (3 patients), diarrhea (2 patients), nausea (2 patients) and vomiting (2 patients). Most TRAEs are Grade (G) G1 and G2. There are two patients had G3 TRAEs: one G3 diarrhea as late onset toxicity (C8 in 6 mg/kg Q3W cohort) and one G3 adrenal insufficiency after DLT (C3 in 10 mg/kg Q6W cohort). No G4/5 events was observed, the early safety profile is comparable to that of pembrolizumab monotherapy. (Table 4 and Table 5).

TABLE 4
Frequencies of TRAEs with different Grades

| | Cohort | G1 | G2 | G3 | |
|---|---|---|---|---|---|
| TRAE by Cohort | N | (%) | (%) | (%) | G4/5 |
| TY22404 6 mg/kg Q3W | 5 | 0 | 2 | 1 | 0 |
| | | | (40%) | (20%) | |
| TY22404 10 mg/kg Q3W | 6 | 3 | 2 | 1 | 0 |
| or Q6W | | (50%) | (33%) | (17%) | |

TABLE 5
TRAEs and frequency for the dose escalation phase (N = 11).

| TRAEs | Grade 1 | Grade 2 | Grade 3 | Grade 4 |
|---|---|---|---|---|
| Fatigue | 2 (18.18%) | 1 (9.09%) | 0 | 0 |
| Hyperthyroidism | 2 (18.18%) | 0 | 0 | 0 |
| Diarrhea | 1 (9.09%) | 0 | 1 (9.09%) | 0 |
| Nausea | 1 (9.09%) | 1 (9.09%) | 0 | 0 |
| Vomiting | 1 (9.09%) | 1 (9.09%) | 0 | 0 |
| Stomatitis | 1 (9.09%) | 0 | 0 | 0 |
| Pruritus | 1 (9.09%) | 0 | 0 | 0 |
| Ear Pruritus | 1 (9.09%) | 0 | 0 | 0 |
| Cough | 1 (9.09%) | 0 | 0 | 0 |
| Fever | 1 (9.09%) | 0 | 0 | 0 |
| Joint stiffness | 1 (9.09%) | 0 | 0 | 0 |
| Arthralgia | 1 (9.09%) | 0 | 0 | 0 |
| Migratory Sensory Pain | 1 (9.09%) | 0 | 0 | 0 |
| Adrenal insufficiency | 0 | 0 | 1 (9.09%) | 0 |
| Anorexia | 0 | 1 (9.09%) | 0 | 0 |
| Bell's palsy | 0 | 1 (9.09%) | 0 | 0 |
| Musculoskeletal | 0 | 1 (9.09%) | 0 | 0 |
| stiffness | | | | |

### Clinical Activity Assessments

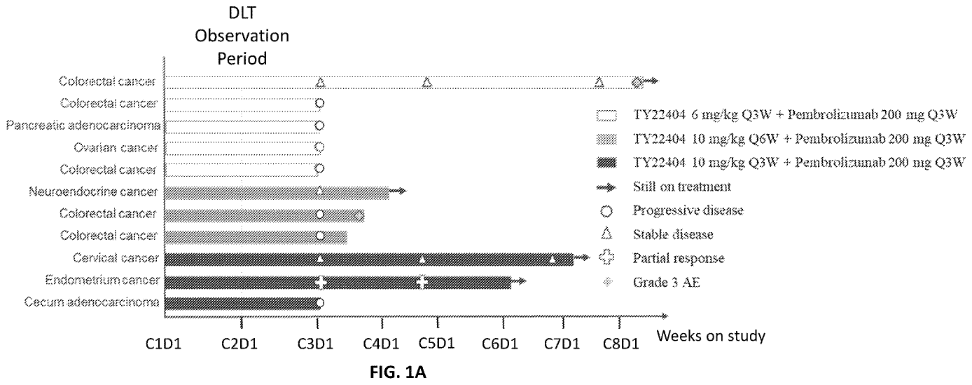

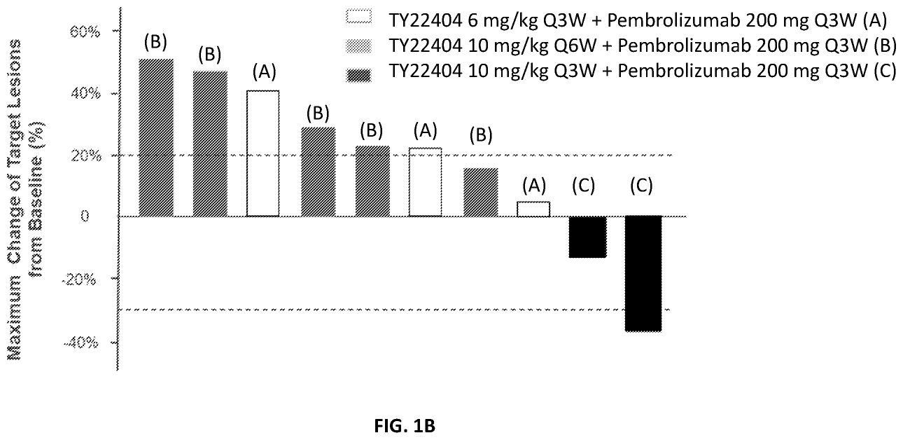

As shown in FIG. 1A, partial response (PR) has been observed in a patient who received TY22404 10 mg/kg Q3W+pembrolizumab 200 mg Q3W (see Case Study #1). The objective response rate (ORR) is 9%, and disease control rate (DCR) is 36% among 11 patients who received TY22404 (6 mg/kg Q3W, 10 mg/kg Q3W or Q6W)+pembrolizumab (200 mg Q3W) and had valid post-baseline tumor. Among the 6 patients who received TY22404 10 mg/kg Q3W/6W+pembrolizumab 200 mg Q3W, ORR is 17% and DCR is 50%. As shown in FIG. 1B, ne patient (10 mg/kg, Q3W; PD due to new lesion) did not have complete post-treatment target lesion measurement and is not.

### Clinical Case Studies

Case Study #1: As shown in Table 6, confirmed PR with 33% and 37% target lesion reduction at the end of C2 and C4 was observed in patient with Advanced adenocarcinoma of endometrium (MSI-H) with lung metastasis who received TY22404 10 mg/kg Q3W+pembrolizumab 200 mg Q3W. This patient previously received carboplatin+paclitaxel×6 cycles followed with anastrozole as a maintenance therapy until new lung metastasis lesion developed.

TABLE 6
Tumor assessments in a patient with
Metastatic Endometrial Cancer

| | Lesion | | | End of | End of |
|---|---|---|---|---|---|
| | # | Location | Baseline | C2 | C4 |
| Target | TL#1 | Lung, | 27 mm | 18 mm | 17 mm |
| lesion | | Right | | | |
| | Sum | | 27 mm | 18 mm | 17 mm |
| Non-target | | N/A | N/A | N/A | N/A |
| lesion | | | | | |
| New lesion | | | | No | No |
| Overall | | | | PR | PR |
| Response | | | | (−33%) | (−37%) |

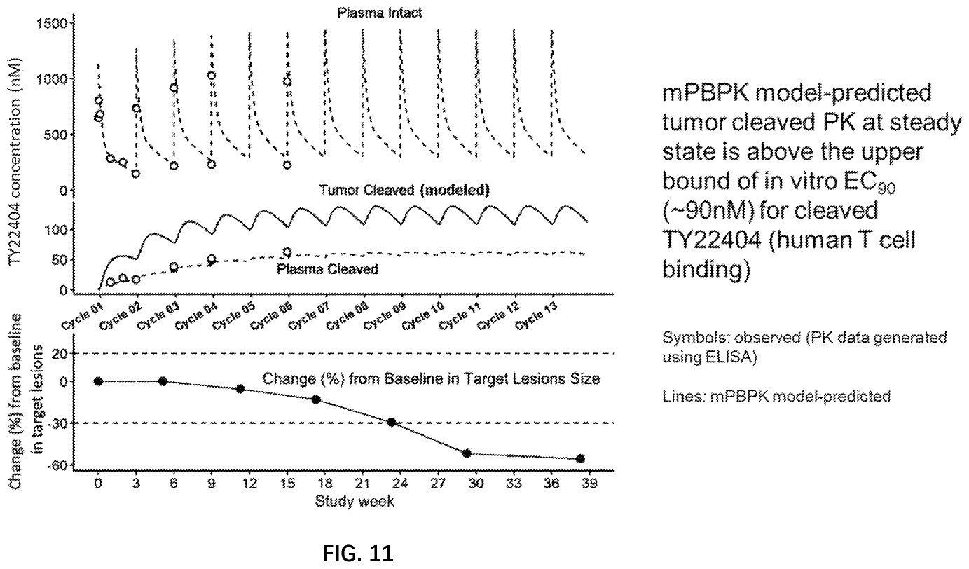

Case Study #2: As shown in Table 7, confirmed SD with 13% reduction in target lesions at the end of C6 was observed in patient Advanced cervical cancer (stage IV squamous carcinoma) with mediastinal lymph node metastasis who received TY22404 10 mg/kg Q3W+pembrolizumab 200 mg Q3W. The PD-L1 CPS score of this patient is 1, the TMB high is 24 Muts/Mb. This patient previously received 2 lines of therapies: carboplatin/paclitaxel/bevacizumab×6 cycles, pembrolizumab monotherapy×9 cycles. Supported by PK modeling, TY22404 10 mg/kg Q3W+Pembrolizumab shows ability to overcome Pembrolizumab resistance in a 3L cervical patient (FIG. 11).

TABLE 7
Tumor assessments in a patient with advanced
cervical cancer (stage IV squamous carcinoma)

| | Lesion | | | | | |
|---|---|---|---|---|---|---|
| | # | Location | Baseline | End of C2 | End of C4 | End of C6 |
| Target | TL#1 | Lymph node | 25 mm | 25 mm | 21 mm | 17 mm |
| lesion | | (Subcarinal) | | | | |
| | TL#2 | Lymph node | 29 mm | 29 mm | 30 mm | 30 mm |
| | | (Pre-carinal) | | | | |
| | Sum | | 54 mm | 54 mm | 51 mm | 47 mm |
| Non target | | Lymph node | Present | Present | Present | Present |
| lesion | | Right Supra- | | | | |
| | | clavicular | | | | |
| New lesion | | | | No | No | No |
| Overall | | | | SD (+0%) | SD (−5.6%) | SD (−13%) |
| Response | | | | | | |

As shown in Table 8, two partial responses (PRs) (2/11) have been observed in patient who received TY22404 (6 mg/kg Q3W, 10 mg/kg Q3W or Q6W)+pembrolizumab (200 mg Q3W) in the dose escalation stage, one of which is a cervical cancer patient progressed on pembrolizumab. Two initial PRs were observed in the MSS CRC expansion cohort, and 1 initial PR have been observed in the HNSCC expansion cohort in the dose expansion stage received TY22404 (10 mg/kg Q3W or Q6W)+pembrolizumab (200 mg Q3W).

TABLE 8
Tumor Assessments in Patients Doses with TY22404/Pembrolizumab

| | TY22404 + pembrolizumab | | | | | |
|---|---|---|---|---|---|---|
| TY22404 | Dose escalation | | | Dose expansion | | |
| Dosing | Patients Dosed | Safety | | Patients Dosed | Safety | |
| Regimen | (N) | (TRAEs) | Efficacy | (N) | (TRAEs) | Efficacy |
| 6 mg/kg Q3W | 5 | 20% G3 | ORR = | / | / | / |
| | (5 evaluable | | 0% | | | |
| | for efficacy) | | DCR = | | | |
| | | | 20% | | | |
| 10 mg/kg Q6W | 3 | 33% G3 | ORR = | 14 | 0% G3 | ORR = 8% |
| | (3 evaluable | | 0% | (13 evaluable | | DCR = 46% |
| | for efficacy) | | DCR = | for efficacy) | | |
| | | | 0% | | | |
| 10 mg/kg Q3W | 3 | 0% G3 | ORR = | 21 | 10% G3 | ORR = 15% |
| | (3 evaluable | | 67% | (13 evaluable | | DCR = 77% |
| | for efficacy) | | DCR = | for efficacy) | | |
| | | | 67% | | | |

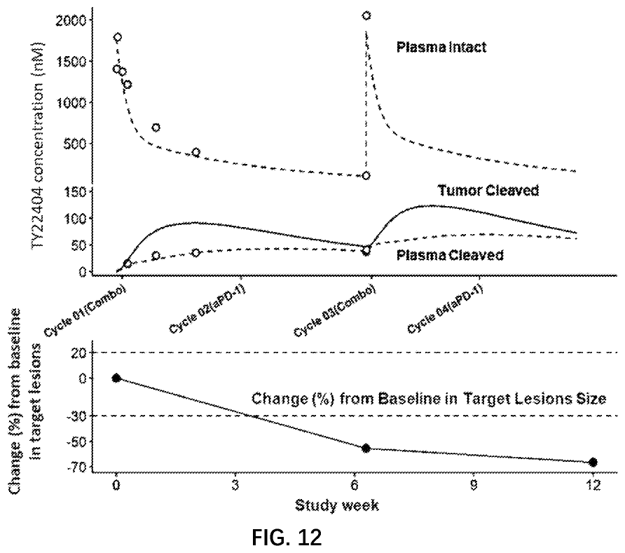

Case Study #3: A 58 year-old patient with advanced rectal adenocarcinoma patient with brain, lung and LN metastasis who received TY22404 10 mg/kg Q6W+pembrolizumab 200 mg Q3W. The PD-L1 CPS score of this patient is 0, the TMB is 7 Muts/Mb from ctDNA. This patient previously received 2 lines of therapies: FOLFOXIRI+Bev; 5-FU+XRT. As shown in Table 9, confirmed PD due to new lesion with 67% reduction in target lesions at the end of C4 (initial PR with 56% reduction at the end of cycle 2) was observed. Greater fluctuations in mPBPK model-predicted tumor cleaved PK, as well as reduced cleaved AUC/Cmax compared with 10 mg/kg Q3W dosing (see FIG. 12).

TABLE 9
Tumor Assessments in a Patient with Advanced Rectal Adenocarcinoma

| | Lesion # | Location | Baseline | End of C2 | End of C4 |
|---|---|---|---|---|---|
| Target lesion | TL#1 | Paratracheal LN | 16 mm | 7 mm | 6 mm |
| | TL#2 | Subcarinal LN | 20 mm | 9 mm | 6 mm |
| | Sum | | 36 mm | 16 mm | 12 mm |
| | | | | (−56%) | (−67%) |
| Non target lesions | | | Present | Present | Present |
| New lesion | | | | No | Yes |
| Overall response | | | | PR (−56%) | PD (mixed response) |

Case Study #4: A 66 year-old patient with advanced colorectal adenocarcinoma with lung metastasis who received TY22404 10 mg/kg Q3W+pembrolizumab 200 mg Q3W. MSS status, and the TMB is 11 Muts/Mb from ctDNA. This patient previously received 3 lines of therapies: Adjuvant FOLFOX; FOLFIRI+Vectibix; Rivoceranib (VEGF-R2)+TAS-102. As shown in Table 10, confirmed PD due to new lesion with 67% reduction in target lesions at the end of C4 (initial PR with 56% reduction at the end of cycle 2) was observed.

TABLE 10
Tumor Assessment in a Patient with Advanced Colorectal Adenocarcinoma

| | Lesion # | Location | Baseline | End of C2 | End of C4 |
|---|---|---|---|---|---|
| Target lesion | TL#1 | Right lung | 14 mm | 10 mm | 8 mm |
| | TL#2 | Right lung | 8 mm | 8 mm | 6 mm |
| | TL#3 | LN | 22 mm | 14 mm | 13 mm |
| | TL#4 | LN | 15 mm | 10 mm | 8 mm |
| | Total | | 59 mm | 42 mm | 35 mm |
| | | | | (−28.8%) | (−40.7%) |
| Non target | | | Present | Present | Present |
| lesions | | | | | |
| New lesion | | | | Yes (3 liver | All reduced |
| | | | | lesions) | |
| Overall | | | | iuPD | iPR |
| response | | | | | |

Case Study #5: A 66 year-old patient with HNSCC IO naïve (stage IVA) with lung metastasis who received TY22404 10 mg/kg Q6W+pembrolizumab 200 mg Q3W. The PD-L1 CPS score of this patient is 5. This patient previously received adjuvant therapy cisplatin, and 1 line of palliative therapies, docetaxel/cisplatin. As shown in Table 11, the patient showed partial response with 100% reduction in target lesions at the end of C4 and confirmed at C7.

TABLE 11
Tumor Assessment in a Patient with HNSCC

| | Lesion # | Location | Baseline | End of C2 | End of C4 |
|---|---|---|---|---|---|
| Target lesion | TL#1 | Paratracheal LN | 16 mm | 7 mm | 6 mm |
| | TL#2 | Subcarinal LN | 20 mm | 9 mm | 6 mm |
| | Sum | | 36 mm | 16 mm | 12 mm |
| | | | | (−56%) | (−67%) |
| Non target lesions | | | Present | Present | Present |
| New lesion | | | | No | Yes |
| Overall response | | | | PR (−56%) | PD (mixed response) |

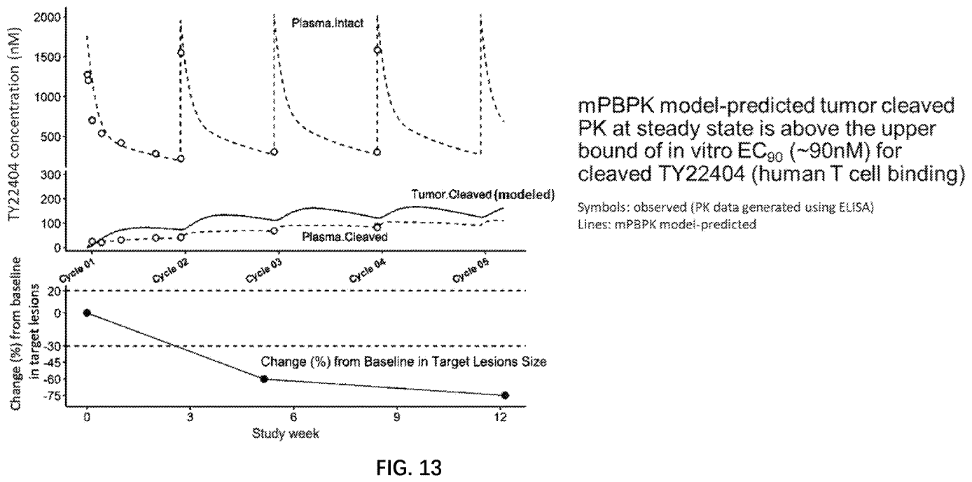

Case Study #6: A 55 year old patient with advanced colorectal adenocarcinoma, MSS, with mediastinal, paraaortic lymph nodes metastasis (stage IV) who received TY22404 10 mg/kg Q3W+pembrolizumab 200 mg Q3W. This patient previously received 2 lines of palliative therapies: FOLFOX+bevacizumab; FOLFIRI+aflibercept. As shown in Table 12, the patient showed PR with target lesions (lymph node) decreasing from 20 mm to 8 mm (in normal size of lymph node) at the end of C2 and the target lesion continue decreasing to 5 mm at the end of C4. TY22404 10 mg/kg Q3W+pembrolizumab 200 mg Q3W showed confirmed PR in MSS CRC, which also indicate model-informed PK and efficacy case studies support TY22404 dose selection of 10 mg/kg Q3W in microsatellite stable (MSS)-colorectal cancer (CRC) (FIG. 13).

TABLE 12
Tumor Assessment in a Patient with Advanced ColorectalTAdenocarcinoma

| | Lesion # | Location | Baseline | End of C2 | End of C4 |
|---|---|---|---|---|---|
| Target | TL#1 | Para-aortic LN | 20 mm | 8 mm | 5 mm |
| lesion | Target | | | CR | CR |
| | response | | | | |
| Non target | | | Present | Present | Present |
| lesions | | | | | |
| New lesion | | | | No | No |
| Overall | | | | PR (−60%) | PR (−75%) |
| response | | | | | |

### Biomarker Modulation in the Periphery

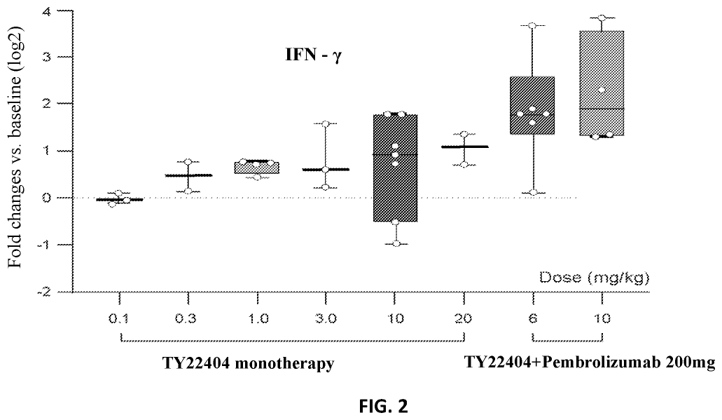

In TY22404 Phase I clinical trials, patients were enrolled in dose escalation cohorts from 0.1 mg/kg, 0.3 mg/kg, 1.0 mg/kg, 3.0 mg/kg, 10 mg/kg, and 20 mg/kg in monotherapy, as well as in combination with 200 mg pembrolizumab at 6 and 10 mg/kg of TY22404. Serum samples were prepared from peripheral blood collected at a series of visit time points (Cycle 1 Day 1 pre-dose, Cycle 1 Day 8, Cycle 1 Day 15, Cycle 2 Day 1 pre-dose, Cycle 3 Day 1 pre-dose, and Cycle 4 Day 1 pre-dose) according to standard protocol. The serum concentrations of a panel of proinflammatory cytokines including IFN-γ, TNF-α, IL-2, IL-6, etc., which are early responders of immune activation, were quantified by Mesoscale Discovery (MSD) Technologies' V-Plex Proinflammatory Panel 1 assay (Cat. No. K151A9H) following the manufacturer's instructions. As shown in FIG. 2, the fold changes of the peak IFN-γ levels from different patients relative to their corresponding baseline levels (Cycle 1 Day 1 pre-dose). Each dot represents the IFN-γ change from one patient. The results indicate that TY22404 monotherapy induces low levels of peripheral immune activation, as manifested by dose-dependent but limited increases of IFN-γ. But the magnitudes of IFN-γ increases, or immune activation, are more pronounced when TY22404 is combined with pembrolizumab.

### Example 2: Optimal Dose Selection of TY22404 with Significantly Widened Therapeutic Index Compared to Ipilimumab in Combination with Anti-PD-1 Antibodies Informed by QSP Modeling

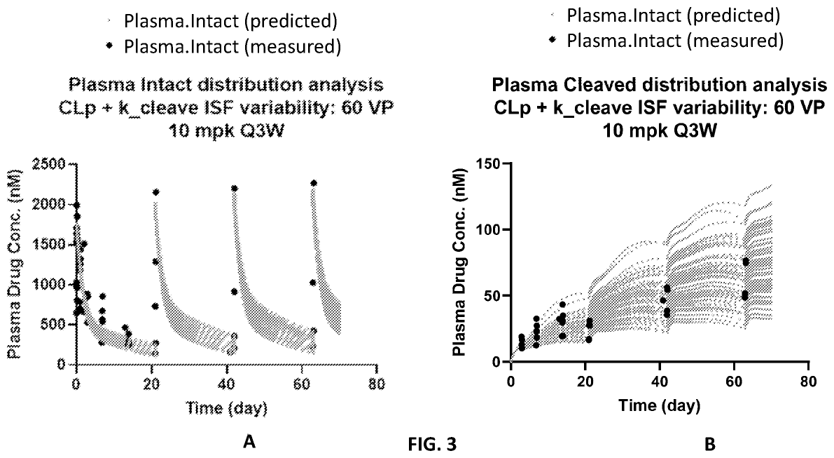

The developed mPBPK model can fit the observed pharmacokinetic (PK) data well across dose levels 10 mg/kg once every three weeks (Q3W). As shown in FIG. 3A and FIG. 3B, PK was used as a representative dose group, showing predicted (e.g., dashed lines) vs. measured (i.e. observed) plasma concentration for intact antibody and cleaved antibody, respectively. Despite the accumulation of cleaved TY22404 in plasma (FIG. 3B) over each dosing cycle, the simulated maximum steady-state cleaved TY22404 exposure (C_{max,ss}) at 10 mg/kg Q3W or Q6W (data not shown) dosing is ~6 or ~12 fold less than the mean C_{max,ss} of its parental Ab TY22404 (FIG. 3B) dosed at 3 mg/kg Q3W, respectively. These results are consistent with reduced circulating PD biomarkers, reflective of reduced whole-body immune activation and superior clinical safety profiles for TY22404. Hence, TY22404 can be administered safely as monotherapy or combination with other therapies (e.g., pembrolizumab).

### Example 3: Physiologically-Based Pharmacokinetic (mPBPK) Modeling for TY22404 Across Species

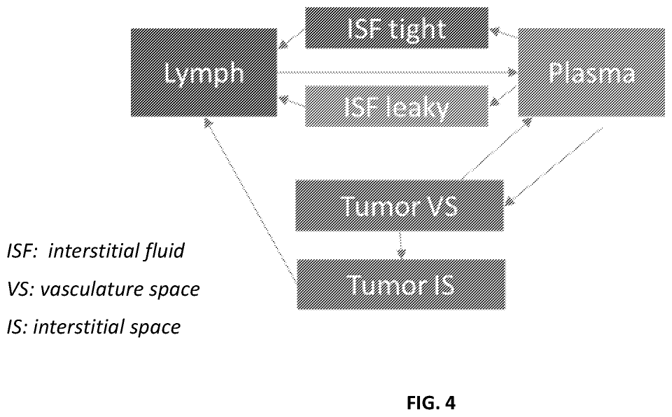

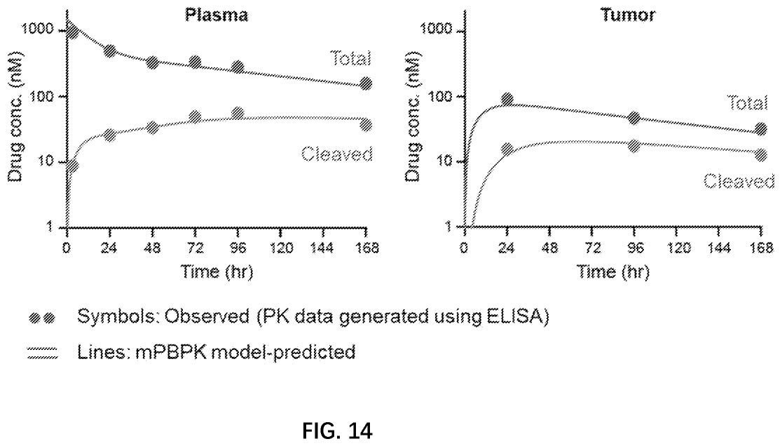

A minimal physiologically-based pharmacokinetic (mPBPK) model was developed to model total, intact, and cleaved forms of TY22404 following administration to different species (mice, rays, cynos and humans). Known molecular transformation and mass balance for total, intact and cleaved forms of TY22404 was integrated for all compartments. The antibody primarily circulates from the plasma compartment, through the ISF_leaky (i.e., interstitial fluid of leaky normal tissue) and ISF tight (i.e., interstitial fluid of tight normal tissue) compartments, into the lymph compartment and then back into plasma (see FIG. 4). Additional exchange occurs between the plasma compartment and the tumor_VS (i.e. tumor vasculature space) compartment, with a fraction continuing to circulate from the tumor_VS into the tumor_IS (i.e. tumor interstitial space) before flowing into the lymph. Clearance occurs from the plasma compartment only. mPBPK model can characterize plasma and tumor PK in tumor-bearing mice well after a 10 mg/kg single dose, allowing the estimation of tumor cleavage parameter of TY22404 (see FIG. 14). For tumor related parameters, measured PK data (e.g., tumor PK and plasma PK) in tumor-bearing mice was further modeled to estimate the tumor cleavage rate constant in mice and was kept the same for human modeling.

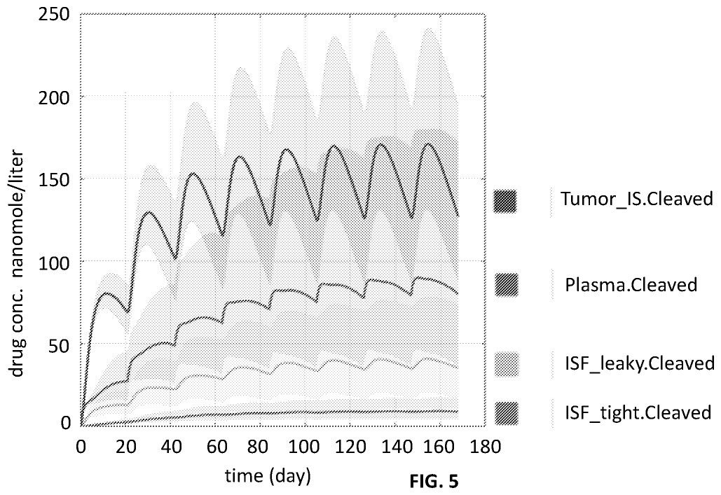

Using PK parameters from population model fitting to the observed PK data, simulations were conducted for virtual patients (VPs) to generate the mean PK and the variability (e.g., 95% Confidence Interval) at 10 mg/kg Q3W dosing. Simulated PK for the cleaved drug in tumor ISF is higher than the upper bound of EC₉₀s for human T cell binding for MMP9-cleaved TY22404 in vitro (see FIG. 5), supporting 10 mg/kg Q3W being an efficacious dose and is verified with emerging clinical efficacy data. Simulated PK for the cleaved drug in normal tissue interstitial fluid (ISF), including leaky and tight tissue, supported the superior safety profiles observed in the clinical for TY22404.

### Example 4: Predicted Tumor PK Comparison for Efficacy of TY22404 vs. Ipilimumab

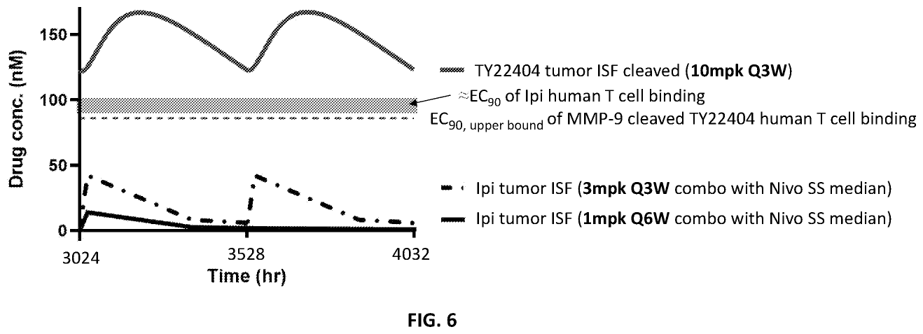

Ipilimumab (Ipi) predicted tumor ISF concentration at 1 mg/kg Q6W or 3 mg/kg Q3W intravenously cannot cover its EC_{90s, human T cell binding} during each specific dosing interval (see FIG. 6). Even at 3 mg/kg Q3W, the predicted tumor Cmax using 10% tumor tissue partition compared with plasma (i.e. systemic conc.) is about half of the in vitro EC₉₀s, human_{T cell binding}

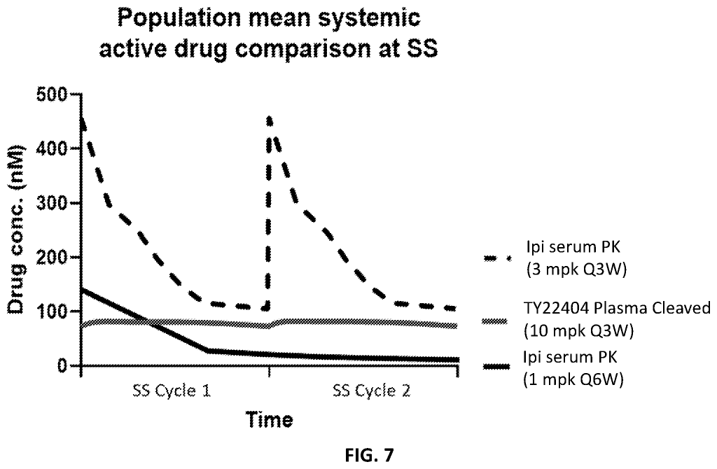

In contrast, as shown in FIG. 6, at steady-state (SS), maximum cleaved TY22404 tumor interstitial fluid (ISF) concentration at 10 mg/kg Q3W dosing is predicted on average to be significantly higher than ipilimumab for 3 mg/kg Q3W*4 doses or 1 mg/kg Q6W, respectively in the tumor microenvironment (TME). Simulated PK for the cleaved TY22404 in tumor ISF is higher than the upper bound of EC90s for human T cell binding for MMP9-cleaved TY22404 in vitro (e.g. dashed line). TY22404 is predicted to achieve higher target occupancy (RO >90%) throughout steady-state (SS) dosing cycle in TME at 10 mg/kg Q3W compared with ipilimumab at 3 mg/kg Q3W or 1 mg/kg Q6W. TY22404 (10 mg/kg Q3W) is also predicted to have reduced active drug exposures compared with ipilimumab (3 mg/kg Q3W) in normal tissue reflected by reduced systemic active drug PK (see FIG. 7). Taken together, PK modeling informed on enhanced therapeutic index (TI) of TY22404 over Ipilimumab in combination with anti-PD-1.

### Example 5: PK Modeling of Various TY22404 Dosing Schedules

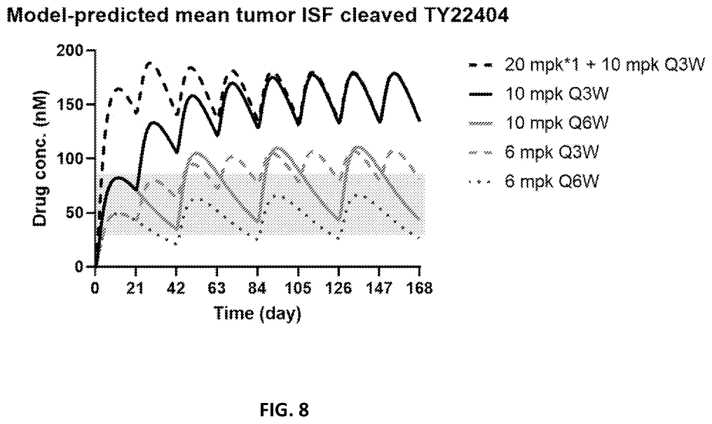

Interim TY22404 clinical PK data-based model prediction shows that the 10 mg/kg Q3W dosing regimen can likely cover the higher end of in vitro EC₉₀ₛ of cleaved TY22404 in the TME (see FIG. 8). Furthermore, as shown in FIG. 8, a 20 mg/kg loading dose+10 mg/kg Q3W dosing schedule can achieve tumor cleaved drug concentrations in cycle 1 similar to that with 10 mg/kg Q3W dosing at steady-state, potentially allowing for increased efficacy while maintaining safety as 10 mg/kg Q3W.

### Example 6: a Single Loading Dose Followed by Maintenance Doses Achieves Rapid Steady-State Plasma Concentrations of Cleaved TY22404

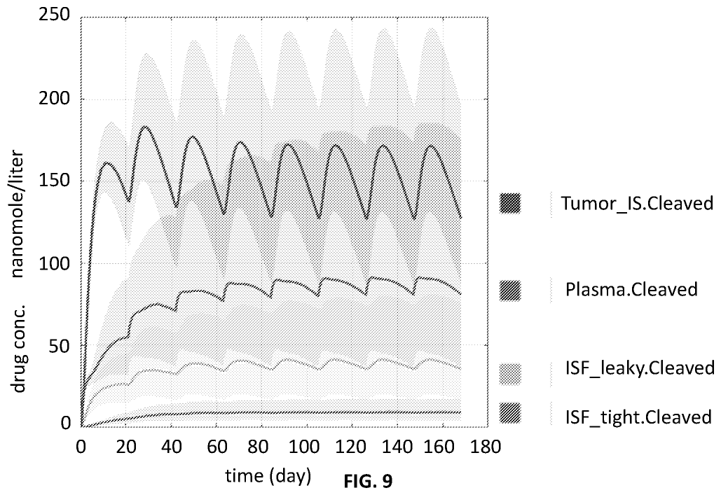

As shown in FIG. 9, the developed mPBPK model virtual patient simulations was applied to further investigate the role of a single loading dose followed by maintenance doses. The simulations suggest that a 20 mg/kg loading dose and 10 mg/kg Q3W maintenance dose is predicted to result in target steady-state plasma concentration in tumor ISF in cycle 1 (refer to Tumor_IS.Cleaved in FIG. 9). Despite the accumulation of cleaved TY22404 in plasma, the simulated maximum steady-state cleaved TY22404 exposure (C_{max,ss} refer to Plasma.Cleaved below Tumor_IS.Cleaved) at 20 mg/kg loading dose+10 mg/kg Q3W dosing is ~6 fold less than the mean C_{max,ss} of its parental antibody TY22404 dosed at 3 mg/kg Q3W, with manageable safety when combined with anti-PD-1 mAbs (e.g. pembrolizumab). Even when the 95% upper bound was considered for the population PK variability, it is still ~3 fold less than the mean C_{max,ss} of its parental antibody TY22404 dosed at 3 mg/kg Q3W, further supporting that this regimen should result in manageable safety as 10 mg/kg Q3W dosing, while potentially increasing efficacy in some patients.

### Example 7: Mechanism-Based Safety Modeling for TY22404 in Combination with Anti-PD1 Antibodies

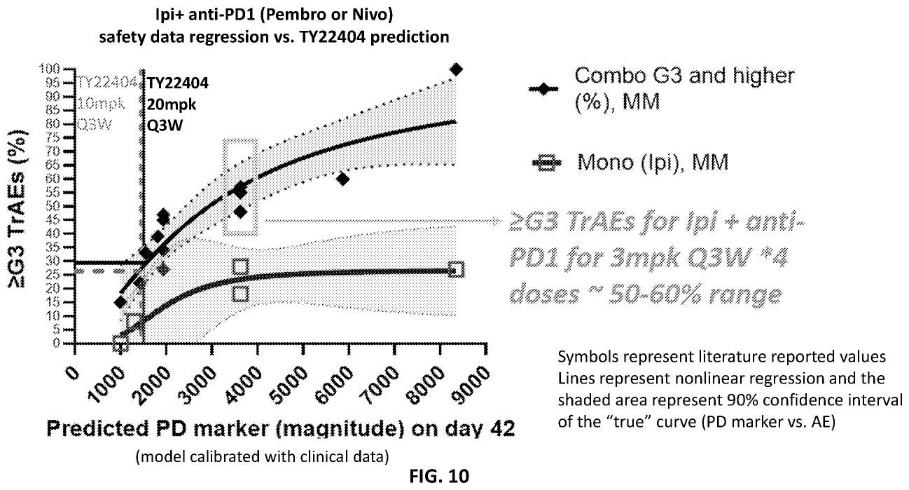

A novel mechanism-based model integrating pharmacokinetics (PK), pharmacodynamics (PD) and safety was constructed using published ipilimumab (Ipi), pembrolizumab (Pembro), Ipi+Pembro, Ipi+ nivolumab (Nivo) and tremelimumab (Treme) clinical and ex vivo data. As shown in FIG. 10, the model predicts that 10 mg/kg Q3W and 20 mg/kg Q3W of TY22404 shows clear advantage in treatment related adverse effects (TrAE) in comparison with Ipi 3 mg·kg Q3W*4 doses in a combination setting. Additionally, 20 mg/kg Q3W repeat dosing resulted in slightly increased ≥G3 TrAEs (e.g., <10% increase in absolute mean value) compared with 10 mg/kg Q3W repeat dosing of TY22404. Emerging clinical safety data for TY22404 is consistent with the predicted TrAE ranges (e.g., 10 mg/kg Q3W repeat dosing resulted in <20%≥G3 TrAEs from initial safety readout).

### Example 8: Dose Escalation Phase (Clinical Trial Design)-TY22404 in Combination with Pembrolizumab

A mTPI design with a target dose limiting toxicity (DLT) rate of approximately 20% and the equivalence interval (EI) of [0.15, 0.23], in which any dose is considered as a potential candidate for the true maximum tolerated dose (MTD), was applied for dose escalation and confirmation to determine RP2D for TY22404 in combination with pembrolizumab. Dose levels are shown in Table 13.

TABLE 13
TY22404-Pembrolizumab Dose Levels

| | Dose Level | TY22404 | Pembrolizumab |
|---|---|---|---|
| Design | (DL) | (mg/kg, Q3W and/or Q6W) | (mg, Q3W) |
| mTPI design | DL1 | ≤6 | 200 |
| | DL2 | ≤10 | 200 |
| | DL3 | 20 | 200 |

DL = dose level; mTPI = modified Toxicity Probability Interval; Q3W = every 3 weeks; Q6W = every 6 weeks.
DL1: Dosing regimen is to be further determined based on clinical data from initial repeat dosing at 6 mg/kg. Reduced dosing frequency (e.g., Q6W dosing) is an alternative schedule if Q3W dosing is not well tolerated. DL2 and DL3: These doses and dosing regimens are to be sequentially determined based on clinical data from DL1 and DL2, respectively. Loading dose(s) (e.g., ≤20 mg/kg) followed by maintenance dose at reduced dose levels (e.g., ≤10 mg/kg, including 3 or 6 mg/kg Q3W) and/or reduced dosing frequency (e.g., Q6W dosing) is allowed.

The dose escalation will start at 6 mg/kg Q3W following the mTPI design (DL1). Should this be tolerated based on early or late toxicity per SRC review, dosing will proceed to 10 mg/kg Q3W. If 6 mg/kg Q3W is not tolerated based on early or late toxicities per SRC review, then dosing will proceed at 6 mg/kg Q6W. If 6 mg/kg Q6W is tolerated, based on SRC review of early and late toxicities, then dosing may proceed at 10 mg/kg Q6W. Similarly, if 6 mg/kg Q3W is tolerated, but 10 mg/kg Q3W is not tolerated, based on SRC review of early and late toxicities, then dosing may proceed at 10 mg/kg Q6W. Furthermore, if 10 mg/kg Q3W is tolerated, based on SRC review of early and late toxicities, then dosing may proceed at 20 mg/kg Q3W. For the 20 mg/kg dose escalation cohort, only the patients with MSS CRC (<50% with liver metastasis) and 2L anti-PD-1/L1 experienced NSCLC are allowed. Based on the totality of the data, the SRC will designate the doses/schedules for the dose expansion phase.

DLTs were assessed using the National Cancer Institute Common Terminology Criteria for Adverse Events (NCI CTCAE) v5.0. The safety and tolerability of each TY22404 dose level was assessed by SRC after all patients enrolled in the dose level have been followed for at least 21 days after the first dose of TY22404-pembrolizumab combination regimen (DLT observation period). The dose and dosing frequency of pembrolizumab will not be changed.

Dose-dependent toxicity of anti-CTLA-4 therapies has severely limited their efficacy and therapeutic index (TI). Ipilimumab, the first FDA approved anti-CTLA-4 therapy for monotherapy and in combination with anti-PD-1 therapy, is limited due to safety concerns by dose level, frequency and cycles that may not maximize anti-tumor efficacy. The second FDA approved anti-CTLA-4 antibody, tremelimumab faces similar challenges in combination despite efficacy in front-line settings with limited number of doses. Next generation anti-CTLA-4 therapies must achieve better efficacy with an improved TI that allows for repeat dosing and sufficiently active dose levels. TY22404, a masked anti-CTLA-4 SAFEbody, is designed to allow for repeat dosing at active dose levels due to its improved TI by targeting a unique and highly conserved epitope of CTLA-4 on Treg cells in tumor microenvironment (TME) that is preferentially enriched and activated to enable CTLA-4-mediated depletion of Tregs in TME via epitope dependent effector functions such as ADCC, etc. Optimal dose selection of TY22404 in combination with anti-PD-1 antibodies requires quantitatively assessing different dosing regimens including PK/PD modeling of effects of plasma/intratumoral masked vs cleaved drug concentrations on efficacy and safety, etc. The species cross-reactivity of fully activated TY22404 or ADG116 enables quantitative approaches for TI assessment through seamless integration of preclinical and clinical data to predict cleaved TY22404 in TME in patients vs in vivo animal models using the same molecule, with a unified set of physiologically relevant parameters for population PK modeling for more than 50 patients across trials.

A quantitative systems pharmacology (QSP) model was developed by incorporating drug-specific dosage across melanoma trials into a published model (Kumar R, Thiagarajan K, Jagannathan L, et al. CPT Pharmacomet Syst Pharmacol. 2021; 10 (7): 684-695.) evaluating ipilimumab and pembrolizumab. Data from ipilimumab and nivolumab were further used. Characteristics of TY22404 were integrated by mPBPK modeling (Park J, Ariyapperuma M, Richardson G, et al. Journal of Clinical Oncology 2023, 41, no. 16_suppl). Furthermore, a novel safety model was developed incorporating data for ipilimumab, tremelimumab, pembrolizumab and nivolumab. In a hot tumor, 10 mg/kg Q3W TY22404 is predicted to result in comparable tumor objective response rate as ipilimumab 3 mg/kg Q3W*4 with anti-PD-1, but with significantly improved safety. In a colder and greater tumor burden scenario, 10 mg/kg or higher Q3W dosing of TY22404 led to better predicted efficacy than ipilimumab 3 mg/kg Q3W*4. The safety model further predicted >2-fold reduction in ≥G3 combination TRAEs by 10 mg/kg Q3W TY22404 vs. ipilimumab 3 mg/kg Q3W*4 (Jedd D. Wolchok, et al. N Engl J Med 2017. 377 (14): p. 1345-1356.), confirmed by TY22404 clinical findings.

Unique molecular design and properties of masked SAFEbody TY22404 allows for meaningful mPBPK and QSP modeling assessment of translational and clinical studies. These models predict an increased TI of TY22404 compared to ipi both as monotherapy or in combination with anti-PD-1. The widened TI of TY22404 enables TY22404 10 mg/kg Q3W repeat dosing with anti-PD-1, resulting in significantly increased CTLA-4 engagement by activated TY22404 in steady-state in tumors vs circulating blood. Initial clinical data support that TY22404 provides greater clinical benefit in combination with anti-PD-1, showing clinical responses in MSS CRC etc. driven by better target engagement in TME, while maintaining a favorable safety profile.

### Example 9: Loading Dose Selection of TY22404 with Predicted Increased Therapeutic Benefit

Exposure-response (E-R) analysis of available safety and efficacy data and the previously developed mPBPK model virtual patient simulations were used to further investigate the efficacy of additional loading dose regimens, including: (1) two 20 mg/kg Q3W loading doses followed by 10 mg/kg Q3W maintenance doses and (2) a single TY22404 loading dose between 30-50 mg/kg Q3W followed by 10 mg/kg Q3W maintenance doses.

A maintenance dose of 10 mg/kg Q3W TY22404 in combination with pembrolizumab was chosen for analysis because this regimen appears to improve clinical efficacy without worsening the safety profile as compared to pembrolizumab monotherapy or TY22404/pembrolizumab combination treatment with a maintenance dose of 6 mg/kg Q3W or 10 mg/kg Q6W Ty22404 over 14 cycles. Based on mPBKB modeling of TY22404 pharmacokinetic data, mg/kg Q3W TY22404 is expected to best cover target efficacious exposures at steady-state. Further, TY22404/pembrolizumab combination therapy had a similar rate of G3 treatment related adverse effects (TrAEs) as pembrolizumab monotherapy, with a 20% G3 TrAE rate at 6 mg/kg Q3W TY22404 and a 12.5% G3 TrAE rate at 10 mg/kg Q3W and no G4 or G5 TrAEs observed.

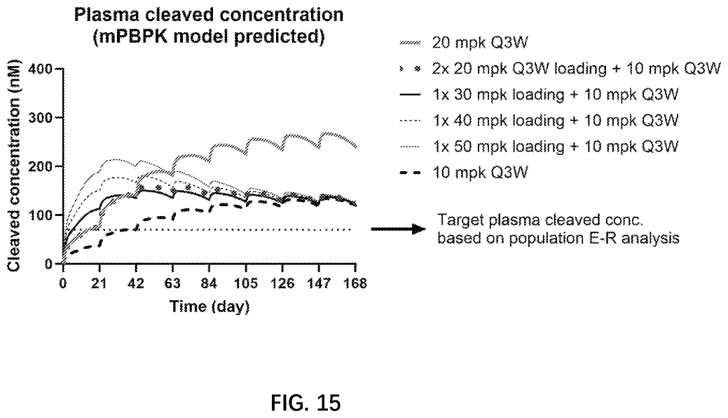

As shown in FIG. 15, virtual patient simulations show that a single loading dose of 30 mg/kg or higher have higher probabilities of achieving ~70 nM plasma cleaved TY22404, which is the target efficacious plasma concentration based on the current population E-R analysis, as compared to 10 mg/kg Q3W without an initial higher concentration loading dose, in cycle 1. In comparing the predicted cycle 1 plasma cleaved pharmacokinetics between a 20 mg/kg loading dose or 30 mg/kg loading dose, the anticipated plasma concentrations differ at 80 nM versus 120 nM, respectively. Therefore, clinical investigation is warranted to verify if achieving the target concentration early in cycle 1 using a higher loading dose, such as 30 mg/kg or higher, increases overall clinical response. Taken together, this data indicates that the additional loading dose regimens could increase therapeutic benefit.

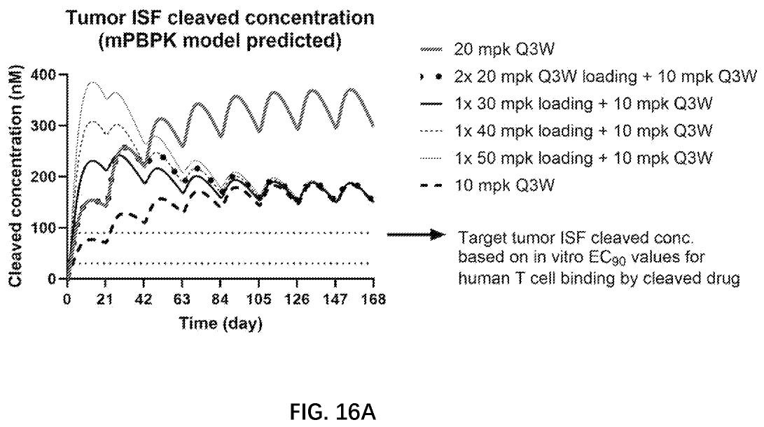

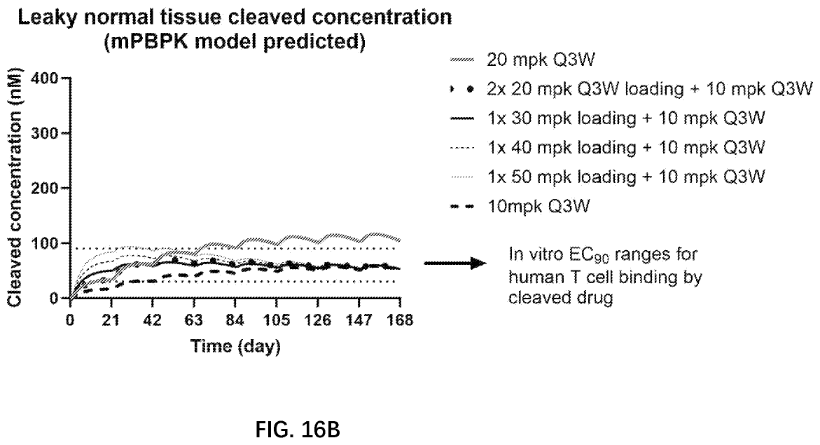

Further, FIG. 15 also shows that the model-predicted plasma concentrations of cleaved drug in cycles 2-4 are similar between the use of a 30 mg/kg or higher single loading dose and the use of two 20 mg/kg Q3W loading doses. This finding supports using safety information gained from two loading doses at 20 mg/kg Q3W to allow for the introduction of a 30 mg/kg or higher single loading dose, followed by a 10 mg/kg Q3W maintenance dose. In addition, as shown in FIG. 16A, modeling estimated that the interstitial fluid (ISF) maximum cleaved TY222404 concentration at 30 mg/kg in cycle 1 was >2-fold of the in vitro EC90 upper bound (e.g., 90 nM) of the human T cell binding assay described in Example 3, which can account for translational and modeling uncertainty, such as tumor cleavage in patients and PK variabilities at a population level. Additionally, as shown in FIG. 16B, the model estimated leaky normal tissue interstitial fluid maximum cleaved TY22404 concentrations in cycle 1 and 2 using a 30 mg/kg single loading dose to be ~1.5-2 fold less than the in vitro EC90 upper bound (e.g., 90 nM). The simulation also predicted that there is no difference in steady-state exposure when comparing any of the proposed loading dose regimens. Taken together, this data indicates that the proposed single 30-50 mg/kg loading dose regimens may be well-tolerated and have acceptable safety profiles in combination with pembrolizumab.

In summary, a single loading dose of 30-50 mg/kg followed by 10 mg/kg Q3W maintenance doses is predicted to approach target plasma cleaved drug concentration that two loading doses of 20 mg/kg Q3W reaches in cycle 2 one cycle earlier, thus increasing clinical efficacy and streamlining dose escalation, yet also maintain the safety of the 10 mg/kg Q3W maintenance dose based on a steady-state level of cleaved TY22404 that is below the predicted maximum leaky tissue exposure. Thus, the proposed loading dose regimens may increase clinical efficacy while maintaining safety profiles.

EXEMPLARY SEQUENCES
SEQ ID NO: 23 HVR-H1 Activatable anti-CTLA4
YSISSGYHWSWI
SEQ ID NO: 35 HVR-H2 Activatable anti-CTLA4
LARIDWDDDKYYSTSLKSRL
SEQ ID NO: 45 HVR-H3 Activatable anti-CTLA4
ARSYVYFDY
SEQ ID NO: 58 HVR-L1 Activatable anti-CTLA4
RASQSVRGRFLA
SEQ ID NO: 66 HVR-L2 Activatable anti-CTLA4
DASNRATGI
SEQ ID NO: 75 HVR-L3 Activatable anti-CTLA4
YCQQSSSWPPT
SEQ ID NO: 87
EVQLVESGGGLVQPGGSLRLSCAASGYSISSGYHWSWIRQAPGKG
LEWLARIDWDDDKYYSTSLKSRLTISRDNSKNTLYLQLNSLRAED
TAVYYCARSYVYFDYWGQGTLVTVSS
SEQ ID NO: 100:
RFLAWYQQKPGKAPKLLIYDASNRATGIPSRFSGSGSGTDFTLTI
SSLQPEDFATYYCQQSSSWPPTFGQGTKVEIKR
SEQ ID NO: 192 Masking Moiety (MM)
EVGSYPNPSSDCVPYYYACAY
SEQ ID NO: 221 Cleavable Moiety (CM)
SGRSAGGGGTPLGLAGSGGS
SEQ ID NO: 200 Masking Moiety (MM) plus
Cleavable Moiety (CM)
EVGSYPNPSSDCVPYYYACAYSGRSAGGGGTPLGLAGSGGS
SEQ ID NO: 320 Activatable anti-CTLA4 2 full
heavy chain (minus C-terminal lysine)
EVQLVESGGGLVQPGGSLRLSCAASGYSISSGYHWSWIRQAPGKG
LEWLARIDWDDDKYYSTSLKSRLTISRDNSKNTLYLQLNSLRAED
TAVYYCARSYVYFDYWGQGTLVTVSSASTKGPSVFPLAPSSKSTS
GGTAALGCLVKDYFPEPVTVSWNSGALTSGVHTFPAVLQSSGLYS
LSSVVTVPSSSLGTQTYICNVNHKPSNTKVDKKVEPKSCDKTHTC
PPCPAPELLGGPSVFLFPPKPKDTLMISRTPEVTCVVVDVSHEDP
EVKFNWYVDGVEVHNAKTKPREEQYNSTYRVVSVLTVLHQDWLNG
KEYKCKVSNKALPAPIEKTISKAKGQPREPQVYTLPPSRDELTKN
QVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 321 Activatable anti-CTLA4 2 full
heavy chain (with C-terminal lysine)
EVQLVESGGGLVQPGGSLRLSCAASGYSISSGYHWSWIRQAPGKG
LEWLARIDWDDDKYYSTSLKSRLTISRDNSKNTLYLQLNSLRAED
TAVYYCARSYVYFDYWGQGTLVTVSSASTKGPSVFPLAPSSKSTS
GGTAALGCLVKDYFPEPVTVSWNSGALTSGVHTFPAVLQSSGLYS
LSSVVTVPSSSLGTQTYICNVNHKPSNTKVDKKVEPKSCDKTHTC
PPCPAPELLGGPSVFLFPPKPKDTLMISRTPEVTCVVVDVSHEDP
EVKENWYVDGVEVHNAKTKPREEQYNSTYRVVSVLTVLHQDWLNG
KEYKCKVSNKALPAPIEKTISKAKGQPREPQVYTLPPSRDELTKN
QVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 322 Activatable anti-CTLA4 2 full
light chain with N-terminal masking moiety
and linker
EVGSYPNPSSDCVPYYYACAYSGRSAGGGGTPLGLAGSGGSDIQL
TQSPSSLSASVGDRVTITCRASQSVRGRFLAWYQQKPGKAPKLLI
YDASNRATGIPSRFSGSGSGTDFTLTISSLQPEDFATYYCQQSSS
WPPTFGQGTKVEIKRTVAAPSVFIFPPSDEQLKSGTASVVCLLNN
FYPREAKVQWKVDNALQSGNSQESVTEQDSKDSTYSLSSTLTLSK
ADYEKHKVYACEVTHQGLSSPVTKSENRGEC
SEQ ID NO: 323 Activatable anti-CTLA4 1 full
heavy chain (minus C-terminal lysine)
EVQLVESGGGLVQPGGSLRLSCAASGYSISSGYHWSWIRQAPGKG
LEWLARIDWDDDKYYSTSLKSRLTISRDNSKNTLYLQLNSLRAED
TAVYYCARSYVYFDYWGQGTLVTVSSASTKGPSVFPLAPSSKSTS
GGTAALGCLVKDYFPEPVTVSWNSGALTSGVHTFPAVLQSSGLYS
LSSVVTVPSSSLGTQTYICNVNHKPSNTKVDKKVEPKSCDKTHTC
PPCPAPELLGGPSVFLFPPKPKDTLMISRTPEVTCVVVDVSHEDP
EVKENWYVDGVEVHNAKTKPREEQYNSTYRVVSVLIVLHQDWLNG
KEYKCKVSNKALPAPIEKTISKAKGQPREPQVYTLPPSRDELTKN
QVSLTCLVKGFYPSDIAVEWESNGQPENNYKTTPPVLDSDGSFFL
YSKLTVDKSRWQQGNVFSCSVMHEALHNHYTQKSLSLSPG

What is claimed is:

1. A method of treating a cancer in a subject, comprising administering to the subject:
(a) an effective amount of an activatable antibody, wherein the activatable antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75), and wherein the activatable antibody further comprises: a polypeptide covalently attached to the N-terminus of the light chain of the anti-CTLA4 antibody, said polypeptide comprising, from N-terminus to C-terminus, a masking moiety (MM) and a cleavable moiety (CM), wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221); and
(b) an effective amount of pembrolizumab,
wherein the activatable antibody is administered at a dose of from about 6 mg/kg to about 30 mg/kg once every 3 to 6 weeks, and wherein the pembrolizumab is administered at a dose of from about 100 mg to about 300 mg once every three weeks or about 200 mg to about 600 mg every six weeks.

2. The method of claim 1,
wherein the activatable antibody is administered at a dose of from about 6 mg/kg to about 10 mg/kg once every 3 to 6 weeks.

3. The method of claim 1, wherein the activatable antibody is administered at a dose of from about 10 mg/kg to about 20 mg/kg once every 3 to 6 weeks.

4. The method of claim 1, wherein the activatable antibody is administered at a dose of from about 20 mg/kg to about 30 mg/kg once every 3 to 6 weeks.

5. The method of claim 1, wherein the activatable antibody is administered at a dose of 10 mg/kg once every 3 weeks.

6. The method of claim 1, wherein the activatable antibody is administered at a dose of 20 mg/kg once every 3 weeks.

7. The method of claim 1, wherein the activatable antibody is administered at a dose of 20 mg/kg once every 6 weeks.

8. The method of claim 1, wherein the activatable antibody is administered at a dose of 30 mg/kg once every 6 weeks.

9. The method of claim 1, wherein the activatable anti-CTLA4 antibody is administered at a dose of about 6 mg/kg once every 3 to 6 weeks.

10. The method of any one of claim 1-6, wherein the pembrolizumab is administered at a dose of about 200 mg once every 3 weeks.

11. The method of claim 1, 7, or 8, wherein the pembrolizumab is administered at a dose of about 400 mg once every six weeks.

12. The method of any one of claims 1-11, wherein the cancer is resistant or refractory to a prior therapy, wherein the prior therapy is an inhibitor of CTLA4, PD-1, or a PD-1 ligand.

13. The method of claim 12, wherein the prior therapy is ipilimumab.

14. The method of any one of claims 1-13, wherein the cancer is colorectal cancer.

15. The method of claim 14, wherein the CRC is microsatellite stable (MSS) CRC.

16. The method of claim 15, wherein the MSS CRC has not metastasized to the liver.

17. The method of claim 15, wherein the MSS CRC has not metastasized to the peritoneum.

18. The method of claim 15, wherein the MSS CRC has not metastasized to the liver or to the peritoneum.

19. The method of any one of claims 1-13, wherein the cancer is endometrium cancer.

20. The method of any one of claims 1-13, wherein the cancer is neuroendocrine cancer, cecum adenocarcinoma, pancreatic cancer, or ovarian cancer.

21. The method of any one of claim 1-15, 19, or 20 wherein the cancer is an advanced stage metastatic cancer.

22. The method of claim 21, wherein the cancer has metastasized to the lung or the liver.

23. The method of any one of claims 1-22, wherein the activatable anti-CTLA4 antibody comprises a heavy chain variable region comprising the amino acid sequence of SEQ ID NO: 87 or a variant thereof having at least about 90% sequence identity to the amino acid sequence of SEQ ID NO: 87, and a light chain variable region comprising the amino acid sequence of SEQ ID NO: 100 or a variant thereof having at least about 90% sequence identity to the amino acid sequence of SEQ ID NO: 100.

24. The method of claim 23, wherein the heavy chain variable region comprises the amino acid sequence of SEQ ID NO: 87 and the light chain variable region comprises the amino acid sequence of SEQ ID NO: 100.

25. The method of claim 24, wherein the activatable anti-CTLA4 antibody comprises a full heavy chain region of SEQ ID NO:320 or SEQ ID NO:321.

26. The method of claim 25, wherein the activatable anti-CTLA4 antibody comprises a full light chain region of SEQ ID NO:322 or SEQ ID NO:323.

27. The method of any one of claims 1-26, wherein the subject is human.

28. The method of any one of claims 1-27, wherein the activatable anti-CTLA4 antibody and the pembrolizumab are both administered on day 1 of the 3 to 6 week dosing schedule.

29. A method of treating a cancer in a subject, comprising administering to the subject an effective amount of an activatable antibody in combination with pembrolizumab, wherein the activatable antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75), and wherein the activatable antibody further comprises: a polypeptide covalently attached to the N-terminus of the light chain of the anti-CTLA4 antibody, said polypeptide comprising, from N-terminus to C-terminus, a masking moiety (MM) and a cleavable moiety (CM), wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221), wherein the activatable antibody is administered as one to three loading doses of from about 20 mg/kg to about 50 mg/kg followed by maintenance doses of from about 5 mg/kg to about 20 mg/kg once every three weeks or once every six weeks.

30. The method of claim 29, wherein the activatable antibody is administered as one to three loading doses of from about 20 mg/kg to about 40 mg/kg followed by maintenance doses of from about 6 mg/kg to about 20 mg/kg once every three weeks or once every six weeks.

31. The method of claim 29, wherein the activatable antibody is administered as one to three loading doses of from about 20 mg/kg to about 40 mg/kg followed by maintenance doses of from about 6 mg/kg to about 10 mg/kg once every three weeks or once every six weeks.

32. The method of claim 29, wherein the activatable antibody is administered as one to three loading doses of from about 20 mg/kg to about 40 mg/kg followed by maintenance doses of from about 10 mg/kg to about 20 mg/kg once every three weeks or once every six weeks.

33. The method of claim 29, wherein the activatable antibody is administered as one loading dose of from about 20 mg/kg to about 40 mg/kg followed by maintenance doses of from about 6 mg/kg to about 10 mg/kg once every three weeks or once every six weeks.

34. The method of any one of claim 29-33, wherein the loading doses are 20 mg/kg.

35. The method of any one of claim 29-33, wherein the loading doses are 30 mg/kg.

36. The method of any one of claim 29-33, wherein the loading doses are 40 mg/kg.

37. The method of any one of claims 29-36, wherein a single loading dose is administered to the subject prior to administration of the maintenance doses.

38. The method of any one of claims 29-37, wherein two loading doses are administered to the subject prior to administration of the maintenance doses.

39. The method of any one of claims 29-37, wherein three loading doses are administered to the subject prior to administration of the maintenance doses.

40. The method of any one of claims 29-39, wherein the maintenance doses are administered at 10 mg/kg.

41. The method of claim 40, wherein the maintenance doses are administered once every three weeks.

42. The method of claim 40, wherein the maintenance doses are administered once every six weeks.

43. The method of any one of claims 29-39, wherein the maintenance doses are administered at 20 mg/kg.

44. The method of claim 43, wherein the maintenance doses are administered once every three weeks.

45. The method of claim 43, wherein the maintenance doses are administered once every six weeks.

46. The method of any one of claims 29-45, wherein the first maintenance dose is administered three weeks after administration of the last loading dose.

47. The method of any one of claims 29-46, wherein the pembrolizumab is administered at a dose from about 100 mg to about 300 mg once every three weeks or about 200 mg to about 600 mg once every six weeks.

48. The method of any one of claims 29-47, wherein the cancer is resistant or refractory to a prior therapy, wherein the prior therapy is an inhibitor of CTLA4, PD-1, or a PD-1 ligand.

49. The method of claim 48, wherein the prior therapy is ipilimumab.

50. The method of any one of claims 29-47, wherein the cancer is colorectal cancer (CRC).

51. The method of claim 50, wherein the CRC is microsatellite stable (MSS) CRC.

52. The method of any one of claims 29-47, wherein the cancer is squamous cell carcinoma.

53. The method of any one of claims 29-47, wherein the cancer is anal squamous cell carcinoma or penile squamous cell carcinoma.

54. The method of any one of claims 29-47, wherein the cancer is pancreatic cancer.

55. The method of claim 54, wherein the cancer is pancreatic ductal adenocarcinoma (PDAC).

56. The method of any one of claims 29-47, wherein the cancer is ovarian cancer.

57. The method of any one of claims 29-47, wherein the cancer is NSCLC.

58. The method of any one of claims 29-47, wherein the cancer is hepatocellular carcinoma.

59. The method of any one of claims 29-58, wherein the cancer is an advanced stage metastatic cancer.

60. The method of claim 59, wherein the cancer has metastasized to the lung or the liver.

61. The method of any one of claims 29-60, wherein the activatable antibody comprises a heavy chain variable region comprising the amino acid sequence of SEQ ID NO: 87 or a variant thereof having at least about 90% sequence identity to the amino acid sequence of SEQ ID NO: 87, and a light chain variable region comprising the amino acid sequence of SEQ ID NO: 100 or a variant thereof having at least about 90% sequence identity to the amino acid sequence of SEQ ID NO: 100.

62. The method of claim 61, wherein the heavy chain variable region comprises the amino acid sequence of SEQ ID NO: 87 and the light chain variable region comprises the amino acid sequence of SEQ ID NO: 100.

63. The method of claim 62, wherein the activatable antibody comprises a full heavy chain region of SEQ ID NO:320 or SEQ ID NO:321.

64. The method of claim 62, wherein the activatable antibody comprises a full light chain region of SEQ ID NO:322 or SEQ ID NO:323.

65. The method of any one of claims 29-64, wherein the subject is human.

66. A method of treating a cancer in a subject, comprising administering to the subject an effective amount of an activatable antibody in combination with pembrolizumab, wherein the activatable antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75), and wherein the activatable antibody further comprises: a polypeptide covalently attached to the N-terminus of the light chain of the anti-CTLA4 antibody, said polypeptide comprising, from N-terminus to C-terminus, a masking moiety (MM) and a cleavable moiety (CM), wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221), wherein the activatable antibody is administered at a dose that provides a steady-state plasma concentration of the cleaved antibody of above the EC50 of the cleaved antibody.

67. A method of treating a cancer in a subject, comprising administering to the subject an effective amount of an activatable antibody in combination with pembrolizumab, wherein the activatable antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75), and wherein the activatable antibody further comprises: a polypeptide covalently attached to the N-terminus of the light chain of the anti-CTLA4 antibody, said polypeptide comprising, from N-terminus to C-terminus, a masking moiety (MM) and a cleavable moiety (CM), wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221), wherein the activatable antibody is administered at a dose that provides a steady-state plasma concentration of the cleaved antibody of above the EC90 of the cleaved antibody.

68. A method of treating a cancer in a subject, comprising administering to the subject an effective amount of an activatable antibody in combination with pembrolizumab, wherein the activatable antibody comprises an HVR-H1 comprising an amino acid sequence according to a formula YSISSGYHWSWI (SEQ ID NO: 23), an HVR-H2 comprising an amino acid sequence according to a formula LARIDWDDDKYYSTSLKSRL (SEQ ID NO: 35), an HVR-H3 comprising an amino acid sequence according to a formula ARSYVYFDY (SEQ ID NO: 45), an HVR-L1 comprising an amino acid sequence according to the formula RASQSVRGRFLA (SEQ ID NO: 58), an HVR-L2 comprises an amino acid sequence according to a formula DASNRATGI (SEQ ID NO: 66), and an HVR-L3 comprising an amino acid sequence according to a formula YCQQSSSWPPT (SEQ ID NO: 75), and wherein the activatable antibody further comprises: a polypeptide covalently attached to the N-terminus of the light chain of the anti-CTLA4 antibody, said polypeptide comprising, from N-terminus to C-terminus, a masking moiety (MM) and a cleavable moiety (CM), wherein the MM comprises an amino acid sequence EVGSYPNPSSDCVPYYYACAY (SEQ ID NO:192), and the cleavable moiety comprises an amino acid sequence SGRSAGGGGTPLGLAGSGGS (SEQ ID NO:221), wherein the activatable antibody is administered at a dose that provides a steady-state plasma concentration of the cleaved antibody of from about 100 nM to about 200 nM.

69. The method of claim 68, wherein the activatable antibody is administered at a dose that provides a steady-state plasma concentration of the cleaved antibody of from about 100 nM to about 175 nM.

70. The method of claim 68, wherein the activatable antibody is administered at a dose that provides a steady-state plasma concentration of the cleaved antibody of from about 100 nM to about 150 nM.

71. The method of claim 68, wherein the activatable antibody is administered at a dose that provides a steady-state plasma concentration of the cleaved antibody of from about 150 nM to about 200 nM.

72. The method of any one of claims 66-71, wherein the steady-state plasma concentration of the cleaved antibody is measured at the trough level of the anti-CTLA4 antibody.

73. The method of any one of claims 66-72, wherein the activatable antibody is administered as a single loading dose or two loading doses followed by maintenance doses, wherein the amount of the loading dose is higher than the amount of the maintenance doses.

74. The method of claim 73, wherein a single loading dose of the activatable antibody is administered to the subject prior to the administration of the maintenance doses.

75. The method of claim 74, wherein the loading dose is about 20 mg/kg.

76. The method of claim 74, wherein the loading dose is about 30 mg/kg.

77. The method of claim 74, wherein the loading dose is about 40 mg/kg.

78. The method of claim 74, wherein the loading dose is about 50 mg/kg.

79. The method of claim 73, wherein two loading dose of the activatable antibody are administered to the subject prior to the administration of the maintenance doses.

80. The method of claim 79, wherein the loading doses are about 20 mg/kg.

81. The method of claim 79, wherein the loading doses are about 30 mg/kg.

82. The method of claim 79, wherein the loading doses are about 40 mg/kg.

83. The method of claim 79, wherein the loading doses are about 50 mg/kg.

84. The method of any one of claims 73-83, wherein the maintenance doses are about 10 mg/kg.

85. The method of any one of claims 73-84, wherein the activatable antibody comprises a heavy chain variable region comprising the amino acid sequence of SEQ ID NO: 87 or a variant thereof having at least about 90% sequence identity to the amino acid sequence of SEQ ID NO: 87, and a light chain variable region comprising the amino acid sequence of SEQ ID NO: 100 or a variant thereof having at least about 90% sequence identity to the amino acid sequence of SEQ ID NO: 100.

86. The method of claim 85, wherein the heavy chain variable region comprises the amino acid sequence of SEQ ID NO: 87 and the light chain variable region comprises the amino acid sequence of SEQ ID NO: 100.

87. The method of claim 85, wherein the activatable antibody comprises a full heavy chain region of SEQ ID NO:320 or SEQ ID NO:321.

88. The method of claim 86 wherein the activatable antibody comprises a full light chain region of SEQ ID NO:322 or SEQ ID NO:323.

89. The method of any one of claims 73-88, wherein the subject is human.


SEQUENCE LISTING (323 sequences)

SEQ ID NO: 1-9 - skipped (9 entries)

SEQ ID NO: 10 - protein, 15 aa, synthetic construct
RASKGVSTSGYSYLH

SEQ ID NO: 11 - protein, 7 aa, synthetic construct
LASYLES

SEQ ID NO: 12 - protein, 9 aa, synthetic construct
QHSRDLPLT

SEQ ID NO: 13 - protein, 111 aa, synthetic construct [sequence omitted]

SEQ ID NO: 14 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 5 aa, synthetic construct
NYYMY

SEQ ID NO: 16 - protein, 17 aa, synthetic construct
GINPSNGGTNFNEKFKN

SEQ ID NO: 17 - protein, 11 aa, synthetic construct
RDYRFDMGFDY

SEQ ID NO: 18 - protein, 120 aa, synthetic construct [sequence omitted]

SEQ ID NO: 19 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 11 aa, synthetic construct
FTFSDYGIHWV

SEQ ID NO: 21 - protein, 12 aa, synthetic construct
YSISSGYHWDWI

SEQ ID NO: 22 - protein, 11 aa, synthetic construct
FTFSDYWIHWV

SEQ ID NO: 23 - protein, 12 aa, synthetic construct
YSISSGYHWSWI

SEQ ID NO: 24 - protein, 13 aa, synthetic construct
FSLSTGGVAVSWI

SEQ ID NO: 25 - protein, 13 aa, synthetic construct
FSLSTGGVAVGWI

SEQ ID NO: 26 - protein, 11 aa, synthetic construct
FTFSGYAIHWV

SEQ ID NO: 27 - protein, 11 aa, synthetic construct
YTFSGYGIHWV

SEQ ID NO: 28 - protein, 11 aa, synthetic construct
YTFSGYAIHWV

SEQ ID NO: 29 - protein, 12 aa, synthetic construct
YSITSGHYWSWI

SEQ ID NO: 30 - protein, 21 aa, synthetic construct
IGIISPSSGSTNYAQKFQGRV

SEQ ID NO: 31 - protein, 21 aa, synthetic construct
VSSISGSGSTTYYADSVKGRF

SEQ ID NO: 32 - protein, 20 aa, synthetic construct
IGEIYHSGSTYYSPSLKSRV

SEQ ID NO: 33 - protein, 21 aa, synthetic construct
VSGISGYGGSTYYADSVKGRF

SEQ ID NO: 34 - skipped

SEQ ID NO: 35 - protein, 20 aa, synthetic construct
LARIDWDDDKYYSTSLKSRL

SEQ ID NO: 36-44 - skipped (9 entries)

SEQ ID NO: 45 - protein, 9 aa, synthetic construct
ARSYVYFDY

SEQ ID NO: 46-57 - skipped (12 entries)

SEQ ID NO: 58 - protein, 12 aa, synthetic construct
RASQSVRGRFLA

SEQ ID NO: 59-65 - skipped (7 entries)

SEQ ID NO: 66 - protein, 9 aa, synthetic construct
DASNRATGI

SEQ ID NO: 67-74 - skipped (8 entries)

SEQ ID NO: 75 - protein, 11 aa, synthetic construct
YCQQSSSWPPT

SEQ ID NO: 76-86 - skipped (11 entries)

SEQ ID NO: 87 - protein, 116 aa, synthetic construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSS

SEQ ID NO: 88-99 - skipped (12 entries)

SEQ ID NO: 100 - protein, 109 aa, synthetic construct
DIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKR

SEQ ID NO: 101-147 - skipped (47 entries)

SEQ ID NO: 148 - protein, 5 aa, synthetic construct
EVGSY

SEQ ID NO: 149-191 - skipped (43 entries)

SEQ ID NO: 192 - protein, 21 aa, synthetic construct
EVGSYPNPSSDCVPYYYACAY

SEQ ID NO: 193-199 - skipped (7 entries)

SEQ ID NO: 200 - protein, 41 aa, synthetic construct
EVGSYPNPSSDCVPYYYACAYSGRSAGGGGTPLGLAGSGGS

SEQ ID NO: 201-220 - skipped (20 entries)

SEQ ID NO: 221 - protein, 20 aa, synthetic construct
SGRSAGGGGTPLGLAGSGGS

SEQ ID NO: 222-319 - skipped (98 entries)

SEQ ID NO: 320 - protein, 445 aa, synthetic construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 321 - protein, 446 aa, synthetic construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 256 aa, synthetic construct
EVGSYPNPSSDCVPYYYACAYSGRSAGGGGTPLGLAGSGGSDIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 323 - protein, 445 aa, synthetic construct; identical to SEQ ID NO: 320